(19) United States
(12) Patent Application Publication
(10) Pub. No.: US 2024/0175044 A1
(43) Pub. Date: May 30, 2024

(54) HIGH GROWTH AND HIGH HARDINESS TRANSGENIC PLANTS

(71) Applicant: Biolumic Limited, Palmerston North (NZ)

(72) Inventors: Jason John WARGENT, Palmerston North (NZ); Claudia Rossig, Palmerston North (NZ)

(21) Appl. No.: 18/336,270

(22) Filed: Jun. 16, 2023

Related U.S. Application Data
(63) Continuation of application No. 17/984,487, filed on Nov. 10, 2022, now abandoned.
Application No. 17/984,487 is a continuation of application No. 16/796,145, filed on Feb. 20, 2020, now abandoned.
Application No. 16/796,145 is a continuation of application No. PCT/IB2018/001056, filed on Aug. 21, 2018.
(60) Provisional application No. 62/548,271, filed on Aug. 21, 2017.

(51) Int. Cl. C12N 15/82

(52) U.S. Cl. CPC C12N 15/8273; C12N 15/8294; C12N 15/8295; C12N 15/8297; C12N 15/8298

(57) ABSTRACT

Aspects of the disclosure relate to systems and methods for enhancing plant performance by identifying and manipulating the expression of plant genes involved in UV-B mediated improvements to hardiness and growth. Some aspects of the disclosure relate to systems and methods for identifying plant novel genes responsive to light stimulation. Some aspects of the disclosure relate to systems and methods for identifying transgenic plants improved so as to present desired agronomic traits associated with UV-B light stimulation. Some aspects of the disclosure relate to systems and methods for modulating plant sensitivity to light for enhancing plant performance or a desired agronomic trait. Some aspects of the disclosure relate to systems and methods for generating stable transgenic plants that exhibit a desired agronomic trait.

### CROSS-REFERENCE

This application is a continuation of U.S. patent application Ser. No. 17/984,487, filed Nov. 10, 2022, which is a continuation of U.S. patent application Ser. No. 16/796,145, filed Feb. 20, 2022, now abandoned, which is a continuation of International Patent Application No. PCT/IB2018/001056, filed Aug. 21, 2018, which claims the benefit of priority to U.S. Provisional Patent Application No. 62/548,271 filed Aug. 21, 2017, which is incorporated herein by reference in its entirety.

### BACKGROUND

The United Nations projects the current world population of about 7.3 billion to reach 9.7 billion in 2050 and 11.2 billion in 2100 (UN World Population Prospects: The 2015 Revision). On average, nearly 353,000 babies are born and are added to the world food demand each day (The United Nations Children's Emergency Fund). There is an important societal and commercial impetus to find ways of improving yield and quality of crops for human consumption, and doing so in a safe and sustainable manner. Increased fertilizer application and more water usage through irrigation have increased crop yield for over 70% in the past. In some other approaches, pesticides are used to protect seeds and plants from pest and/or diseases. Yet, adding chemical agents, e.g., fertilizer, can sometimes be deleterious on another biochemical pathway, cause a negative phenotype, or cause environmental pollution. Further, pesticides can release toxicity to non-target insects, fungi or bacteria. A substantial share of the increasing food demand could be met by producing crops with higher yield or quality without the use of chemical agents. At the same time, this would support a growing green economy and greatly reduce pressures on biodiversity and water resources. Provided herein are systems and methods utilizing physical treatments on seeds or plants to improve plant performance and subsequent yield or quality of crops.

### SUMMARY

In general, the present disclosure relates to identifying modulators of light sensitivity in a plant for enhancing abiotic stress resistance, biotic stress resistance, growth, yield, and hardiness. The present disclosure also relates to generating stable transgenic plants with desired agronomic traits.

Transgenic plants with improved agronomic traits such as yield, environmental stress resistance, pest resistance, herbicide tolerance, improved seed compositions, and the like are desired by both farmers and consumers. Although considerable efforts in plant breeding have provided significant gains in desired traits, the ability to introduce specific DNA into plant genomes provides further opportunities for generation of plants with improved and/or unique traits. Transgenic plant with stable integrated DNA for a desired trait or an enhanced agronomic trait may be generated using systems and methods described herein.

In some instances, an aspect of the present disclosure provides a method for modulating photomorphogenesis.

Provided herein are methods for identifying a modulator of a UVR8-COP1-HY5 UV-B signaling pathway that improves growth in a plant, the method comprising: irradiating plant material using light having an enriched wavelength between 280-320 nm; selecting the plant material having at least one physiological condition selected from the group consisting of enhanced crop yield, growth rate, hardiness, stress resistance, root growth, root architecture, and pathological resistance compared to a plant material lacking the irradiation; and identifying a gene that is associated with the at least one physiological condition. Further provided herein are methods for identifying a modulator of a UVR8-COP1-HY5 UV-B signaling pathway that improves growth in a plant, wherein the plant material is exposed to an enriched wavelength of about 286 nm. Further provided herein are methods for identifying a modulator of a UVR8-COP1-HY5 UV-B signaling pathway that improves growth in a plant, wherein the plant material is exposed to an enriched wavelength of 286 nm prior to a subsequent growth phase of a seedling. Further provided herein are methods for identifying a modulator of a UVR8-COP1-HY5 UV-B signaling pathway that improves growth in a plant, wherein the plant material is exposed to an enriched wavelength of about 280 nm. Further provided herein are methods for identifying a modulator of a UVR8-COP1-HY5 UV-B signaling pathway that improves growth in a plant, wherein the plant material is exposed to an enriched wavelength of 280 nm prior to a subsequent growth phase of a seedling. Further provided herein are methods for identifying a modulator of a UVR8-COP1-HY5 UV-B signaling pathway that improves growth in a plant, further comprising determining a nucleic acid sequence of the gene that is associated with the at least one physiological condition. Further provided herein are methods for identifying a modulator of a UVR8-COP1-HY5 UV-B signaling pathway that improves growth in a plant, wherein the determining comprises at least one of nucleic acid sequencing, microarray, quantitative-polymerase chain reaction, Western blot, and immunohistochemistry analysis. Further provided herein are methods for identifying a modulator of a UVR8-COP1-HY5 UV-B signaling pathway that improves growth in a plant, wherein the root architecture comprises at least one of nodule formation, root growth, and spatial configuration. Further provided herein are methods for identifying a modulator of a UVR8-COP1-HY5 UV-B signaling pathway that improves growth in a plant, further comprising generating a transgenic plant comprising the gene that is associated with the at least one physiological condition. Further provided herein are methods for identifying a modulator of a UVR8-COP1-HY5 UV-B signaling pathway that improves growth in a plant, wherein the gene that is associated with the at least one physiological condition is selected from a group of genes consisting of HY5, CHS, COP1, UVR8, HYH, GPX7, SIG5, CRY3, ELIP1, SWA3, PHYA, FAR1, FHY3, FHY1FHL, MYB111, MYB12, MKP1, PAP1, C4H, MYB4, AtMYB12, AtCHS, and AtC4H. Further provided herein are methods for identifying a modulator of a UVR8-COP1-HY5 UV-B signaling pathway that improves growth in a plant, wherein the gene that is associated with the at least one physiological condition is a modulator of the UVR8-COP1-HY5 UV-B signaling pathway. Further provided herein are methods for identifying a modulator of a UVR8-COP1-HY5 UV-B signaling pathway that improves growth in a plant, wherein the gene that is associated with the at least one physiological condition when expressed activates a downstream regulator of the UVR8-COP1-HY5 UV-B signaling pathway. Further provided herein are methods for identifying a modulator of a UVR8-COP1-HY5 UV-B signaling pathway that improves growth in a plant, wherein the gene that is associated with the at least one physiological condition when expressed increases a gene of the UVR8-COP1-HY5 UV-B signaling pathway. Further provided herein are methods for identifying a modulator of a UVR8-COP1-HY5 UV-B signaling pathway that improves growth in a plant, wherein the gene that is associated with the at least one physiological condition when expressed reduces a suppressor of the UVR8-COP1-HY5 UV-B signaling pathway. Further provided herein are methods for identifying a modulator of a UVR8-COP1-HY5 UV-B signaling pathway that improves growth in a plant, wherein the gene that is associated with the at least one physiological condition is UVR8. Further provided herein are methods for identifying a modulator of a UVR8-COP1-HY5 UV-B signaling pathway that improves growth in a plant, the gene that is associated with the at least one physiological condition is COP1. Further provided herein are methods for identifying a modulator of a UVR8-COP1-HY5 UV-B signaling pathway that improves growth in a plant, wherein the gene that is associated with the at least one physiological condition is HY5. Further provided herein are methods for identifying a modulator of a UVR8-COP1-HY5 UV-B signaling pathway that improves growth in a plant, wherein the gene that is associated with the at least one physiological condition is CHS. Further provided herein are methods for identifying a modulator of a UVR8-COP1-HY5 UV-B signaling pathway that improves growth in a plant, wherein the gene that is associated with the at least one physiological condition is expressed in at least one of seed, seedling, fruit, ovule, carpel, embryo, pericarp, endosperm, pollen, root, leaf, stem, and flower. Further provided herein are methods for identifying a modulator of a UVR8-COP1-HY5 UV-B signaling pathway that improves growth in a plant, wherein the gene that is associated with the at least one physiological condition modulates a downstream responsive gene expressed in at least one of seed, seedling, fruit, ovule, carpel, embryo, pericarp, endosperm, pollen, root, leaf, stem, and flower. Further provided herein are methods for identifying a modulator of a UVR8-COP1-HY5 UV-B signaling pathway that improves growth in a plant, wherein the gene that is associated with the at least one physiological condition when activated increases expression of at least one of flavonoid, anthocyanin, ascorbate acid, and tocopherol. Further provided herein are methods for identifying a modulator of a UVR8-COP1-HY5 UV-B signaling pathway that improves growth in a plant, wherein the gene that is associated with the at least one physiological condition when activated increases expression of flavonoid. Further provided herein are methods for identifying a modulator of a UVR8-COP1-HY5 UV-B signaling pathway that improves growth in a plant, wherein the gene that is associated with the at least one physiological condition when activated increases expression of anthocyanin. Further provided herein are methods for identifying a modulator of a UVR8-COP1-HY5 UV-B signaling pathway that improves growth in a plant, wherein the gene that is associated with the at least one physiological condition modulates expression of a plant hormone selected from the group consisting of auxins, gibberellins, cytokinins, and brassinosteroids. Further provided herein are methods for identifying a modulator of a UVR8-COP1-HY5 UV-B signaling pathway that improves growth in a plant, wherein the gene that is associated with the at least one physiological condition modulates expression of a plant ripening hormone. Further provided herein are methods for identifying a modulator of a UVR8-COP1-HY5 UV-B signaling pathway that improves growth in a plant, wherein the gene that is associated with the at least one physiological condition modulates expression of a seed germination hormone. Further provided herein are methods for identifying a modulator of a UVR8-COP1-HY5 UV-B signaling pathway that improves growth in a plant, wherein improvement of the physiological condition is characterized by an increase in at least one of dry weight, shoot fresh weight, pigment production, radical length, leaf size, and nitrogen index. Further provided herein are methods for identifying a modulator of a UVR8-COP1-HY5 UV-B signaling pathway that improves growth in a plant, wherein the physiological condition is enhanced by at least 5%. Further provided herein are methods for identifying a modulator of a UVR8-COP1-HY5 UV-B signaling pathway that improves growth in a plant, wherein the physiological condition is enhanced by at least 10%. Further provided herein are methods for identifying a modulator of a UVR8-COP1-HY5 UV-B signaling pathway that improves growth in a plant, wherein the physiological condition is enhanced by at least 30%. Further provided herein are methods for identifying a modulator of a UVR8-COP1-HY5 UV-B signaling pathway that improves growth in a plant, wherein the physiological condition is enhanced by at least 50%. Further provided herein are methods for identifying a modulator of a UVR8-COP1-HY5 UV-B signaling pathway that improves growth in a plant, wherein the plant material is selected from the group consisting of lettuce, beans, broccoli, cabbage, carrot, cauliflower, cucumber, melon, onion, peas, peppers, pumpkin, spinach, kale, squash, sweetcorn, corn, maize, tomato, watermelon, alfalfa, canola, cotton, sorghum, soybeans, sugar beets, wheat, rice, and a grass. Further provided herein are methods for identifying a modulator of a UVR8-COP1-HY5 UV-B signaling pathway that improves growth in a plant, wherein the plant material is an indoor plant. Further provided herein are methods for identifying a modulator of a UVR8-COP1-HY5 UV-B signaling pathway that improves growth in a plant, wherein the plant material is an outdoor plant. Further provided herein are methods for identifying a modulator of a UVR8-COP1-HY5 UV-B signaling pathway that improves growth in a plant, wherein the plant material comprises at least one of a seed, a seedling, and a mature plant.

Provided herein are transgenic plants comprising an isolated polynucleotide comprising a nucleic acid sequence encoding a modulator that is responsive to UV-B administration in a plant material of the transgenic plant. In some cases the transgenic plant produces at least one enhanced phenotype in the absence of the supplementary UV-B irradiation, and wherein the at least one enhanced phenotype is selected from the group consisting of increased crop yield, growth rate, hardiness, stress resistance, and pathological resistance when compared to a plant lacking the modulator. Further provided herein are transgenic plants, wherein the modulator is a modulator of the UVR8-COP1-HY5 UV-B signaling pathway. Further provided herein are transgenic plants, wherein the modulator is selected from a group of genes consisting of Hy5, CHS, COP1, UVR8, HYH, GPX7, SIG5, CRY3, ELIP1, SWA3, PHYA, FAR1, FHY3, FHY1FHL, MYB111, MYB12, MKP1, PAP1, C4H, MYB4, AtMYB12, AtCHS, and AtC4H. Further provided herein are transgenic plants, wherein the modulator when expressed activates a downstream regulator of the UVR8-COP1-HY5 UV-B signaling pathway. Further provided herein are transgenic plants, wherein the modulator when expressed increases accumulation of a transcript encoding a member of the UVR8-COP1-HY5 UV-B signaling pathway. Further provided herein are transgenic plants, wherein the modulator when expressed reduces a suppressor of UVR8-COP1-HY5 UV-B signaling pathway. Further provided herein are transgenic plants, wherein the modulator is UVR8. Further provided herein are transgenic plants, wherein the modulator is COP1. Further provided herein are transgenic plants, wherein the modulator is HY5. Further provided herein are transgenic plants, wherein the modulator is CHS. Further provided herein are transgenic plants, further comprising a transgenic tissue-specific promoter. Further provided herein are transgenic plants, wherein the tissue-specific promoter comprises at least one of a fruit, ovule-, carpel-, embryo-, pericarp-, endosperm-, pollen-, root-, leaf-, stem-, and a flower-specific promoter. Further provided herein are transgenic plants, further comprising a polynucleotide for increasing a level of an endogenous plant hormone. Further provided herein are transgenic plants, wherein the plant hormone is selected from the group consisting of auxins, gibberellins, cytokinins, and brassinosteroids. Further provided herein are transgenic plants, further comprising a promoter for expressing a polynucleotide in the presence of a plant hormone. Further provided herein are transgenic plants, wherein the plant hormone is selected from the group consisting of auxins, gibberellins, cytokinins, and brassinosteroids. Further provided herein are transgenic plants, further comprising a promoter specific for expressing a polynucleotide during fruit ripening. Further provided herein are transgenic plants, further comprising a promoter specific for expressing a polynucleotide during seed germination. Further provided herein are transgenic plants, further comprising a constitutive promoter. Further provided herein are transgenic plants, wherein the transgenic plant has improvement of a physiological condition characterized by an increase in at least one of dry weight, shoot fresh weight, pigment production, radical length, leaf size, and nitrogen index. Further provided herein are transgenic plants, wherein the phenotype is enhanced by at least 5%. Further provided herein are transgenic plants, wherein the phenotype is enhanced by at least 10%. Further provided herein are transgenic plants, wherein the phenotype is enhanced by at least 30%. Further provided herein are transgenic plants, wherein the phenotype is enhanced by at least 50%. Further provided herein are transgenic plants, wherein the transgenic plant is selected from the group consisting of lettuce, beans, broccoli, cabbage, carrot, cauliflower, cucumber, melon, onion, peas, peppers, pumpkin, spinach, kale, squash, sweetcorn, corn, maize, tomato, watermelon, alfalfa, canola, cotton, sorghum, soybeans, sugar beets, wheat, rice, grass, and flowering plants. Further provided herein are transgenic plants, wherein the transgenic plant is an indoor plant. Further provided herein are transgenic plants, wherein the transgenic plant is an outdoor plant. Further provided herein are transgenic plants, wherein the plant material comprises at least one of a seed, a seedling, and a plant. Further provided herein are transgenic plants, wherein the transgenic plant is a seed. Further provided herein are transgenic plants, wherein the transgenic plant is grown from a transgenic seed. For each of the embodiments of transgenic plants recited above, also disclosed are similarly transgenic seeds, such as seeds arising from or leading to transgenic plants described above or elsewhere herein.

Provided herein are methods of generating a transgenic plant having at least one of improved plant performance and improved hardiness, comprising transforming a UV-B responsive gene into a wildtype plant cell, wherein the UV-B responsive gene is responsive to light enriched for UV-B in a range of about 281 nm to about 291 nm. Further provided herein are methods of generating a transgenic plant having at least one of improved plant performance and improved hardiness, wherein the improved plant performance is selected from a group consisting of fruit fresh weight, number of fruit harvested, Brix content, fruit width, fruit length, leaf size, leaf surface area, dry weight, nitrogen content, shoot dry weight, shoot fresh weight, root dry weight, vegetable development, yield of fruiting parts, weight of fruiting parts, hardiness, root growth, root architecture, root nodule formation, and seed germination rate. Further provided herein are methods of generating a transgenic plant having at least one of improved plant performance and improved hardiness, wherein the root architecture comprises at least one of nodule formation, root growth, and spatial configuration. Further provided herein are methods of generating a transgenic plant having at least one of improved plant performance and improved hardiness, wherein the improved hardiness is selected from a group consisting of an improved resistance to stress caused by weather damage, an improved resistance to stress caused by sun exposure, an improved resistance to stress caused by disease, and an improved resistance to stress caused by insects. Further provided herein are methods of generating a transgenic plant having at least one of improved plant performance and improved hardiness, wherein the UV-B responsive gene is responsive to UV-B peaking at 286 nm, or to UV-B having a range of wavelengths from 280-290 nm, or from 300-310 nm. Further provided herein are methods of generating a transgenic plant having at least one of improved plant performance and improved hardiness, wherein the UV-B responsive gene is responsive to UV-B having an irradiance up to 1.3×10-4 W cm⁻² s⁻¹. Further provided herein are methods of generating a transgenic plant having at least one of improved plant performance and improved hardiness, wherein the UV-B responsive gene is responsive to UV-B having a dose of no more than 100 kJ m⁻². Further provided herein are methods of generating a transgenic plant having at least one of improved plant performance and improved hardiness, wherein the UV-B responsive gene is selected from a group consisting of HY5, CHS, COP1, UVR8, HYH, GPX7, SIG5, CRY3, ELIP1, SWA3, PHYA, FAR1, FHY3, FHY1FHL, MYB111, MYB12, MKP1, PAP1, C4H, MYB4, AtMYB12, AtCHS, and AtC4H. Further provided herein are methods of generating a transgenic plant having at least one of improved plant performance and improved hardiness, wherein the UV-B responsive gene is a modulator of the UVR8-COP1-HY5 UV-B signaling pathway. Further provided herein are methods of generating a transgenic plant having at least one of improved plant performance and improved hardiness, wherein the UV-B responsive gene is UVR8. Further provided herein are methods of generating a transgenic plant having at least one of improved plant performance and improved hardiness, wherein the UV-B responsive gene is COP1. Further provided herein are methods of generating a transgenic plant having at least one of improved plant performance and improved hardiness, wherein the UV-B responsive gene is HY5. Further provided herein are methods of generating a transgenic plant having at least one of improved plant performance and improved hardiness, wherein the UV-B responsive gene is CHS. Further provided herein are methods of generating a transgenic plant having at least one of improved plant performance and improved hardiness, wherein the at least one of improved plant performance and improved hardiness is enhanced by at least 5%. Further provided herein are methods of generating a transgenic plant having at least one of improved plant performance and improved hardiness, wherein the at least one of improved plant performance and improved hardiness is enhanced by at least 10%. Further provided herein are methods of generating a transgenic plant having at least one of improved plant performance and improved hardiness, wherein the at least one of improved plant performance and improved hardiness is enhanced by at least 30%. Further provided herein are methods of generating a transgenic plant having at least one of improved plant performance and improved hardiness, wherein the plant is selected from the group consisting of lettuce, beans, broccoli, cabbage, carrot, cauliflower, cucumber, melon, onion, peas, peppers, pumpkin, spinach, kale, squash, sweetcorn, corn, maize, tomato, watermelon, alfalfa, canola, cotton, sorghum, soybeans, sugar beets, wheat, rice, and a grass. Further provided herein are methods of generating a transgenic plant having at least one of improved plant performance and improved hardiness, wherein the UV-B responsive gene is mutated. Further provided herein are methods of generating a transgenic plant having at least one of improved plant performance and improved hardiness, wherein the UV-B responsive gene is mutated using methods comprising at least one of CRISPR, zinc finger nucleases, and transcription activator-like effector nucleases. Further provided herein are methods of generating a transgenic plant having at least one of improved plant performance and improved hardiness, wherein the plant cell comprises at least one of a seed cell, a seedling cell, and a mature plant cell.

Provided herein are transgenic plants comprising a UV-B responsive gene, wherein the UV-B responsive gene is responsive to light enriched for UV-B in a range of about 281 nm to about 291 nm. Further provided herein are transgenic plants comprising a UV-B responsive gene, wherein the UV-B responsive gene is mutated. Further provided herein are transgenic plants comprising a UV-B responsive gene, wherein the UV-B responsive gene is mutated using methods comprising at least one of CRISPR, zinc finger nucleases, and transcription activator-like effector nucleases. Further provided herein are transgenic plants comprising a UV-B responsive gene, wherein the UV-B responsive gene is responsive to UV-B peaking at 286 nm. Further provided herein are transgenic plants comprising a UV-B responsive gene, wherein the UV-B responsive gene is responsive to UV-B having an irradiance up to 1.3×10-4 W cm⁻² s⁻¹. Further provided herein are transgenic plants comprising a UV-B responsive gene, wherein the UV-B responsive gene is responsive to UV-B having a dose of no more than 100 kJ m⁻². Further provided herein are transgenic plants comprising a UV-B responsive gene, wherein the UV-B responsive gene is selected from a group consisting of HY5, CHS, COP1, UVR8, HYH, GPX7, SIG5, CRY3, ELIP1, SWA3, PHYA, FAR1, FHY3, FHY1FHL, MYB111, MYB12, MKP1, PAP1, C4H, MYB4, AtMYB12, AtCHS, and AtC4H. Further provided herein are transgenic plants comprising a UV-B responsive gene, wherein the UV-B responsive gene is a modulator of the UVR8-COP1-HY5 UV-B signaling pathway. Further provided herein are transgenic plants comprising a UV-B responsive gene, wherein the UV-B responsive gene is UVR8. Further provided herein are transgenic plants comprising a UV-B responsive gene, wherein the UV-B responsive gene is COP1. Further provided herein are transgenic plants comprising a UV-B responsive gene, wherein the UV-B responsive gene is HY5. Further provided herein are transgenic plants comprising a UV-B responsive gene, wherein the UV-B responsive gene is CHS. Further provided herein are transgenic plants comprising a UV-B responsive gene, wherein the transgenic plant comprises improved plant performance. Further provided herein are transgenic plants comprising a UV-B responsive gene, wherein the improved plant performance is selected from a group consisting of fruit fresh weight, number of fruit harvested, Brix content, fruit width, fruit length, leaf size, leaf surface area, dry weight, nitrogen content, shoot dry weight, shoot fresh weight, root dry weight, vegetable development, yield of fruiting parts, weight of fruiting parts, hardiness, root growth, root architecture, and seed germination rate. Further provided herein are transgenic plants comprising a UV-B responsive gene, wherein the root architecture comprises at least one of nodule formation, root growth, and spatial configuration. Further provided herein are transgenic plants comprising a UV-B responsive gene, wherein the transgenic plant comprises improved hardiness. Further provided herein are transgenic plants comprising a UV-B responsive gene, wherein the improved hardiness is selected from a group consisting of an improved resistance to stress caused by weather damage, an improved resistance to stress caused by sun exposure, an improved resistance to stress caused by disease, and an improved resistance to stress caused by insects. Further provided herein are transgenic plants comprising a UV-B responsive gene, wherein the plant is selected from the group consisting of lettuce, beans, broccoli, cabbage, carrot, cauliflower, cucumber, melon, onion, peas, peppers, pumpkin, spinach, kale, squash, sweetcorn, corn, maize, tomato, watermelon, alfalfa, canola, cotton, sorghum, soybeans, sugar beets, wheat, rice, and a grass. For each of the transgenic plants recited or contemplated herein, similarly is disclosed a seed harboring a transgenic event such as that disclosed above or elsewhere herein.

Provided herein are a methods of reducing environmental impact of growing a crop, comprising the steps of: sowing a seed comprising a UV-B responsive gene, wherein the UV-B responsive gene is responsive to light enriched for UV-B in a range of about 281 nm to about 291 nm; sowing the seed; providing no more than at least one of a standard fertilizer regimen, a standard pesticide regimen, a standard herbicide regimen, and a standard insecticide regimen; and harvesting the crop from said seed, wherein a crop yield of the crop from said seed is at least 5% greater than a standard yield.

Provided herein are transgenic seeds comprising an isolated polynucleotide comprising a nucleic acid sequence encoding a modulator that is responsive to UV-B administration, wherein the transgenic seed produces at least one enhanced phenotype in the absence of the supplementary UV-B irradiation, and wherein the at least one enhanced phenotype is selected from the group consisting of increased crop yield, growth rate, hardiness, stress resistance, and pathological resistance when compared to a seed lacking the modulator.

### INCORPORATION BY REFERENCE

All publications, patents, and patent applications mentioned in this specification are herein incorporated by reference to the same extent as if each individual publication, patent, or patent application was specifically and individually indicated to be incorporated by reference in their entireties.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts washing seeds under cold water.

FIG. 2 illustrates distribution of seeds in a series of dishes.

FIG. 3 illustrates arrangement of seeds and the light source.

FIG. 4 depicts connecting the control system to wifi and zigbee modules.

FIG. 5 illustrates an exemplary sowing key.

FIG. 6 illustrates an exemplary randomized 12×12 sowing key.

FIG. 7 illustrates various components of an exemplary computer system according to various embodiments of the present disclosure.

### DETAILED DESCRIPTION

Disclosed herein are methods and compositions for the identification of genes, transcripts and proteins that regulate UV-B mediated plant responses. Also disclosed herein are transgenic plants that exhibit the beneficial effects of seed or seedling UV-B treatment in the absence of such treatment.

UV-B treatment of seeds is observed to increase both plant growth and plant hardiness. This is in contrast to most plant treatments, which result in a trade-off of growth for hardiness, and which often result in plants that are either slow growing or vulnerable to plant stresses. Thus, identification of the UV-B responsive pathways that mediate the dual promotion of plant growth and plant hardiness in the face of a biotic or abiotic stress is of clear benefit to development of more reliably high-yielding lines.

To identify pathways involved in the UV-B mediated improvements to growth and hardiness, seeds are treated with UV-B and monitored for the effect on growth and hardiness. It is observed that plants growing from treated seeds exhibit a faster growth rate and a higher degree of resistance to biotic and abiotic stresses.

RNA is isolated from treated seeds and compared to RNA from untreated control seeds at a comparable time after stratification or after germination. Transcripts that differentially accumulate in plants derived from treated seeds and that exhibit the joint phenotypes of faster growth and increased hardiness are obtained and assessed for their potential relevance as mediators of the UV-B response.

Alternately or in combination, protein accumulation levels or protein activities are measured for treated and untreated lines. Differences between treated and untreated lines in their protein accumulation levels or activities are assessed, and the genetic factors encoding these proteins are identified.

Transgenic lines are generated that mimic the accumulation levels of differentially accumulating transcripts, proteins or protein activities are generated. In these lines, untreated plants exhibit transcript accumulation levels, or related protein accumulation levels, or related protein activity levels, that are comparable to those of plants derived from UV-B treated seedlings. Such transgenic plants are assayed for their growth rate and hardiness, so as to identify transcripts, proteins or protein activities which, when altered so as to mimic treated line levels, cause the transgenic plants to recapitulate the UV-B treatment associated phenotypes.

Often, transcripts are identified that are implicated in the UVR8-COP1-HY5 UV-B signaling pathway. Accordingly, the disclosure herein focuses on said pathway as an example. However, focus on the UVR8-COP1-HY5 UV-B pathway is not to suggest that other pathways are not also implicated in the UV-B response. Accordingly, disclosed herein are transgenic plants that are perturbed so as to recapitulate UV-B treatment. In some cases this comprises mutagenesis such that the UVR8-COP1-HY5 UV-B pathway is altered such that plants exhibit UV-B treatment phenotypes in the absence of UV-B. However, alternate pathways are implicated in UV-B responses and the disclosure herein, and the transgenic plants contemplated herein are not limited to plants that exhibit a perturbation in the UVR8-COP1-HY5 UV-B signaling pathway.

Transgenic plants recapitulating UV-B administration phenotypes include plants that overexpress or over-accumulate a transcript implicated in the UV-B mediated response, plants that under-express or under-accumulate a transcript implicated in the UV-B mediated response, or that over or under-accumulate a related protein or protein activity. In some cases, transgenic plants are transformed so as to constitutively or in a tissue-specific manner overexpress a transcript of interest, silence or reduce the accumulation level of a transcript of interest, for example using double-stranded RNA, RNAi, shRNA, miRNA or other RNA-mediated post-transcriptional mediation of RNA accumulation levels. Some plants are modified so as to mutate the coding region or related sequence regulating expression of a protein of interest, such as by site-directed or random insertional mutagenesis. In some cases coding regions are mutated so as to eliminate a translation start codon, introduce a frame-shift in a coding region, truncate a protein or introduce an in frame or out of frame deletion in a region of interest, or introduce a missense mutation in a coding region such that the encoded protein demonstrates altered catalytic core residues or an altered residue at a regulatory site, so as to mimic constitutive phosphorylation or constitutively absent phosphorylation.

Unless defined otherwise, all technical and scientific terms used herein have the same meaning as is commonly understood by one of skill in the art to which this disclosure belongs. All patents and publications referred to herein are incorporated by reference.

As used in the specification and claims, the singular form “a”, “an” and “the” includes plural references unless the context clearly dictates otherwise.

As used herein the term “at least one of” in context of a list includes single members of the list, combinations of the members of the list, or to all of the members of the list.

As used herein, the term “subject” generally refers to a biological entity containing expressed genetic materials. The biological entity can be a plant including, e.g., monocotyledons, dicotyledons, gymnosperms (linear and speculate). The subject can be tissues, cells and their progeny of a biological entity obtained in vivo or cultured in vitro.

The term “including” is used to mean “including but not limited to.” “Including” and “including but not limited to” are used interchangeably.

The term “in vivo” refers to an event that takes places in a subject's body.

The term “in vitro” refers to an event that takes places outside of a subject's body. For example, an in vitro assay encompasses any assay run outside of a subject assay. In vitro assays encompass cell-based assays in which cells are lysed or are removed from their multicellular environment.

The term “light intensity” refers herein to measurement of light described herein including but not limited to radiant intensity, luminous intensity, irradiance, radiance, intensity, brightness, luminance, photometry, and radiometry.

The term “radiant intensity” refers to a radiometric quantity measured in watts per steradian (W/sr).

The term “luminous intensity” refers to a photometric quantity measured in lumens per steradian (lm/sr), or candela (cd).

The term “irradiance” refers to a radiometric quantity and may be measured in watts per meter squared (W/m²).

The term “radiance” refers to intensity (W·sr⁻¹·m⁻²).

The term “luminance” refers to the photometric equivalent of radiance (lm·sr⁻¹·m⁻²).

The term “photometry” refers to measurement of light, in terms of its perceived brightness to the human eye.

The term “brightness” refers to the subjective perception elicited by the luminance of a source.

The term “photomorphogenesis” refers to light-mediated development of a plant, where plant growth patterns respond to the light spectrum. Photomorphogenesis is a separate developmental or growing process from photosynthesis. Phytochromes, cryptochromes, and phototropins are photochromic sensory receptors that restrict the photomorphogenic effect of light. UVR8 is the only known photoreceptor that specifically perceives UV-B, while other receptors are involved in UV-A, blue, and red portions of the electromagnetic spectrum. Stages of plant development where photomorphogenesis occurs include but not limited to seed germination, seedling development, and the switch from the vegetative to the flowering stage.

The term “nucleic acid” refers to DNA molecules (e.g., cDNA or genomic DNA), RNA molecules (e.g., mRNA), DNA-RNA hybrids, and analogs of the DNA or RNA generated using nucleotide analogs. The nucleic acid molecule can be a nucleotide, oligonucleotide, double-stranded DNA, single-stranded DNA, multi-stranded DNA, complementary DNA, genomic DNA, non-coding DNA, messenger RNA (mRNA), microRNA (miRNA), small nucleolar RNA (snoRNA), ribosomal RNA (rRNA), transfer RNA (tRNA), small interfering RNA (siRNA), heterogeneous nuclear RNAs (hnRNA), or small hairpin RNA (shRNA).

As used herein, a “profile” of a transcriptome or portion of a transcriptome can refer to any sequencing or gene expression information concerning the transcriptome or portion thereof. This information can be either qualitative (e.g., presence or absence) or quantitative (e.g., levels or mRNA copy numbers). In some embodiments, a profile can indicate a lack of expression of one or more genes.

The term “cDNA library” refers to a collection of complementary DNA (cDNA) fragments. A cDNA library may be generated from the transcriptome of a single cell or from a plurality of single cells. cDNA is produced from mRNA found in a cell and therefore reflects those genes that have been transcribed for subsequent protein expression.

As used herein, a “primer” is a polynucleotide that hybridizes to a target or template that may be present in a sample of interest. After hybridization, the primer promotes the polymerization of a polynucleotide complementary to the target, for example in a reverse transcription or amplification reaction.

The term “trans-activating crRNA tracrRNA’” refers to a small trans-encoded RNA.

The term “crRNA” refers to CRISPR RNA.

The term “chimera cr/tracrRNA hybrid” refers to a duplex of trancrRNA and crRNA. TracrRNA is complementary to and base pairs with a pre-crRNA forming an RNA duplex. This is cleaved by RNase III, an RNA-specific ribonuclease, to form a crRNA/tracrRNA hybrid. This hybrid acts as a guide for the endonuclease Cas9.

As described herein, the term “gene expression” or “gene expression profile” are used interchangeably herein. These terms refers to the process by which information from a gene is used in the synthesis of a functional gene product such as proteins. In non-protein coding genes such as transfer RNA (tRNA) or small nuclear RNA (snRNA) genes, the product can be a functional RNA. Gene expression process may be modulated, including the transcription, RNA splicing, translation, and post-translational modification of a protein. Gene regulation gives the cell control over structure and function, and is the basis for cellular differentiation, morphogenesis and the versatility and adaptability of any organism. Gene regulation may also serve as a substrate for evolutionary change, since control of the timing, location, and amount of gene expression can have a profound effect on the functions (actions) of the gene in a cell or in a multicellular organism. Gene expression may also give rise to the phenotype (e.g., observable traits) of an organism. Such phenotypes are often expressed by the synthesis of proteins that control the organism's shape, or that act as enzymes catalyzing specific metabolic pathways characterizing the organism. Regulation, modulation, or manipulation of gene expression may affect an organism's development.

As used herein, the term “suppress” as referred to a biologically active agent refers to the agent's ability to reduce the target activity as compared to off-target activity, via direct or indirect interaction with the target.

As used herein, the term “genetic disorder” refers a genetic problem caused by one or more abnormalities in the genome. The abnormalities can be at the DNA level, such as a gene mutation, duplication, copy number variation, single nucleotide polymorphism (SNP), insertion, deletion, point mutation, substitution, insertion, deletion, rearrangement, de novo mutation, nonsense mutation, missense mutation, silent mutation, frameshift mutation, amplification, chromosomal translocation, interstitial deletion, chromosomal inversion, loss of heterozygosity, loss of function mutation, gain of function mutation, dominant negative mutation, or lethal mutation. The abnormalities can be post-translational modifications, or protein degradations.

The term “mutation”, as used herein, generally refers to a change of the nucleotide sequence of a genome as compared to a reference. Mutations can involve large sections of DNA (e.g., copy number variation). Mutations can involve whole chromosomes (e.g., aneuploidy). Mutations can involve small sections of DNA. Examples of mutations involving small sections of DNA include, e.g., point mutations or single nucleotide polymorphisms, multiple nucleotide polymorphisms, insertions (e.g., insertion of one or more nucleotides at a locus), multiple nucleotide changes, deletions (e.g., deletion of one or more nucleotides at a locus), and inversions (e.g., reversal of a sequence of one or more nucleotides).

The term “genotype” or “genotyping”, as used herein, generally refers to a process of determining differences in the genetic make-up (genotype) of an individual by examining the individual's DNA sequence using biological assays and comparing it to another individual's sequence or a reference sequence.

As described herein, “a modulator” refers to a compound or an agent which modulates the activity of one or more cellular proteins. A modulator may augment or increase (e.g., an agonist), or suppress or reduce (e.g., antagonist) the activity of a protein. As a non-limiting example, a modulator can be a compound, a synthetic compound, a small molecule, a macromolecule, a nanoparticle, a protein, a plant extract, amino acids, a peptide, nucleic acids, nucleotides, a deoxyribonucleic acid (DNA), a complementary DNA (cDNA), a genomic DNA (gDNA), a mitochondrial DNA, a ribonucleic acid (RNA), a messenger RNA, a small RNA, a short RNA, ribosomal RNA, non-coding RNA, small nuclear ribonucleic acids (snRNA), U-RNA, or mitochondrial RNA.

The term “about” a value refers to a plus or minus 10% of the indicated value. For example, about 50% can be interpreted as 45%-55%. The term “about” a range refers to 10% less than the lowest value of the range to 10% above the largest vale of the range.

The term “about” as used herein in reference to wavelength refers to 1% below the number to 1% above the number.

The term “transgenic plant” refers to a whole plant as well as to seed, seedling, fruit, leaves, roots, other plant tissue, plant cells, protoplasts, callus, immature plant, mature plant, or any other plant material, and progeny thereof. Transgenic plants are plants which contain isolated polynucleotides or polypeptides which are introduced into plants, for example by transformation, and are stably integrated into at least one cell genome so as to be replicated upon meiotic or mitotic division of the cell.

The term “plant material” refers a seed, seedling, immature plant, mature plant, fruit, leaves, roots, cuttings, runners, or any other plant material and progeny thereof.

As described herein, the term “transformation” refers to introducing a nucleotide sequence in a plant in a manner to cause stable or transient expression of the sequence. This may be achieved by transfection with viral vectors, transformation with plasmid vectors or introduction of naked DNA by electroporation, lipofection, particle gun acceleration or other approach known to one of skill in the art.

The terms “polynucleotides,” “nucleic acid,” “nucleic acid molecules,” “nucleotides” and “oligonucleotides” can be used interchangeably. They can refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA(tRNA), ribosomal RNA (rRNA), Small nuclear ribonucleic acid (snRNA), U-RNA, non-coding RNA (ncRNA), non-protein-coding RNA (npcRNA), non-messenger RNA (nmRNA), functional RNA (fRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

A “fragment” or “a partial thereof”, as applies to polypeptides, is a portion of a polypeptide that is recognizably identified as part of its source polypeptide to the exclusion of non-origin polypeptides. In some cases a fragment can perform at least one biological activity of the intact polypeptide in substantially the same manner as the intact polypeptide does. A fragment may vary in size from as few as 9 amino acids to the length of the intact polypeptide, but can be at least 30 amino acids in length. The amino acids selected from the intact polypeptide need not be consecutive. In reference to nucleotide sequences “a fragment” refers to any sequence of at least 5, 10, 15, 20, 30, 40, 50, 60, 70, 80, 90, 100, or more nucleotides.

The term “isolated polynucleotide” refers to a nucleotide sequence that is not in its native state, for example, when it is separated from nucleotide sequences with which it typically is in proximity in a genome or is next to other nucleotide sequences with which it typically is not. The nucleotide sequence may comprise a coding sequence or fragments thereof, promoters, introns, enhancer regions, polyadenylation sites, translation initiation sites, reporter genes, selectable markers or the like. The polynucleotide may be single stranded or double stranded DNA or RNA. The polynucleotide may be a genomic or processed nucleotide sequence (such as cDNA or mRNA). The nucleotide sequence may be in a sense or antisense orientation

An “isolated polypeptide” is a polypeptide that is more enriched than the polypeptide in its natural state in a cell, e.g., at least 5%, 10%, 15%, 20%, 30%, 40% 50% 60%, 70%, 80%, 90%, 99%, or more enriched.

A “homologous sequence” refers to a sequence having sequence identity inferred from common descent. Often, a homologous sequence has a sequence has a certain degree of sequence identity with a second sequence after alignment as determined by using sequence analysis programs for database searching and sequence comparison available from the Wisconsin Package, such as BLAST, FASTA, PILEUP, FINDPATTERNS or the like (GCG, Madison, Wisc.). Public sequence databases such as GENBANK, EMBL, Swiss-Prot and PIR or private sequence databases such as PhytoSeq (Incyte Pharmaceuticals, Palo Alto, Calif) may be searched. Homologous sequences when expressed in a plant may cause essentially the same effect, for example, two polypeptides having essentially the same effect on the hardiness of the stem, leafs, or a whole of the plant, the yield of a plant, or the stress resistance of a plant.

Often, homologous sequences from different plant species are identified as follows. A first plant sequence is used to query a sequence dataset for a second plant species. The strongest hit, or a group of strongest hits from the second plant species are identified as putative homologues of the first plant sequence. This hit or hits are used to query against a sequence dataset of the first plant species. If the second plant species returns the original sequence or a closely related sequence as its strongest hit, then the second sequence is inferred to be a homologue of the first plant sequence.

Homologues, particularly among closely related species, are often highly similar to one another. However, even when homologues do not demonstrate a high degree of similarity across their entire lengths, homologues often demonstrate sufficient sequence or structural conservation such that a homologue from one species is able to functionally substitute for a lost functional homologue of a second species. That is, homologues are often able to complement the phenotype caused by a defect in a homologous gene in a second species. Similarly, a phenotype cause by over, under, or misexpression, or other alteration of a homologue in one species is expected to be observed if a similar alteration is made in the homologous gene of a second species.

The term “seed” refers to an embryonic plant enclosed in a protective outer covering. The formation of the seed is part of the process of reproduction in seed plants, the spermatophytes, including the gymnosperm and angiosperm plants. Seeds are the product of the ripened ovule, after fertilization by pollen and some growth within the mother plant. The embryo is developed from the zygote and the seed coat from the integuments of the ovule. Seeds can be sown in shell, husk, or a tuber.

The term “seed germination” refers to a process by which a seed embryo develops into a seedling. It involves the reactivation of the metabolic pathways that lead to growth and the emergence of the radicle or seed root and plumule or shoot. In general, seed germination occurs in three phases: water imbibition, lag phase, and radicle emergence. Seed germination may be affected by environmental conditions including, but not limited to, water, oxygen, temperature, and light.

The terms “stratify,” “imbibe”, “imbibition”, “prime”, or “priming” are used interchangeably herein. These terms refer to immersing seed in water in order for the embryo to imbibe or soak up water, which causes the embryo to well thereby splitting the seed coat. The nature of the seed coat may determine how rapidly water can penetrate and subsequently initiate germination. The rate of imbibition can be dependent on the permeability of the seed coat, amount of water in the environment and the area of contact the seed has to the source of water

Seedling establishment refers to the emergence of the seedling above the soil surface.

The term “seed dormancy” refers to a circumstance when a seed retains viability but does not germinate. Often, dormancy is measured by counting when a seed or a number of seeds fail to germinate under environmental conditions optimal for germination, normally when the environment is at a suitable temperature with proper soil moisture. Seed dormancy can be a state of the seed as a result of conditions within the seed that prevent germination. Seed dormancy can be affected by external environment. For instance, induced dormancy, enforced dormancy or seed quiescence occurs when a seed fails to germinate because the external environmental conditions are inappropriate for germination, mostly in response to conditions being too dark or light, too cold or hot, or too dry. Seed dormancy can be exogenous, endogenous, combinational, secondary, morphological, physiological, morphophysiological, physical, chemical, photodormancy, and thermodormancy.

The term “photodormancy” or light sensitivity may affect germination of some seeds. For example, photoblastic seeds need a period of darkness or light to germinate. In species with thin seed coats, light may be able to penetrate into the dormant embryo. The presence of light or the absence of light may trigger the germination process, inhibiting germination in some seeds buried too deeply or in others not buried in the soil.

A “fruit” refers to any seed-containing organ of a plant.

The term “plant performance” as used herein refers to improving at least one of resilience and growth. Resilience, as used herein refers to biotic or abiotic environmental stress, which can impact the seed, the seedling, the resulting plant, the resultant crop before or after harvesting. “Growth” generally refers to performance in the absence of an abiotic or biotic stress, such as performance under healthy or ‘best case scenario’ growth conditions. One observes that, depending upon growth conditions, both increase resilience and improvements in growth can result in increases in yield, depending upon growth conditions. One observes that improving both growth and resilience has the effect of improving yield of harvestable crop material relative plants resulting from untreated seeds independent of growth conditions. Plant performance also refers in some cases to improving quality of harvestable crop material, such that plant value is increased per unit yield even if yield, more coarsely defined, is unaffected. Some non-limiting examples of improved stress resilience are improved drought resistance, salinity stress, transplantation shock, long-term hardiness, high visible light stress, insect pest stress, fungal or bacterial stress, or other disease-related stress. The term “crop productivity” may in some cases be used interchangeably with “plant performance.”

The term “long-term hardiness” as used herein refers to the ability of a plant to withstand one or more stresses during crop production and to allow improved yield and/or quality of the plant at harvesting. Some non-limiting examples of how improved yield is measured include weight of harvestable crop material, such as lettuce leaves, soybeans, tomato fruit, in comparison to harvestable crop material where the seeds for sowing were not treated with UV-B. Other examples of how improved yield are measured include fresh shoot weight or whole plant dry weight, improved germination of seeds resulting from the treatment method, and improved water use efficiency of the resulting plant. In some cases, improved quality is assessed as a quantitative or qualitative assessment of at least one of a lack of blemishes on the crop (either internal or on the surface, typically from insects), improved shelf life, improved resistance to bruising or other post-harvest handling, lack of deformities, lack of irregular shapes, lack of irregular sizes, improved taste, size, shape, color, and texture. An advantage of the present disclosure is that both stress resilience and plant yield were observed (often these traits can work in an inverse relationship, where resilience is achieved at the cost of yield as seen with UV-C treatment).

A “promoter” is a polynucleotide sequence that controls the expression of a gene and is operably linked to a gene of interest. Constitutive promoters express a gene in all tissues, at all times and under all conditions. Specific promoters (or active promoters) may cause preferential (for example higher levels of expression in specific tissue, but not to the exclusion of lower expression levels in other tissue) or selective expression (for example levels of expression occur only under specific conditions to the exclusion of other expression) in particular tissue, at different developmental stages, or in response to endogenous or exogenous compounds. Expression levels of a transcript may be detected by Northern, real time polymerase chain reaction (RT-PCR), RNA-seq, quantitative-PCR (Q-PCR), gene sequencing, or gene expression array systems. A promoter may be a polynucleotide sequence comprising an endogenous promoter of the gene of interest. In some cases, a promoter is a polynucleotide sequence comprising a binding site for polymerase, a binding site of a transcription factor that activates or suppresses transcription of the gene of interest.

### Overview

Plants are constantly challenged by harsh environmental conditions. Variations in environmental conditions influence plant growth and plant performance. In order to adapt to these environmental conditions and their changes, most plants furnish complicated signaling systems that regulate gene expression which determines general plant performance. Light is one of the most important environmental factors for plant growth and development throughout its life cycle. To withstand the environmental changes of light species, brightness and intensity, plants furnish the UVR8/COP1/HY5 cascade that links several diverse classes of photoreceptors. The UVR8/COP1/HY5 cascade includes UV-B responsive genes, which are either activated or suppressed in response to UV-B exposure. Although UV-B generally damages DNA, inhibits photosynthesis and arrests cell cycles, defined photon energy in the UV-B spectrum and defined dose and duration of UV-B exposure may improve plant performance, growth and resistance to biotic and/or abiotic stresses (see e.g., International Patent Application No. PCT/NZ2015/050153, published as WO2016/043605 on Mar. 24, 2016, which is incorporated herein by reference in its entirety). Provided herein are systems and methods for identifying polynucleotides or partial thereof responsive to UV-B exposure. Expression of these polynucleotides or partial thereof may modulate the UVR8/COP1/HY5 cascade, thereby enhancing plant agronomic traits such as enhanced plant performance, plant growth, plant hardiness, biotic stress resistance, and abiotic stress resistance. The disclosed systems and methods also provide for generating stable transgenic plants that express the desired agronomic traits.

Aspects of the application relate to systems and methods that enhance plant performance. The systems and methods provide for enhancing plant performance via manipulating a UVR8/COP1/HY5 pathway modulator. The manipulation may involve exposure to a defined UV-B spectrum at a controlled dosage and time period. The plant may be exposed to the disclosed UV-B spectrum and dosage at a variety of stages including, but not limited to, seeds stage, germinating stage, immature plant, and mature plant. In some embodiments, samples from the UV-B exposed plant or seed are collected for nucleotide extraction, amplification, sequencing, or constructing microarray to identify nucleotides encoding genes responsive to UV-B treatment. In some embodiments, stable transgenic plants of the identified genes are generated. The transgenic plants may be selected for a phenotype, e.g., any of the desired agronomic trait described herein.

A number of plant types are consistent with the disclosure herein. Plants relating to the present disclosure can be indoor plants. The plants can be outdoor plants. The plants can be fruit plants. The plant can be flowering plant. A plant consistent with the present disclosure may be edible. The plant may be a garden vegetable or herb. As a non-limiting example, the plant can be lettuce, beans, broccoli, cabbage, carrot, cauliflower, cucumber, melon, onion, peas, peppers, pumpkin, spinach, kale, squash, sweetcorn, corn, maize, tomato, watermelon, alfalfa, canola, cotton, sorghum, soybeans, sugar beets, wheat, rice, or a grass. The plants may be a gymnosperm or, in particular, an angiosperm. The plants may be in any family, such as the Asteraceae, Brassicaceae, Poaceae, Solanaceae, Fabaceae, Labiaceae, Rosaceae, or other family.

In various aspects, disclosed herein are systems and methods for enhancing plant performance by irradiating a plant with a defined photon energy, wavelength, dose and duration of light source. Exposure of the plant to the systems and methods disclosed herein may improve at least one physiological condition such as crop yield, and plant performance under biotic stress and abiotic stress. The biotic stress can be caused by infections from yeast, fungi, bacteria, insects, and parasite. The abiotic stress can be caused by inanimate components of the environment associated with climatic, edaphic and physiographic factors that substantially limit plant growth and survival. Non-limiting examples of abiotic stress include drought, salinity, non-optimal temperatures, poor soil nutrition, herbicides, and pesticides. The light source may have an enriched wavelength in a range between 280-310 nm. The light source may have an enriched wavelength at about 280 nm. The light source may be enriched UV-B. The light source may have photon energy in a range between 3.94-4.43 eV. The light source may have photon energy in a range between 0.5-0.8 aJ. The light source may have irradiance in a range between 4×10⁻⁵ to 1.3×10⁻⁴ wcms⁻¹. The light treatment may be at a dose of 13 kJ m⁻². The light treatment may be at a dose of 100 kJ m⁻². The light treatment may be at a dose between 13 kJ m⁻² to 100 kJ m⁻². The plant may be treated with the defined photo energy and dose of light for at least 1 h. The plant may be treated with the defined photo energy and dose of light for a sufficient time to elicit change of at least one physiological condition in the treated plant. The defined and controlled light may be provided by a LED source. The plant may be a seed. The plant may be an immature plant. The plant may be a mature plant. The seed may be imbibed with water and primed prior to sowing. In some embodiments, the systems and methods comprises exposing the plant to the defined and controlled light source and visible light (e.g. red/blue) simultaneously or sequentially. Treatment of a plant with the systems and methods disclosed herein may activate a modulator of the UVR8-COP1-HY5 UV-B signaling pathway and may increase output of the UVR8-COP1-HY5 UV-B signaling pathway. In some cases, activation of the UVR8-COP1-HY5 UV-B signaling pathway reduces output of the UVR8-COP1-HY5 UV-B signaling pathway. In general, activation of the UVR8-COP1-HY5 UV-B signaling pathway modulator may improve at least one physiological condition in the plant such as improved crop yield, growth rate, hardiness, stress resistance (biotic or abiotic), pathological resistance, fruit size, fruit taste, fruit production, flower production, fruit ripening, higher seed germination rate, and/or pigmentation. In some aspects, the systems and methods further comprising identifying the UVR8-COP1-HY5 UV-B signaling pathway modulator and generating transgenic plants of the identified gene. The identified gene may be a novel gene in the UVR8-COP1-HY5 UV-B signaling pathway. The identified gene may be a gene in the UVR8-COP1-HY5 UV-B signaling pathway that has been well-studied. In some cases, the function of the identified gene in enhancing plant performance in response to the instant UV-B treatment is novel to the field. The transgenic plants may be subjected for selective breeding or genetic modification approach to activate beneficial responses from seed treatment. The transgenic plant and offspring may not require additional UV treatment as described herein to achieve the desired plant performance. The transgenic plant and offspring may be subjected to additional UV treatment as described herein to further enhance plant performance.

In some instances, when UV-B is co-administered with light of another wavelength, UV-B is enriched as compared to the light of another wavelength. In some instances, UV-B is enriched at least or about 15%, 20%, 25%, 30%, 35%, 40%, 45%, 50%, 60%, 70%, 80%, 90%, 100%, 125%, 150%, 175%, 200%, 225%, 250%, 275%, 300%, or more than 300% more than the light of another wavelength. In some instances, UV-B is supplemented. In some instances, UV-B is the predominant wavelength during light administration. In some instances, UV-B comprises at least or about 15%, 20%, 25%, 30%, 35%, 40%, 45%, 50%, 60%, 70%, 80%, 90%, 100% of light for light administration.

Disclosed herein are systems and methods for identifying a modulator of UVR8-COP1-HY5 UV-B signaling pathway. Some such systems and methods comprising irradiating a plant with enriched UV-B at a wavelength of from 280-320 nm for a period of time. In some cases, the enriched UV-B has a wavelength about 280 nm, for example 280, 281, 282, 283, 284, 285, 286, 287, 288, 289, 290, 291, 292, 293, 294, 295, 296, 297, 298, 299, 300, or greater than 300. In some cases, the enriched UV-B has a wavelength of 280 nm. The UV-B supply may be controlled at an effective dose and time exposure for producing change in a physiological condition in the treated plant. The light treatment may be at a dose of 13 kJ m⁻². The light treatment may be at a dose of 100 kJ m⁻². The light treatment may be at a dose between 13 kJ m⁻² to 100 kJ m⁻². The UV-B exposure can be at least 1 h. The UV-B exposure can be provided continuously or intermittently for an effective duration of time. The plant can be a seed. The plant can be an immature plant. The plant can be a mature plant. The plant may be inspected for improvement in at least one physiological condition including, but not limited to, crop yield, growth rate, hardiness, stress resistance (biotic or abiotic), and pathological resistance. Improvement of a physiological condition is evaluated by comparing performance of a plant that has been irradiated with the disclosed UV-B wavelength and dose. A polynucleotide that is associated with the enhanced physiological condition is selected and identified. In some cases, the polynucleotide encodes a gene in the UVR8-COP1-HY5 UV-B signaling pathway. In some cases, the polynucleotide is novel in the UVR8-COP1-HY5 UV-B signaling pathway. The polynucleotide can be a gene or a fragment thereof encoding UVR8. The polynucleotide can be a gene or a fragment thereof encoding COP1. The polynucleotide can be a gene or a fragment thereof encoding HY5. The polynucleotide can be a gene or a fragment thereof encoding CHS. In some cases, more than one polynucleotide or gene is involved in determining an enhanced plant performance. In some cases, a polynucleotide or gene is in a signaling pathway that interacts with the UVR8-COP1-HY5 UV-B signaling pathway. In some cases, a polynucleotide or gene is in a signaling pathway independent of the UVR8-COP1-HY5 UV-B signaling pathway.

In some embodiments, the polynucleotides that are responsible for UV-B induced resistance to biotic, abiotic and/or enhanced plant performance are genes relate to the UVR8-COP1-HY5 UV-B signaling pathway. A variety of UVR8-COP1-HY5 UV-B signaling pathway genes have been reported, for example in Tohge et. al., 2011. Transcriptional and metabolic programs following exposure of plants to UV-B irradiation. Plants Signaling & Behavior 6:12, 1987-1992, which is incorporated by reference hereby in its entirety. Example genes in the UVR8-COP1-HY5 UV-B signaling pathway include HY5, CHS, COP1, UVR8, HYH, GPX7, SIG5, CRY3, ELIP1, SWA3, PHYA, FAR1, FHY3, FHY1FHL, MYB111, MYB12, MKP1, PAP1, C4H, MYB4, AtMYB12, AtCHS, and AtC4H.

In various embodiments, the polynucleotide identified herein is associated with the at least one physiological condition and is expressed at least in fruit, ovule, carpel, embryo, pericarp, endosperm, pollen, root, leaf, stem, flower, or combinations thereof. The polynucleotide may modulate a downstream responsive gene expressed at least in fruit, ovule, carpel, embryo, pericarp, endosperm, pollen, root, leaf, stem, flower, or combinations thereof. The polynucleotide may be a transcription factor that regulates gene expression. The polynucleotide may encode a functional molecule such as a pigment, a hormone, or a signaling receptor. The polynucleotide may be expressed in more than one location of a plant at a particular stage of the plant life cycle. For example, the polynucleotide may be expressed in the embryo and pericarp throughout the life cycle. As another example, the polynucleotide may first expresses in the embryo in a seed during embryonic stage and subsequently expresses in the pericarp in the fruit of a mature plant. As another example, the polynucleotide may be expressed exclusively in the embryo prior to or during seed germination.

The polynucleotide may encode a protein or a partial fragment thereof that is responsible for production of at least one plant pigment such as flavonoid, anthocyanin, ascorbate acid, vitamin, tocopherol, carotenoid or combinations thereof. Activation of the gene may increase production of flavonoid. Activation of the gene may increase production of anthocyanin. Activation of the gene may increase production of ascorbate acid. Activation of the gene may increase production of tocopherol. Activation of the gene may increase production of carotenoid. In some cases, accumulation of said pigment is used as a reporter in an assay for activity associated with overexpression, underexpression or misexpression of said at least one gene or partial fragment thereof.

The polynucleotide may encode a protein or a part thereof that is responsible for production or secretion of at least one plant hormone such as auxins, gibberellins, cytokinins, brassinosteroids, or combinations thereof. The gene may be responsible for production, degradation, clearance or secretion of auxins, such as through the production of an auxin precursor molecule. The protein may be responsible for production, degradation, clearance or secretion of gibberellins, such as through the production of a gibberellin precursor molecule. The gene may be responsible for production, degradation, clearance or secretion of cytokinins, such as through the production of a cytokinin precursor molecule. The gene may be responsible for production, degradation, clearance or secretion of brassinosteroids, such as through the production of a brassinosteroid precursor molecule. In some cases, the UVR8-COP1-HY5 UV-B signaling pathway modulator identified herein regulates expression or function of a plant ripening hormone. In some cases, the UVR8-COP1-HY5 UV-B signaling pathway modulator identified herein regulates expression or function of a seed germinating hormone. In some cases an alternate signaling pathway is involved in the response.

Manipulation on the UVR8-COP1-HY5 UV-B signaling pathway or other signaling pathway modulator identified herein in a plant may improve plant performance. Non-limiting example of plant performance include increase dry weight, shoot fresh weight, pigment production, radical length, leaf size, nitrogen index, and combinations thereof. In some instances, plant performance is enhanced root growth or root architecture. Root architecture may comprise improved nodule formation. In some instances, root architecture comprises deeper root growth. In some instances, root architecture comprises improved spatial configuration of the roots. The improvement may be evaluated by comparing corresponding performance in a plant treated with the instant UV-B treatment and a plant that has not been treated with the instant UV-B treatment. UV-B treatment using the systems and methods disclosed herein may improve plant performance by a significant percentage when compared to the counterpart plants that have not been treated with the UV-B regimen disclosed herein. The plant performance may be increased by about 5-100, 10-90, 20-80, 30-70, 40-60, 50-95, 65-85, or 75-95%. The plant performance may be increased by at least 5, 10, 15, 20, 25, 30, 35, 40, 45, 50, 55, 60, 65, 70, 76, 80, 85, 90, 95, 99, or 100%. The plant performance may be increased by at least 5%. The plant performance may be increased by at least 10%. The plant performance may be increased by at least 30%. The plant performance may be increased by at least 50%. Plant performance is measured in a number of ways in various embodiments herein. For example, performance is measured as yield, nutritional value, flavonoid production, anthocyanin production, resistance to an insect challenge, resistance to a bacterial or fungal challenge, resistance to an abiotic stress such as drought, heat, cold, or nutrient stress, enhanced root growth or root architecture. Alternately, or in combination, plant performance is identified as reduction in herbicide, pesticide, or fertilizer application. Alternate definitions of plant performance are consistent with the disclosure herein.

Accordingly, transgenic plants expressing UV-B responsive genes results in growing crops such that pesticide use, herbicide use, fertilizer administration, or water administration is reduced relative to plants grown from non-transgenic seeds without any concomitant decrease in yield. In some cases, transgenic plants expressing UV-B responsive genes enable a substantial decrease in overall environmental impact without decrease in crop yield.

In some instances, plant performance is measured by at least one of a reduction in fertilizer, herbicide, insecticide, and pesticide use without affecting crop yield. Reduction to fertilizer, herbicide, insecticide, or pesticide use may be determined by comparison to the industry use for a crop over ten years, to the state-wide average, or the national average. The reduction of fertilizer, use may be at least 5%. In some cases, the reduction of fertilizer is in the range of about 5%-100%, 10%-90%, 20%-80%, 30%-70%, 40%-60%, 50%-95%, 65%-85%, or 75%-95%. In some instances, the reduction of herbicide use is at least 5%. In some cases, the reduction of herbicide is in the range of about 5%-100%, 10%-90%, 20%-80%, 30%-70%, 40%-60%, 50%-95%, 65%-85%, or 75%-95%. In some instances, the reduction of insecticide use is at least 5%. In some cases, the reduction of insecticide is in the range of about 5%-100%, 10%-90%, 20%-80%, 30%-70%, 40%-60%, 50%-95%, 65%-85%, or 75%-95%. In some instances, the reduction of pesticide use is at least 5%. In some cases, the reduction of pesticide is in the range of about 5%-100%, 10%-90%, 20%-80%, 30%-70%, 40%-60%, 50%-95%, 65%-85%, or 75%-95%.

In some instances, plant performance comprises administration of no more than at least one a standard fertilizer regimen, a standard pesticide regimen, a standard herbicide regimen, and a standard insecticide regimen to a transgenic plant comprising a UV-B responsive gene and improvements in crop yield. The term “standard regimen” refers to the industry standard. Crop yield may be increased by at least or about 5%, 10%, 15%, 20%, 25%, 30%, 35%, 40%, 45%, 50%, 55%, 60%, 65%, 70%, 75%, 80%, 85%, 90%, 95%, or more than 95% in a transgenic plant comprising a UV-B responsive gene.

Improvements in at least one of hardiness and plant performance may be determined from transgenic seeds, seedlings, or crops of seeds expressing a UV-B responsive gene that is responsive to methods described herein. For example, seedlings or seeds expressing a UV-B responsive gene are compared to seedlings or seeds that do not express a UV-B responsive gene. In some instances, improvements in the transgenic crops are compared to a crop grown under similar conditions but from seeds that do not express a UV-B responsive gene. Similar conditions may be similar environment or similar growing conditions. Environmental factors include, but are not limited to, sun exposure, temperature, soil composition, soil moisture, wind, humidity, and soil pH. Growing conditions, include but are not limited to, amount of watering, amount of pesticide, amount of herbicide, amount of insecticide, duration of priming, duration of germination, and timing of sowing. In some instances, the resultant crops are compared to crops grown at a same time. For example, the crops grown at the same time are grown on an adjacent or nearby field, or in a field, fileds or region reasonably expected to provide comparable growing conditions. In some instances, the resultant crops are compared to crops from a previous growing season. In some instances, a yield of the resultant crops is compared to a comparable crop or a number of comparable crops. In some cases yield is compared to a regional average or a historical average for a region or location. Yield may comprise improvements in at least one of plant performance and hardiness. In some instances, yield from a comparable crop is referred to standard yield. In some instances, the comparable crop is a crop that is grown at a same time or subject to similar growing conditions.

A variety of plants are suitable for improving plant performance using the systems and methods disclosed herein. Non-limiting examples of plant include lettuce, beans, broccoli, cabbage, carrot, cauliflower, cucumber, melon, onion, peas, peppers, pumpkin, spinach, kale, squash, sweetcorn, corn, maize, tomato, watermelon, alfalfa, canola, cotton, sorghum, soybeans, sugar beets, wheat, rice, grass, and commercially flowering plants such as tulips or roses.

In various embodiments, identity of a gene or polynucleotide associated with a phenotype is verified by sequencing, e.g., de novo sequencing, massive parallel sequencing, or next generation sequencing. For example, a gene or polynucleotide is verified by sequencing of nucleic acids from a transgenic plant sample.

The next-generation sequencing platform can be a commercially available platform including but is not limited to platforms for sequencing-by-synthesis, ion semiconductor sequencing, pyrosequencing, reversible dye terminator sequencing, sequencing by ligation, single-molecule sequencing, sequencing by hybridization, and nanopore sequencing. Platforms for sequencing by synthesis are available from, e.g., Illumina, 454 Life Sciences, Helicos Biosciences, and Qiagen. Illumina platforms can include, e.g., Illumina's Solexa platform, Illumina's Genome Analyzer, and are described in Gudmundsson et al (Nat. Genet. 2009 41:1122-6), Out et al (Hum. Mutat. 2009 30:1703-12) and Turner (Nat. Methods 2009 6:315-6), U.S. Patent Application Pub Nos. US20080160580 and US20080286795, U.S. Pat. Nos. 6,306,597, 7,115,400, and 7,232,656. 454 Life Science platforms include, e.g., the GS Flex and GS Junior, and are described in U.S. Pat. No. 7,323,305. Platforms from Helicos Biosciences include the True Single Molecule Sequencing platform. Platforms for ion semiconductor sequencing include, e.g., the Ion Torrent Personal Genome Machine (PGM) and are described in U.S. Pat. No. 7,948,015. Platforms for pyrosequencing include the GS Flex 454 system and are described in U.S. Pat. Nos. 7,211,390; 7,244,559; 7,264,929. Platforms and methods for sequencing by ligation include, e.g., the SOLiD sequencing platform and are described in U.S. Pat. No. 5,750,341. Platforms for single-molecule sequencing include the SMRT system from Pacific Bioscience and the Helicos True Single Molecule Sequencing platform.

### UV-B Treatment

Physical treatments on seeds have been primarily used to disinfect seeds from plant pathogens and insects. Exemplary physical treatments on seeds include application of hot water, hot air UV-C, X-rays, gamma rays, and electron beam irradiations. Yet, these treatments do not improve a seed or plant's systemic stress resilience, or overall plant performance over time. Details of such physical treatments are described in U.S. Pat. No. 8,001,722, which is incorporated hereby in its entirety.

The present disclosure provides systems and methods for treating seeds or plant material to improve overall plant performance, and plant resistance to biotic or abiotic stresses. Plant material may comprise a seed, a seedling, or a plant. Seeds, seed endosperms, seed coats or seed embryos can be treated with UV-B in a setup where the light source is controlled. In general, the setup is a room where sources of visible light and UV light are available. In some cases, the setup is indoors. In some cases, the setup is outdoors. In some cases, the setup is conducted at night. In some cases, the setup is conducted in daytime.

### Priming the Seeds

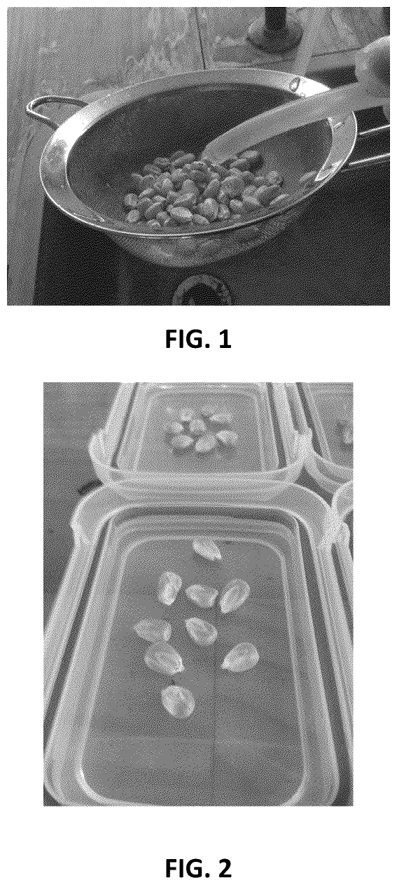

In representative examples, seeds or seed embryos are stored in a Tupperware container in a seed fridge or comparable environment. The number of seeds may be preselected. The number of seeds can be at most 1, 10, 20, 50, 100, 150, 200, 500, 1,000, or 10,000. The number of seeds can be at least 1, 10, 20, 50, 100, 150, 200, 500, 1,000, 10,000, or more. The number of seeds can be between 1-10000, 5-20, 30-50, 10-1000, 20-500, 50-100, or 500 to 2000. The required number of seeds can be washed under cold water (FIG. 1) or otherwise stratified prior to treatment. In some cases, washing the seed removes a red fungicide coating that may be present on the seeds. The seeds may be dried with a paper towel, a fabric, or a cloth.

The washed and dried seed may be arranged with the seed embryo-side up. In some instances, the washed and dried seed may be arranged with the seed embryo-side up into seed dishes. The embryo-side may be arranged to face toward the light source. The embryo-side may be arranged to face away from the light source for certain plant species such as maize or other monocot species. The seeds may be split across as many dishes as possible so as to reduce pseudo-replication (FIG. 2). In some cases, the seeds are arranged on trays in order to maximize or increase the efficacy of UV-B irradiation.

Consistent therewith, trays are disclosed having grooves such that a population of seeds distributed in the tray are oriented so as to maximize the efficacy of UV-B irradiation. In some cases, the tray grooves direct the seeds such that, for example, upon gentle administration of agitation to the tray, the seeds fall into an orientation such that they are positioned to maximize or increase UV-B administration efficacy. In various embodiments, trays are variously configured to accommodate seeds from a diversity of plant crops, such as maize, lettuce, rice, sorghum, cotton, alfalfa, wheat, or any other crop or ornamental seed plant disclosed herein.

The seeds may be kept at a temperature of about 0° C. to about 4° C., or other suitable temperature, for over 1, 2, 3, 4, 5, 6, 7, 8, 9, 10, 12, 15, 20, 24, or more than 24 hours. In some cases the seeds are kept at a temperature of about 0° C. to about 4° C. for over 24 hours. The seeds may be kept at a temperature in a range between −10-0, 0-4, 5-10, 15-40, 18-25, 20-22, or 24-28° C. The seeds may be kept at room temperature. The seeds may be kept at said temperature for over 1, 2, 3, 4, 5, 6, 7, 8, 9, 10, 12, 15, 20, 24, or more hours. The seeds may be kept at room temperature for over 24 hours.

Provided herein are methods and devices relating to UV-B administration, wherein UV-B is administered following a seed priming process or during a seed priming process. In some instances, UV-B is administered during such as concurrently with the seed priming process. In some instances, the seed priming process comprises methods for improving subsequent seed germination. In some instances, priming is at least one of hydropriming, osmopriming, redox priming, chemical priming, and hormonal priming. In some instances, priming comprises methods for affecting the osmotic potential or water potential of a seed environment. In some instances, methods affecting osmotic potential or water potential comprise a priming medium. In some instances, the priming medium is water. In some instances, the water is distilled water. In some instances, priming comprises a chemical that affects osmotic potential. For example, polyethylene glycol is used as a priming medium. Non-limiting examples of priming media include, but are not limited to, glycerol, mannitol, saline, and water. In some instances, the seed priming process includes treatment with an osmoticum, which helps to manage the seed hydration process.

In some instances, seeds are primed while being fully submerged. Seeds are often primed or stratified in seed trays with a water level that is maintained at about 1-2 mm above fully submerged seeds. In some cases, any floating seeds are tapped down until fully submerged. In some cases, water is refilled when water spills out or when water evaporates. The water level is monitored periodically. The water level is maintained at the top and water loss due to evaporation or spill is regularly refilled. For example, to imbibe the seeds, first the seed dishes are filled with 50 mL water. The water level is typically about 1-2 mm above fully submerged seeds. In some cases, tweezers may be used to tap down any floating seeds until all are fully submerged. The seeds can be arranged directly below the panels. In some cases, a cover may be placed over the seeds to avoid evaporation. The cover may be removed at start of treatment.

Priming duration may vary. In some instances, priming duration is at least or about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, 50 hours, 51 hours, 52 hours, 53 hours, 54 hours, 55 hours, 56 hours, 57 hours, 58 hours, 59 hours, 60 hours, or more than 60 hours. In some instances, priming duration is in a range of about 8 hours to about 44 hours. In some instances, priming duration is about 8 hours. In some instances, priming duration is about 18 hours. In some instances, priming duration is about 19.5 hours. In some instances, priming duration is about 20 hours. In some instances, priming duration is about 24 hours. In some instances, priming duration is about 27 hours. In some instances, priming duration is about 44 hours.

In some cases, the seeds are primed in at least one of the dark, light, and visible light.

In some embodiments, the priming process is operated by a control system connected to software, internet, or an electronic device, e.g., a computer. The control system may comprise a Raspberry Pie controller with wifi and zigbee module attached (FIG. 4).

Often the seeds are primed in a plant growth chamber at a suitable temperature. In some cases, the seeds are primed at about 25° C. In some instances, the seeds are primed at about 22° C. In some instances, the seeds are primed at about 10° C. The seeds may be primed at least at or about 10° C., 12° C., 15° C., 18° C., 20° C., 22° C., 25° C., 27° C., 30° C., 35° C., 40° C., 50° C., or more than 50° C. The seeds may be primed at most 10° C., 12° C., 15° C., 18° C., 20° C., 22° C., 25° C., 27° C., 30° C., 35° C., 40° C., or 50° C. The seeds may be primed at a temperature range of about 10° C.-50° C., 15° C.-30° C., 18° C.-25° C., or 20° C.-30° C.

The seeds may be primed at a relative humidity in a range between 30-100, 40-95, 50-90, 60-85, 65-75, 70-80, or 45-55%. The seeds may be primed at a relative humidity of at least or about 10%, 20%, 30%, 40%, 50%, 60%, 70%, 80%, 90%, 95%, 99%, or greater. Alternately, the seeds may be primed at a relative humidity of at most 10%, 20%, 30%, 40%, 50%, 60%, 70%, 80%, 90%, 95%, 99%, or 100%. The seeds may be primed at a relative humidity ranged from about 10%-100%, 15%-90%, 20%-80%, 30%-70%, 40%-60, 45%-75%, 50%-60%, 70%-90%, 85%-95%, or 95%-99%.

In some cases, priming the seed is conducted in a plant growth chamber where the ambient is adjusted to a suitable temperature, humidity and light brightness or intensity. The seeds may be primed at least at 10° C., 12° C., 15° C., 18° C., 20° C., 22° C., 25° C., 27° C., 30° C., 35° C., 40° C., 50° C., or more. The seeds may be primed at most 10° C., 12° C., 15° C., 18° C., 20° C., 22° C., 25° C., 27° C., 30° C., 35° C., 40° C., or 50° C. The seeds may be primed at a temperature ranged from 10° C.-50° C., 15° C.-30° C., 18° C.-25° C., or 20° C.-30° C. The seeds may be primed at 25° C. The seeds may be primed at a relative humidity of at least 10%, 20%, 30%, 40%, 50%, 60%, 70%, 80%, 90%, 95%, 99%, or more. The seeds may be primed at a relative humidity of at most 10%, 20%, 30%, 40%, 50%, 60%, 70%, 80%, 90%, 95%, 99%, or 100%. The seeds may be primed at a relative humidity ranged from 10%-100%, 15%-90%, 20%-80%, 30%-70%, 40%-60, 45%-75%, 50%-60%, 70%-90%, 85%-95%, or 95%-99%. The seeds may be primed at a relative humidity of 95%. The seeds may be primed in dark. The seeds may be primed in light. The seeds may be primed under visible light.

Provided herein are methods and devices relating to administration of UV-B, wherein light is administered using a light source. The light source may administer light of various wavelengths. For example, the light source is configured to emit one or more wavelengths of light in a range of about 300 nm and about 800 nm. In some instances, the light source emits one or more wavelengths in a range of about 280 nm to about 320 nm. In some instances, one or more light sources are used to emit the one or more wavelengths of light. The light source may be selected from the group consisting of a light emitting diode (LED), a laser, an incandescent light bulb, and a gas discharge bulb.

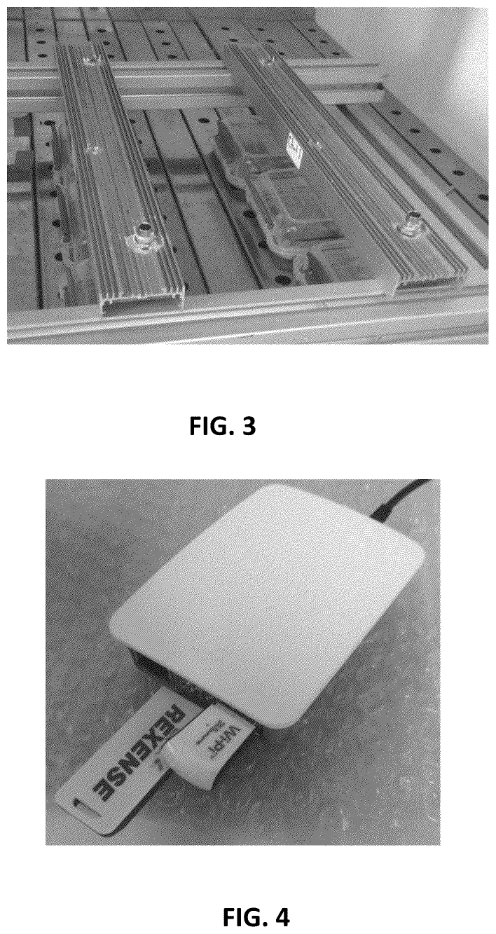

In some instances, the seeds are arranged in rows and placed under LED panels (FIG. 3). In some cases, the seeds are placed directly under the LED panel. A LED panel may be arranged above a row of seeds at about 20 mm, 40 mm, 60 mm, 80 mm, 100 mm, 120 mm, 150 mm, or 200 mm height. The LED panel may be arranged at a range of about 20 mm-200 mm, 40 mm-150 mm, 60 mm-120 mm, or 80 mm-100 mm. Often the distance between UV panels is about 10 mm. In some cases, the distance between UV panels is about 1 mm, 2 mm, 3 mm, 4 mm, 5 mm, 6 mm, 7 mm, 8 mm, 9 mm, 10 mm, 12 mm, 15 mm, or 20 mm. Alternately, the distance between UV panels is in a range of about 1 mm-20 mm, 2 mm-15 mm, 3 mm-10 mm, or 4 mm-9 mm. Often the minimum distance between UV and control panels is about 400 mm. In some instances, the minimum distance between the UV and central panels is about 50 mm, 100 mm, 150 mm, 200 mm, 250 mm, 300 mm, 350 mm, 400 mm, 450 mm, 500 mm, 600 mm, 700 mm, or 800 mm. Alternately, the distance between the UV and central panels is in a range about 50 mm-800 mm, 100 mm-700 mm, 150 mm-600 mm, 200 mm-500 mm, or 250 mm-400 mm.

In some cases, the seed trays are placed directly below LED panels at a height of about 8 cm or within a range of 7 cm, 6 cm, 5 cm, 4 cm, 3 cm, 2 cm, and 1 cm and at about or at least 20 cm between each treatment in order to prevent direct irradiance from adjacent treatments and covered before start of the treatment. In some cases, a distance between each treatment is in a range of about 20 cm-200 cm, 30 cm-100 cm, or 40 cm-90 cm. Often evaporated water is replaced, and the lid is removed prior to light treatment. Various LED configurations are consistent with the disclosure herein, and as is known to one of skill in the art, light intensity and distance from seeds can be varied in concert such that the total, mean or average dosage of UV-B light remains constant.

To prevent direct irradiance from adjacent treatments, the arrays may have a desired height and the distance between treatments may be controlled. In some cases, the arrays are at a height of about 1, 2, 3, 4, 5, 6, 7, 8, 9, 10, 12, 15, or 20 cm. The arrays can be about 8 cm. The distance between treatments can be at least 10, 15, 20, 30, 40, or 50 cm. The distance between treatments may be about 20 cm.

Often LED lights are configured to administer a peak irradiance wavelength of light, for instance at about 280 nm, a range within 10 nm, 9 nm, 8 nm, 7 nm, 6 nm, 5 nm, 4 nm, 3 nm, 2 nm, or 1 nm of 280 nm, or exactly 280 nm, at about 286 nm, a range within 10 nm, 9 nm, 8 nm, 7 nm, 6 nm, 5 nm, 4 nm, 3 nm, 2 nm, or 1 nm of 286 nm, or exactly 286 nm. Alternately, LED lights are configured to administer light at a standard white light spectrum which is supplemented by light in the UV-B range, for example at about 280 nm, a range within 10 nm, 9 nm, 8 nm, 7 nm, 6 nm, 5 nm, 4 nm, 3 nm, 2 nm, or 1 nm of 280 nm, or exactly 280 nm, at about 286 nm, a range within 10 nm, 9 nm, 8 nm, 7 nm, 6 nm, 5 nm, 4 nm, 3 nm, 2 nm, or 1 nm of 286 nm, or exactly 286 nm.

The UV can have photon energy in a range between 3-15, 3.5-10, 5-12, 3.8-4.8, or 4-4.5 eV. The UV can have photon energy about 3.8-4.5 eV. The UV can have photon energy about 3.94-4.43 eV. The UV can have photon energy in a range between 0.2-2.2, 0.3-2.0, 0.4-1.8, 0.5-1.5, 0.6-1.0, or 0.8-1.2 aJ. The UV can have photon energy about 0.5-0.8 aJ. The UV can have photon energy about 0.631-0.71 aJ.

Various irradiances of UV-B may be used. In some cases, the irradiance is in a range of about 4×10⁻⁵ W cm⁻² s⁻¹ to about 1.3×10⁻⁴ W cm⁻² s⁻¹. The irradiance range can be at about 4×10⁻⁵ W cm⁻² s⁻¹, exactly 4×10⁻⁵ W cm⁻² s⁻¹, or at least 4×10⁻⁵ W cm⁻² s⁻¹. In some cases, the irradiance is in a range of about 1.3×10⁻⁴ W cm⁻² s⁻¹, exactly 1.3×10⁻⁴ W cm⁻² s⁻¹, or more than 1.3×10⁻⁴ W cm⁻² s⁻¹. The irradiance range can be about 4×10⁻⁵ W cm⁻² s⁻¹-6×10⁻⁵ W cm⁻² s⁻¹, 6×10⁻⁵ W cm⁻² s⁻¹-8×10⁻⁵ W cm⁻² s⁻¹, 8×10⁻⁵ W cm⁻² s⁻¹-1×10⁻⁴ W cm⁻² s⁻¹, or 1×10⁻⁴ W cm⁻² s⁻¹-1.5×10⁻⁵ W cm⁻² s⁻¹. The UV can have irradiance in a range between 0.5×10⁻⁵ to 5.0×10⁻⁴, 1.0×10⁻⁵ to 3.0×10⁻⁴, 2.0×10⁻⁵ to 3.0×10⁻⁴, 2.5×10⁻⁵ to 2.0×10⁻⁴, or 3.0×10⁻⁵ to 1.5×10⁻⁴ W cm⁻² s⁻¹. The UV can have irradiance in a range between 4×10⁻⁵ to 1.3×10⁻⁴ W cm⁻² s⁻¹. Dosage may change in relation to treatment protocols such as hydration protocols.

Various dosages of UV-B are contemplated herein. In some instances, the dosage is in the range of about 0.01 kJ m⁻² to about 368 kJ m⁻². In some instances, the dosage is about 0.01 kJ m²-368 kJ m², 0.1 kJ m⁻²-300 kJ m⁻², 1 kJ m⁻²-250 kJ m⁻², 10 kJ m⁻²-200 kJ m⁻², 100 kJ m⁻²-150 kJ m², 200 kJ m⁻²-300 kJ m⁻², 250 kJ m⁻²-350 kJ m⁻², or 300 kJ m⁻²-368 kJ m⁻². In some instances, the dosage is in the range of about 0.1 to about 12 kJ m⁻². In some instances, the dosage is about 13 kJ m⁻². The light treatment may be at a dose of about 13 kJ m⁻², exactly 13 kJ m⁻², or at least 13 kJ m⁻². In some instances, the dosage is about 37 kJ m⁻². In some instances, the dosage is about 69 kJ m⁻². In some instances, the dosage is about 78 kJ m⁻². In some instances, the dosage is about 98 kJ m⁻². In some instances, the dosage is about 100 kJ m⁻². The light treatment may be at a dose of about 100 kJ m⁻², exactly 100 kJ m⁻², or more than 100 kJ m⁻². In some instances, the dosage is about 125 kJ m⁻². In some instances, the dosage is about 204 kJ m⁻². The light treatment may be at a dose range of about 13 kJ m⁻² to 100 kJ m⁻². The UV-B can be at a dose in a range of about 1 kJ m⁻²-1000 kJ m⁻², 10 kJ m⁻²-800 kJ m⁻², 20 kJ m⁻²-600 kJ m², 30 kJ m⁻²-400 kJ m⁻², 50 kJ m⁻²-200 kJ m⁻², 100 kJ m⁻²-150 kJ m⁻², 30 kJ m⁻²-60 kJ m⁻², or 150 kJ m⁻²-250 kJ m⁻². In some instances, the UV-B is in a range of 0.1 kJ m⁻²-20 kJ m⁻², 20 kJ m⁻²-40 kJ m⁻², 40 kJ m⁻²-60 kJ m², 60 kJ m⁻²-80 kJ m⁻², or 80 kJ m⁻²-100 kJ m⁻². The UV-B can be at a dose of about 0.01-368, 0.1-300, 1-250, 10-200, 100-150, 200-300, 250-350, or 300-368 kJ m².

The seeds may be treated with UV for a sufficient time to elicit a biological effect. The seeds may be treated with UV for about 0.1-100, 1-80, 5-60, 10-40, 20-30, 2-18, or 5-10 h. The seeds may be treated with UV for about 1, 2, 3, 4, 5, 6, 7, 8, 9, 10, 12, 14, 16, 18, 20, 30, 40, 50, 100 or more h. The seeds may be treated with UV for about 9 h. The seeds may be treated with UV for about 21 h or within less than 30 minutes of 21 hours. The seeds may be treated with UV for about 30 h. The seeds may be treated with UV-B in a range between 280-310 nm. The seeds may be treated with UV-B at about 280 nm. In some cases, the primed seeds are treated with visible light, e.g. blue and/or red light, and UV-B simultaneously. In some cases, the primed seeds are treated with visible light, e.g. blue and/or red light, and UV-B sequentially in any order. In some cases, the primed seeds are treated with visible light, e.g. blue and/or red light, without UV-B exposure. Seedlings treated with visible light only can serve as controls for the effect of UV-B treatment. In some cases, the seeds treated with UV-B and/or visible light are not primed. The visible light can have a photon number in a range between 10-550, 20-500, 40-450, 45-400, 50-350, 100-300, or 100-200 μmol. The visible light can have a photon number about 20 μmol. The visible light can have a photon number about 50 μmol. The visible light can have a photon number about 400 μmol.

Following light treatment, the seeds are often dried using a paper towel to remove excess water and then air dried for 72 hours. The seeds may be dried with a paper towel then left to air dry for less than, about, exactly, or at least 12 hours, 24 hours, 36 hours, 48 hours, 60 hours, 72 hours, 84 hours, 96 hours, or more hours. In some cases, the seeds are subsequently stored and covered. The dried seeds may be uncovered and stored in a container.

UV-B treatment may be initiated at different time-points or durations. For instance, UV-B treatment is variously applied to at least one of prior to seed hydration, prior to seed germination, during initial germination (e.g. following moisture application for seed germination), and during a priming treatment. In some instances, UV-B is administered during seed priming.

### Sowing the Seeds

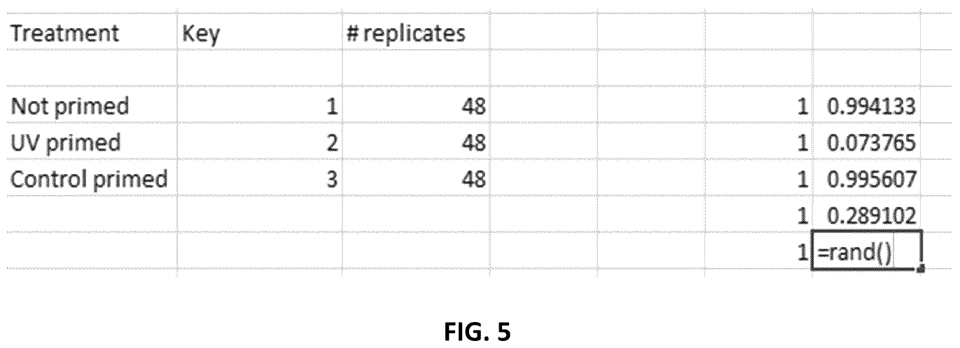

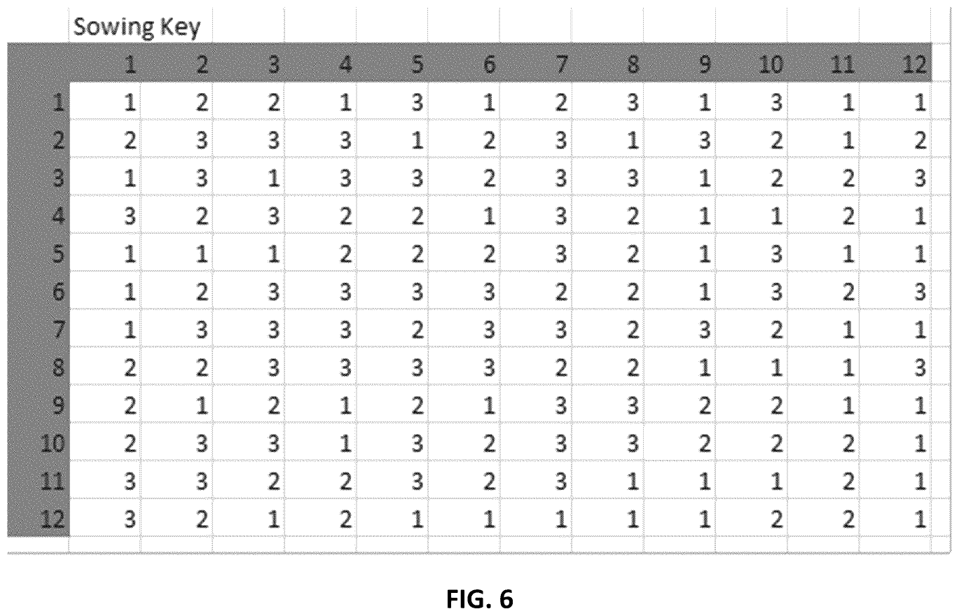

After priming and UV treatment (e.g. UV-B at a range between 280-320 nm, for example), the seeds may be sowed systematically or randomly. The procedures for sowing the seeds may involve preparing a randomized sowing key in excel. The number of replicates per treatment may be determined and a list of all replicates may be created (FIG. 5). For example, the random number of every adjacent cell is assigned by using=rand( ) in excel. The replicates may be sorted by the random numbers, which may shuffle the list of replicates. The shuffled list of replicates may be used to create a randomized 12×12 sowing key (FIG. 6). The seeds may be sowed in a 12×12 tray accordingly to the 12×12 sowing key.

### Dualex

Once the seeds germinate, dualex may be performed to assess flavonol, anthocyanin and chlorophyll contents in the leaves. Dualex allows for performing real-time and non-destructive measurements. The assessment of polyphenolic compounds in leaves is based on the absorbance of the leaf epidermis through the screening effect it procures to chlorophyll fluorescence. Typically, the indices calculated by Dualex are: (i) Anth, for the anthocyanin index; (ii) Chl, for the chlorophyll index; (iii) Flav, for the flavonols index; and (iv) NBI, for the nitrogen balance index.

Dualex may be completed as soon as the cotyledons are big enough, for example, on day 5 after priming the seeds.

### Harvest

Seedlings may be harvested for analysis. Typically, seedlings are harvested by 21 days old or by stage V2. Fresh shoot, leaf, and root may be collected and their fresh weights may be measured. In addition, dried weights of the collected shoot, leaf and root may be measured for further analysis.

The seedlings may be inspected for enhancement in at least one physiological condition comprising increased biomass in at least one of flavonoid levels, anthocyanin levels, size, dry weight, nitrogen index, shoot dry weight, shoot fresh weight, shoot length, radical length, pigment production, leaf size, hypocotyl length, chlorophyll level, leaf area, and root dry weight. The seedlings may be inspected for improved resilience following at least one of heat, flood, drought, frost, unusual climate events, salinity stress, and high visible light stress.

The physiological condition in the seedlings may be increased by a significant percentage when compared to the counterpart plants that do not express a UV-B responsive gene that are responsive to the UV-B regimen disclosed herein. The physiological condition may be increased by about 5-100, 10-90, 20-80, 30-70, 40-60, 50-95, 65-85, or 75-95%. The physiological condition may be increased by at least 5, 10, 15, 20, 25, 30, 35, 40, 45, 50, 55, 60, 65, 70, 76, 80, 85, 90, 95, 99, or 100%. The physiological condition may be increased by at least 5%. The physiological condition may be increased by at least 10%. The physiological condition may be increased by at least 30%. The physiological condition may be increased by at least 50%.

The seedlings may grow into immature or young plants and subsequently mature plants. The mature plants may exhibit enhanced growth or plant performance. For growth performance or plant performance, mature plants may be inspected for enhancement in various aspects including at least one of as improvements in at least one of flavonoid levels, anthocyanin levels, size, dry weight, nitrogen index, shoot dry weight, shoot fresh weight, shoot length, radical length, pigment production, leaf size, hypocotyl length, chlorophyll level, leaf area, root dry weight, fruit, flower, height, leaf surface area, hormone index, nitrogen index, hardiness, root growth, root architecture, and seed germination rate. In some cases, mature plants are inspected for improved quality comprising at least one of a longer shelf-life, a resistance to bruising or post-harvesting handling, an increased nutrient value, an improved taste, an improved shape, an improved color, an improved size, and an improved texture. Mature plants may be measured during or following sowing.

In some embodiments, UV-B treatment is applied to immature plants and the immature plants are left to grow to maturity. The overall plant performance of the plant may be monitored. For example, the matured plants are subjected to at least one of a biotic stress test and an abiotic stress test. Mature plants are inspected for diseases in plants caused by pathogens (infectious organisms) and environmental conditions (physiological factors). Mature plants may be inspected for infections caused by organisms include fungi, oomycetes, bacteria, viruses, viroids, virus-like organisms, phytoplasmas, protozoa, nematodes and parasitic plants. Mature plants may be inspected for leaf disease, ear rot disease, stalk rot disease, and seeding and root disease. Mature plants may be inspected for drought or salinity resistance.

For biotic stress test, mature plants may be inspected for leaf disease, ear rot disease, stalk rot disease, seeding and root disease, or combinations thereof.

For leaf disease, mature plants may be inspected for symptoms including, but not limited to, anthracnose leaf blight, common rust, common smut, crazy top, eyespot, Goss's bacterial wilt and blight, gray leaf spot, Holcus spot, maize chlorotic dwarf virus infection, maize dwarf mosaic virus infection, northern corn leaf blight, Stewart's wilt, northern leaf spot, physoderma brown spot, sorghum downy mildew, southern rust, and southern corn leaf blight. The mature plant may be inspected for at least one symptom of leaf disease.

For ear rot disease, mature plants are inspected for symptoms including, but not limited to, Aspergillus ear rot, Diplodia ear rot, Fusarium kernel or ear rot, Gibberella or red ear rot, Nigrospora ear rot or cob rot, and Penicillium ear rot. The mature plant may be inspected for at least one symptom of ear rot disease.

For stalk rot disease, mature plants may be inspected for symptoms including, but not limited to, Anthracnose stalk rot, bacterial stalk rot, charcoal stalk rot, Diplodia stalk rot, Fusarium stalk rot, Gibberella stalk rot, Pythium stalk rot, and red root rot. The mature plant may be inspected for at least one symptom of stalk rot disease.

For seedling and root disease, mature plants may be inspected for symptoms including, but not limited to, Stewart's wilt and corn nematodes. The mature plant may be inspected for at least one symptom of seedling and root disease.

For abiotic stress test, mature plants may be inspected for resistance to at least one of flood, drought, frost, unusual climate events, salinity stress, and high visible light stress.

For growth performance, mature plants are inspected for enhancement in various aspects including fruit, flower, height, leaf surface area, hormone index, nitrogen index, hardiness, and seed germination rate.

The growth performance may be indicated by enhancement in at least one physiological condition. The enhanced physiological condition may comprise fruit size, fruit taste, pollen, root, leaf, stem, flower, and biomass in dry weight or fresh weight. The enhanced physiological condition may comprise increased production or secretion of flavonoid, anthocyanin, ascorbate acid, or tocopherol. The enhanced physiological condition may comprise plant hormones, auxins, gibberellins, cytokinins, and brassinosteroids. The enhanced physiological condition may comprise increased ovule, carpel, embryo, pericarp, or endosperm. The enhanced physiological condition can be increased production or secretion of flavonoid. The enhanced physiological condition can be increased production or secretion of anthocyanin. The enhanced physiological condition can be increased production or secretion of ascorbate acid. The enhanced physiological condition can be increased production or secretion of tocopherol. The enhanced physiological condition can be increased production or secretion of a plant ripening hormone. The enhanced physiological condition can be increased production or secretion of a seed germinating hormone.

The physiological condition in the mature plants may be increased by a significant percentage when compared to the counterpart plants that do not express a UV-B responsive gene that is responsive to the UV-B regimen disclosed herein. The physiological condition may be increased by about 5-100, 10-90, 20-80, 30-70, 40-60, 50-95, 65-85, or 75-95%. The physiological condition may be increased by at least 5, 10, 15, 20, 25, 30, 35, 40, 45, 50, 55, 60, 65, 70, 76, 80, 85, 90, 95, 99, or 100%. The physiological condition may be increased by at least 5%. The physiological condition may be increased by at least 10%. The physiological condition may be increased by at least 30%. The physiological condition may be increased by at least 50%.

### Transgenic Plants

In some aspects, systems and methods disclosed herein comprise generating transgenic plants that express a UV-B responsive gene identified as described herein. A transgenic plant may comprise at least one of the identified UV-B responsive genes. In some cases, a transgenic plant is generated to express more than one of the identified UV-B responsive genes. Generation of transgenic plant may comprise (i) irradiating a seed or a seedling of a plant with a defined photon energy, wavelength, dose and duration of UV-B in a range of from 280-320 nm, (ii) identifying a phenotype of interest of a mature plant of the UV-B irradiated seedling, (iii) isolating a polynucleotide that is associated with the phenotype of interest, (iv) validating a function of the isolated polynucleotide and correlating the isolated gene's function with the phenotype of interest, and transforming the isolated gene into a wildtype plant to recover a transgenic plant that demonstrates the UV-B irradiation phenotype in the absence of UV-B treatment. In some instances, the UV-B responsive gene is identified in at least one of a seed, seedling, and mature plant. In some instances, the UV-B responsive gene is identified in a seed. Following identification of the UV-B responsive gene, the UV-B responsive gene may be transformed into a plant cell. The plant cell may be at least one of a seed cell, a seedling cell, and a mature plant cell. In some instances, the plant cell is a seed cell. The UV-B responsive gene may be expressed in a plant cell such that the UV-B responsive gene is expressed in at least one of a seed, seedling, and mature plant.

Transformation in some cases comprises cloning a nucleic acid segment of interest and replicating it in a plasmid in a bacterium. The plasmid may further comprise a selection marker, e.g., an antibody that is resistance to a specific condition. Seeds of the plants may be sown on soil or agarose that contains a specific antibiotic and only the seeds that have the polynucleotide of interest encoding resistance to this particular antibiotic will grow. The offspring is verified for homologous for the insertion and grown to maturity. Generation of transgenic plant may involve detecting homologous sequences of polynucleotide of interest, and generating recombinant constructs that comprise the gene of interest. It is understood that a polynucleotide of interest may be a full length polynucleotide or partial thereof that encodes a gene. The recombinant constructs can be introduced and integrated into the genome of the plant to generate stable transgenic lines. Detailed description for generating transplant is described in U.S. Pat. Nos. 5,159,135; 5,744,693; 8,898,818, which are incorporated by reference in their entireties.

In one aspect, disclosed herein are systems and methods for generating a stable transgenic plant that produces a desirable agronomic trait or a phenotype of interest such as enhanced crop yield, growth rate, hardiness, stress resistance, and pathological resistance that is observed upon treatment of a wild-type seed with UV-B radiation. The method comprises exposing a seed or seedling of a plant to a defined photon energy, wavelength, dose and duration of UV-B in a range of from 280-320 nm to induce the desirable agronomic trait. The plant can be maize, kale, cabbage, or any edible plants. The seed or seedling may be exposed to a light source with enriched UV-B with photon energy in a range between 4×10⁻⁵ to 1.3×10⁻⁴ W cm⁻² s⁻¹ or in a range between 3.94-4.43 eV. The seed or seedlings may be exposed to a light source with enriched UV-B with a wavelength in a range between 280-320 nm, 285-315 nm, or 290-310 nm. The seed or seedlings may be exposed to a light source with enriched UV-B with a wavelength at about 280 nm. The seed or seedlings may be exposed to a light source with enriched UV-B with a wavelength at about 286 nm. The seed or seedlings may be exposed to a light source with enriched UV-B. The UV-B can be at a dose of about 0.01-368, 0.1-300, 1-250, 10-200, 100-150, 200-300, 250-350, or 300-368 kJ m⁻². The seed or seedlings may be exposed to a light source with enriched UV-B at dose of 13 kJ m⁻². The seed or seedlings may be exposed to a light source with enriched UV-B at dose of 100 kJ m⁻². In some instances, the seed is inspected for a desirable agronomic trait. In some instances, the seedling is inspected for a desirable agronomic trait. The seed or seedling is allowed to growth into a mature plant that may produce flowers, fruits, and seeds. The mature plant is allowed to grow without supplementary enriched UV-B irradiation. In some cases, the mature plant may be exposed to the supplementary enriched UV-B irradiation. The mature plant is inspected for a desirable agronomic trait, e.g., enhanced crop yield. For example, the mature plant produces increased dry mass when compared to a sibling plant that has not been irradiated with enriched UV-B during seed or seedling stage. The improvement may be evaluated by comparing, e.g., dry mass, of the mature plant and its sibling plant. The desirable agronomic trait may be enhanced by 10%, 20, 30%, 40%, 50%, 60%, 70%, 80%, 90%, 95%, 99%, or more. In some cases, the desirable agronomic trait is enhanced by at least 10%. In some cases, the desirable agronomic trait is enhanced by at least 30%. In some cases, the desirable agronomic trait is enhanced by at least 50%.

Once a desirable agronomic trait or a phenotype of interest is identified, nucleic acids may be extracted and isolated from the mature plant. In some instances, the nucleic acids are extracted from plant material. Plant material may comprise at least one of a seed, a seedling, and a mature plant. The nucleic acids can be genomic DNA. The nucleic acids can be RNA (e.g., total RNA, mRNA). In some cases, the RNA is reverse transcribed to complementary DNA (cDNA). The sequence of the isolated nucleic acids may be determined by sequencing using the techniques and platforms described herein or standard techniques or platforms in the field. In some embodiments, the methods further comprise determining the function of the isolated nucleic acids by manipulating the expression level of the isolated nucleic acid, or a downstream responder of the isolated nucleic. In some cases, manipulating the isolated nucleic acids may directly affect the phenotype of interest. The isolated nucleic acid may for example encode a transcription factor that activates or suppresses expression of a downstream responder gene, which determines the phenotype of interest or which operates in a signaling cascade that leads to a phenotype of interest. In some cases, manipulating the isolated nucleic acids may indirectly affect the phenotype of interest. The isolated nucleic acid may encode, for example, a ligand of a receptor, which upon binding the ligand and receptor pair, activates a signaling pathway and triggers a cascade of responding genes. In some cases, the isolated nucleic acids have previously been determined to play a role in producing the phenotype of interest. In some case, the role of the isolated nucleic acids in producing the phenotype of interest is novel. A variety of techniques may be applied to verify the function of the isolated nucleic acids. In one example, verification may be achieved by gene knockdown, genome editing, CRISPRs, TALENs, RNAi or gene knockout of the isolated nucleic acids, wherein the expression level of the isolated nucleic acids is reduced or abolished. Without being bound to any theory, the offspring plant will not produce the phenotype of interest due to the absence or reduction of the isolated nucleic acids. In another example, verification may be achieved by overexpression an exogenous copy of the isolated nucleic acids in a plant. Without being bound to any theory, the offspring plant will produce increased number of enhances the phenotype of interest due to the presence of the excess copy of isolated nucleic acids.

The isolated and verified nucleic acids may be introduced into a vector or plasmid for delivery to a plant. In some instances, the isolated and verified nucleic acids are introduced into a vector or plasmid for delivery to a plant cell. In some instances, the plant cell is at least one of seed cell, a seedling cell, and a mature plant cell. In some instances, the plant cell is a seed cell. The vector or plasmid comprising the isolated and verified nucleic acids may be introduced in to bacteria, yeast, delivery vehicles, or the like. In some cases, the vector or plasmid comprising the isolated and verified nucleic acids is introduced into bacteria via transformation. The bacteria are allowed to grow to a large population of bacteria containing the vector or plasmid. The method further comprises contacting the bacteria with a plant and allowing the bacteria to insert the isolated and verified nucleic acids into the plant cells. The vector or plasmid may further comprise a selection marker, e.g., an antibiotic resistance gene, that allows a plant with the inserted vector or plasmid to grow in the presence of the particular antibiotic. The plant is allowed to grow and produce seeds, which germinate and grow into progeny plants. The seeds or seedlings may be subjected to supplementary UV-B irradiation prior to subsequent growth phase using systems and methods described herein. Alternately, the progeny may exhibit a phenotype that mimics UV-B irradiation in the absence of such treatment. The method may further comprise verifying insertion of the nucleic acids in the parent plants. The verification may comprise isolating nucleic acid from the parent plant and sequencing the isolated nucleic acid for the presence of sequencing encoding the vector, plasmid or the antibiotic resistant gene, or combinations thereof. The nucleic acids may be isolated from the parent plant seeds, seedlings, or from the mature parent plants. Parent plants which have the inserted nucleic acids are allowed to grow and produce seeds in the absence of the supplementary enriched UV-B irradiation. The methods further comprise inspecting the parent plants for the phenotype of interest.

Seeds of the parent plants may be subjected to supplementary UV-B irradiation prior to subsequent growth phase using systems and methods described herein and allowed to grow into mature offspring plants. The offspring plants may be grown in the absence of the supplementary enriched UV-B irradiation and inspected for the phenotype of interest. Offspring plants which produce the phenotype of interest are kept and breed to generate stable transgenic lines.

In some embodiments, the methods further comprise identifying a homologous sequence of the isolated nucleic acids that governs the phenotype of interest. Homologous sequence found in a maize plant may be used to identify homologous sequence in a kale plant. A variety of methods may be used to identify homologous sequences in different organisms. As an example, the isolated nucleic acids from a maize is used as a query sequence to blast against a database of nucleic acids from a collection of species or a species of interest. The highest ranked hit sequence may be used to back-blast for the query as a way to verify the accuracy of the query blasting result. Numerous search engines may be used for nucleic acid blast, including search engines described herein or widely used in the field. Non-limiting example of blast search engines are NCBI BLAST (blast.ncbi.nlm.nih.gov/Blast.cgi), UCSC Genome Browser (genome.ucsc.edu/) and UniProt (www.uniprot.org/).

In some embodiments, the methods further comprise generating transgenic lines of a second plant species comprising a homologous nucleic acid sequence of an isolated nucleic acid that is used to generate a transgenic line of a first plant species, wherein the transgenic line of the first plant species and the second plant species produce a similar phenotype of interest. Generation of transgenic lines in different plant species can be achieved by using systems and methods described herein.

### Methods for Detecting Homologous Sequences

Homologous sequences (homologs) identified herein may also be used to change the phenotype of plants so as to recapitulate phenotypes observed from transgenic or otherwise mutant plants of different species. Genes may be identified in one species, such as a model organism or a first crop species, and used to identify homologous genes in a second species, such as an agriculturally relevant species. In some instances, genes are identified in at least one of a seed, a seedling, and a mature plant. In some instances, genes are identified in a seed. The homologues of the second species are used to recapitulate the first species' phenotype in the second species. Often, the second species is an agriculturally important plant species, including but not limited to, crops such as soybean, wheat, corn, maize, sweetcorn, potato, cotton, rice, oilseed rape (including canola), sunflower, alfalfa, sugarcane and turf; or fruits and vegetables, such as banana, blackberry, blueberry, strawberry, and raspberry, cantaloupe, carrot, cauliflower, coffee, cucumber, eggplant, grape, honeydew, lettuce, mango, melon, onion, papaya, peas, peppers, pineapple, spinach, squash, tobacco, tomato, watermelon, rosaceous fruits (such as apple, peach, pear, cherry and plum) and vegetable brassicas (such as broccoli, cabbage, cauliflower, Brussel sprouts and kohlrabi). Other crops, fruits and vegetables whose seed's phenotype may be changed include barley, currant, avocado, citrus fruits such as oranges, lemons, grapefruit and tangerines, artichoke, cherries, nuts such as the walnut and peanut, endive, leek, roots, such as arrowroot, beet, cassava, turnip, radish, yam, sweet potato, beans, and cannabis.

Genes that are homologs of the identified polypeptide sequences will typically share at least 40% amino acid sequence identity. More closely related homologues may share at least 50%, 60%, 65%, 70%, 75% or 80% sequence identity with the disclosed sequences. Factors that are most closely related to the disclosed sequences share at least 85%, 90% or 95% sequence identity. At the nucleotide level, the sequences will typically share at least 40% nucleotide sequence identity, at least 50%, 60%, 70%, 80%, 85%, 90%, 95% or 97% sequence identity.

Homologs from the same plant, different plant species or other organisms may be identified using database sequence search tools, such as the Basic Local Alignment Search Tool (BLAST) (Altschul et al. (1990) Basic local alignment search tool. J. Mol. Biol. 215:403-410; and Altschul et al. (1997) Gapped BLAST and PSI-BLAST: A New Generation of Protein Database Search Programs Nucleic Acid Res. 25: 3389-3402). Several sequence analysis programs (blastp, blastn, blastx, tblastp, tblastn and tblastx) are available from several sources, including GCG (Madison, Wis.) and the National Center for Biotechnology Information (NCBI, Bethesda, Md.). When using the sequence analysis program tblastn, the BLOSUM-62 scoring matrix (Henikoff, S. and Henikoff, J. G. (1992) Proc. Natl. Acad. Sci. USA 89: 10915-10919) may be employed. Sequences with the highest scores and an exemplary cutoff E value threshold for tblastn less than −70, and can be less than −100, are identified as homologous sequences.

Generally, a sequence from a first species is used to query a sequence database of a second species. The strongest hit, or group of strongest hits, are recovered from the search, and are used to query a sequence dataset of the first species. If the original query sequence is retuned (or a set of closely related sequences are returned), then the sequences from the first species and second species are likely to represent homologous sequences.

Substitutions, deletions and insertions introduced into a gene of interest are also envisioned by this disclosure. Amino acid substitutions can be of single residues. Amino acid substitutions can be of multiple residues. Insertions can be on the order of about from 1 to 10, 1 to 100, 1 to 1,000, or 1 to 10,000 amino acid residues, or more. Deletions may range about from 1 to 10, 1 to 100, 1 to 1,000, or 1 to 10,000 residues, or more. Deletions or insertions may be made in adjacent pairs, e.g. a deletion of two residues or insertion of two residues. Substitutions, deletions, insertions or any combination thereof may be combined to arrive at a final construct. The mutations that are made in the DNA encoding the protein may not place the sequence out of reading frame and may not create complementary regions that could produce secondary mRNA structure.

Substitutions are those in which at least one residue in the amino acid sequence has been removed and a different residue inserted in its place. Such substitutions generally are made in accordance with the following Table 1 when it is desired to finely modulate the characteristics of the protein. Table 1 shows amino acids which may be substituted for an amino acid in a protein and which are typically regarded as conservative substitutions.

TABLE 1
Amino acids which may be substituted for an amino acid in a protein
which are typically regarded as conservative substitutions

| Residue | Conservative Substitutions |
|---|---|
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | leu, Val |
| Leu | Ile; Val |
| Lys | Arg; Gln; Glu |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

Table 1 presents a representative list of conservative substitutions suitable for some proteins. Other lists of conservative substitutions, in some cases listing substantially more or different sets of conservative residues, are known in the art. Substitutions that are less conservative than those in Table 1 may be selected by selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in protein properties will be those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine.

Homologous sequences also encompass polypeptide sequences that are modified by chemical or enzymatic means. Modifications include acetylation, carboxylation, phosphorylation, glycosylation, modified amino acids and the like. Protein modification techniques are illustrated in Ausubel et al. (eds) Current Protocols in Molecular Biology, John Wiley & Sons (1998).

The degeneracy of the genetic code further widens the scope of the present invention as it enables major variations in the nucleotide sequence of a DNA molecule while maintaining the amino acid sequence of the encoded protein. Overall, UVR8-COP1-HY5 UV-B pathway modulators that are homologs of the disclosed sequences may often share at least 30% nucleotide sequence identity with a homologous sequence. More closely sequences may share at least 50%, 60%, 65%, 70%, 75% or 80% sequence identity with the disclosed nucleotide sequences. UVR8-COP1-HY5 UV-B pathway modulators that are most closely related to the disclosed nucleotide sequences share at least 85%, 90% or 95% sequence identity with one or more of the disclosed corn UVR8-COP1-HY5 UV-B pathway modulators proteins.

Homologs of the corn UVR8-COP1-HY5 UV-B pathway modulators may alternatively be obtained by immuno-screening an expression library. With the provision herein of the disclosed UVR8-COP1-HY5 UV-B pathway modulators nucleic acid sequences, the polypeptide may be expressed and purified in a heterologous expression system (e.g., E. coli) and used to raise antibodies (monoclonal or polyclonal) specific for the UVR8-COP1-HY5 UV-B pathway modulators. Antibodies may also be raised against synthetic peptides derived from UVR8-COP1-HY5 UV-B pathway modulators amino acid sequences. Methods of raising antibodies are well known in the art and are described in Harlow and Lane (1988) Antibodies: A Laboratory Manual, Cold Spring Harbor Laboratory, New York. Such antibodies can then be used to screen an expression library produced from the plant from which it is desired to clone the UVR8-COP1-HY5 UV-B pathway modulators DNA homolog, using the methods described above. The selected cDNAs can be confirmed by sequencing and enzymatic activity.

### Recombinant Constructs

Any of the identified sequences may be incorporated in a recombinant construct for expression in plants. A number of recombinant vectors suitable for stable transformation of plant cells or for the establishment of transgenic plants have been described including those described in Weissbach and Weissbach, (1989) Methods for Plant Molecular Biology, Academic Press, and Gelvin et al., (1990) Plant Molecular Biology Manual, Kluwer Academic Publishers. Specific examples include those derived from a Ti plasmid of Agrobacterium tumefaciens, as well as those disclosed by Herrera-Estrella, L., et al., Nature 303: 209 (1983), Bevan, M., Nucl. Acids Res. 12: 8711-8721 (1984), Klee, H. J., Bio/Technology 3: 637-642 (1985) for dicotyledonous plants.

Non-Ti vectors can be used to transfer the DNA into monocotyledonous plants and plant cells by using free DNA delivery techniques. Such methods may involve, for example, the use of liposomes, electroporation, microprojectile bombardment, silicon carbide wiskers, viruses and pollen. By using these methods transgenic plants such as wheat, rice (Christou, P., Bio/Technology 9: 957-962 (1991)) and corn (Gordon-Kamrn, W., Plant Cell 2: 603-618 (1990)) are produced. An immature embryo can also be a good target tissue for monocots for direct DNA delivery techniques by using the particle gun (Weeks, T. et al., Plant Physiol. 102: 1077-1084 (1993); Vasil, V., Bio/Technology 10: 667-674 (1993); Wan, Y. and Lemeaux, P., Plant Physiol. 104: 37-48 (1994), and for Agrobacterium-mediated DNA transfer (Hiei et al., Plant J. 6: 271-282 (1994); Rashid et al., Plant Cell Rep. 15: 727-730 (1996); Dong, J., et al., Mol. Breeding 2: 267-276 (1996); Aldemita, R. and Hodges, T., Planta 199: 612-617 (1996); Ishida et al., Nature Biotech. 14: 745-750 (1996)).

Typically, plant transformation vectors include one or more cloned plant genes (or cDNAs) under the transcriptional control of 5′ and 3′ regulatory sequences and a dominant selectable marker. The one or more plant genes may encode for the same protein or more than one protein that performs different functions. Such plant transformation vectors typically also contain a promoter (e.g., a regulatory region controlling inducible or constitutive, environmentally-or developmentally-regulated, or cell- or tissue-specific expression), a transcription initiation start site, a ribosome binding site, an RNA processing signal, a transcription termination site, and/or a polyadenylation signal.

Examples of constitutive plant promoters which may be useful for expressing the UVR8-COP1-HY5 UV-B pathway modulator sequence include: the cauliflower mosaic virus (CaMV) 35S promoter, which confers constitutive, high-level expression in most plant tissues (see, e.g., Odel et al., (1985) Nature 313:810); the nopaline synthase promoter (An et al., (1988) Plant Physiol. 88:547); and the octopine synthase promoter (Fromm et al., (1989) Plant Cell 1: 977).

A variety of plant gene promoters that regulate gene expression in response to environmental, hormonal, chemical, developmental signals, and tissue also can be used for expression of the UVR8-COP1-HY5 UV-B pathway modulator sequence in plants, as illustrated seed-specific promoters (such as the napin, phaseolin or DC3 promoter described in U.S. Pat. No. 5,773,697), fruit-specific promoters that are active during fruit ripening (such as the dru 1 promoter (U.S. Pat. No. 5,783,393), or the 2A11 promoter (U.S. Pat. No. 4,943,674) and the tomato polygalacturonase promoter (Bird et al. (1988) Plant Mol. Biol. 11:651), pollen-active promoters such as PTA29, PTA26 and PTA13 (U.S. Pat. No. 5,792,929), promoters active in vascular tissue (Ringli and Keller (1998) Plant Mol. Biol. 37:977-988), flower-specific (Kaiser et al, (1995) Plant Mol. Biol. 28:231-243), pollen (Baerson et al. (1994) Plant Mol. Biol. 26:1947-1959), carpels (Ohl et al. (1990) Plant Cell 2:837-848), pollen and ovules (Baerson et al. (1993) Plant Mol. Biol. 22:255-267), auxin-inducible promoters (such as that described in van der Kop et al (1999) Plant Mol. Biol. 39:979-990 or Baumann et al. (1999) Plant Cell 11:323-334), cytokinin-inducible promoter (Guevara-Garcia (1998) Plant Mol. Biol. 38:743-753), promoters responsive to gibberellin (Shi et al. (1998) Plant Mol. Biol. 38:1053-1060, Willmott et al. (1998) 38:817-825) and the like.

Plant transformation vectors may also include RNA processing signals, for example, introns, which may be positioned upstream or downstream of the open reading frame sequence. In addition, the expression vectors may also include additional regulatory sequences from the 3′-untranslated region of plant genes, e.g., a 3′ terminator region to increase mRNA stability of the mRNA, such as the PI-II terminator region of potato or the octopine or nopaline synthase 3′ terminator regions.

Plant transformation vectors may also include dominant selectable marker genes to allow for the ready selection of transformants. Such genes may include those encoding antibiotic resistance genes (e.g., resistance to hygromycin, kanamycin, bleomycin, G418, streptomycin or spectinomycin) and herbicide resistance genes (e.g., phosphinothricin acetyltransferase).

An increase of UVR8-COP1-HY5 UV-B pathway activity in a transgenic plant to obtain improved growth may be obtained by introducing into plants antisense constructs based on the UVR8-COP1-HY5 UV-B pathway modulator cDNA. For antisense suppression, the UVR8-COP1-HY5 UV-B pathway modulator cDNA is arranged in reverse orientation relative to the promoter sequence in the transformation vector. The introduced sequence need not be the full length UVR8-COP1-HY5 UV-B pathway modulator cDNA or gene, and need not be exactly homologous to the UVR8-COP1-HY5 UV-B pathway modulator cDNA or gene found in the plant type to be transformed. Generally, however, where the introduced sequence is of shorter length, a higher degree of homology to the native UVR8-COP1-HY5 UV-B pathway modulator sequence will be needed for effective antisense suppression. The introduced antisense sequence in the vector may be 5, 10, 15, 20, 25, 30, 35, 40, 45, 50, or more nucleotides in length, and improved antisense suppression may be observed as the length of the antisense sequence increases. The length of the antisense sequence in the vector may be greater than 10, 20, 30, 40, 50, 60, 70, 80, 90, 100, 120, 150, 180, 200, 250, 300, 400, 500, or more nucleotides. Transcription of an antisense construct as described results in the production of RNA molecules that are the reverse complement of mRNA molecules transcribed from the endogenous UVR8-COP1-HY5 UV-B pathway modulator gene in the plant cell. Suppression of endogenous UVR8-COP1-HY5 UV-B pathway modulator gene expression can also be achieved using ribozymes. Ribozymes are synthetic RNA molecules that possess highly specific endoribonuclease activity. The production and use of ribozymes are disclosed in U.S. Pat. No. 4,987,071 to Cech and U.S. Pat. No. 5,543,508 to Haselhoff. The inclusion of ribozyme sequences within antisense RNAs may be used to confer RNA cleaving activity on the antisense RNA, such that endogenous mRNA molecules that bind to the antisense RNA are cleaved, which in turn leads to an enhanced antisense inhibition of endogenous gene expression.

Constructs in which RNA encoding the UVR8-COP1-HY5 UV-B pathway modulator cDNA (or homologs thereof) is over-expressed may also be used to obtain co-suppression of the endogenous UVR8-COP1-HY5 UV-B pathway modulator gene in the manner described in U.S. Pat. No. 5,231,020 to Jorgensen. Such co-suppression (also termed sense suppression) does not require that the entire UVR8-COP1-HY5 UV-B pathway modulator cDNA be introduced into the plant cells, nor does it require that the introduced sequence be exactly identical to the endogenous UVR8-COP1-HY5 UV-B pathway modulator gene. However, as with antisense suppression, the suppressive efficiency will be enhanced as (1) the introduced sequence is lengthened and (2) the sequence similarity between the introduced sequence and the endogenous UVR8-COP1-HY5 UV-B pathway modulator gene is increased.

Constructs expressing an untranslatable form of the UVR8-COP1-HY5 UV-B pathway modulator gene may also be used to suppress the expression of endogenous UVR8-COP1-HY5 UV-B pathway activity. Methods for producing such constructs are described in U.S. Pat. No. 5,583,021 to Dougherty et al. Such constructs are made by introducing a premature stop codon into the UVR8-COP1-HY5 UV-B pathway modulator gene.

Modulation of the UVR8-COP1-HY5 UV-B pathway, or another UV-B signaling pathway, may also be modulated using less blunt approaches, such as point mutations or other mutations that affect dimerization of UVR8, for example, such that signaling component activity is affected independent of overall protein accumulation levels. UVR8, for example, forms COP1 heterodimers that effect signaling, and stabilizing UVR8 homodimers affects their ability and frequency of dimerization with COP1. Similarly, increasing expression of a negative regulator of signaling, may also impact the plant phenotype so as to recapitulate UV-B supplementation. RUP1 and 2, for example are negative regulators of UV-B signaling, and affecting their expression levels may modulate this pathway.

Transgenic Plants with Modified UVR8-COP1-HY5 UV-B Pathway Modulator Expression

Once a construct comprising a nucleotide sequence encoding a UVR8-COP1-HY5 UV-B pathway modulator gene of the present disclosure has been isolated, standard techniques may be used to express the cDNA in plants in order to modify that particular seed characteristic. In some instances, the construct is expressed in a plant cell. In some instances, the plant cell is at least one of a seed cell, a seedling cell, and a mature plant cell. In some instances, following expression of the construct in the plant cell, at least one of a seed, seedling, and mature plant expresses the construct. In many embodiments herein, the plant cell is a cell of a plant seed.

Exemplary plants to be transformed may be any higher plant, including monocotyledonous and dicotyledenous plants. Suitable protocols are available for Leguminosae (alfalfa, soybean, clover, etc.), Umbelliferae (carrot, celery, parsnip), Cruciferae (cabbage, radish, rapeseed, broccoli, etc.), Curcurbitaceae (melons and cucumber), Gramineae (wheat, corn, rice, barley, millet, etc.), Solanaceae (potato, tomato, tobacco, peppers, etc.), and various other crops. See protocols described in Ammirato et al. (1984) Handbook of Plant Cell Culture—Crop Species. Macmillan Publ. Co. Shimnamoto et al. (1989) Nature 338:274-276; Fromm et al. (1990) Bio Technology 8:833-839; and Vasil et al. (1990) Bio/Technology 8:429-434.

Transformation and regeneration of both monocotyledonous and dicotyledonous plant cells and the selection of the most appropriate transformation technique will be determined by the practitioner. The plant cells may be at least one of a seed cell, a seedling cell, and a mature plant cell. The choice of method will vary with the type of plant to be transformed; those skilled in the art will recognize the suitability of particular methods for given plant types. Suitable methods may include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium tumefaciens (AT) mediated transformation.

Successful examples of the modification of plant characteristics by transformation with cloned cDNA sequences which serve to illustrate the current knowledge in this field of technology, and which are herein incorporated by reference in their entireties, include: U.S. Pat. Nos. 5,571,706; 5,677,175; 5,510,471; 5,750,386; 5,597,945; 5,589,615; 5,750,871; 5,268,526; 5,780,708; 5,538,880; 5,773,269; 5,736,369 and 5,610,042.

Following transformation, plants are selected using a dominant selectable marker incorporated into the transformation vector. Typically, such a marker will confer antibiotic or herbicide resistance on the transformed plants, and selection of transformants can be accomplished by exposing the plants to appropriate concentrations of the antibiotic or herbicide.

After transformed plants are selected and grown to maturity, they can be assayed using the methods described herein to determine whether UVR8-COP1-HY5 UV-B pathway activity has been altered as a result of the introduced recombinant polynucleotide, such as by analyzing mRNA expression using Northern blots, microarrays, q-PCR, or by visual inspection of plant seed or biochemical assays.

After establishing that the transformed plants do overexpress the UVR8-COP1-HY5 UV-B pathway modulator gene, the plants may be used to isolate an endogenous plant growth chemical that affects fruit and seed size, and yields in plants. The large fruits, stems, leaves or flowers of the transformed plants are harvested and the chemicals present in them fractionated by standard fractionation into organic phases and water-soluble fractions. These fractions may be assayed for bioactivity on immature plants in culture. The active fractions that improve plant growth are further purified and sufficient material is obtained to identify the structure of the hormonally produced chemical in the transformed plants. Identified chemicals may be useful for spraying on fruit, vegetable and grain crops to increase fruit, vegetable and grain sizes and yields.

Additionally, plants or plant material expressing the UVR8-COP1-HY5 UV-B pathway modulator gene may be employed for screening other compounds that may control parthenocarpy or fruit, stem, leaf or flower size in a plant. The method entails first introducing a compound into the plant or a host cell. The compound may be introduced by topical administration of the exogenous compound and then monitoring the effect of the exogenous compound on the expression of the UVR8-COP1-HY5 UV-B pathway modulator polypeptide or the expression of the polynucleotide encoding the same so as to detect changes in expression. Changes in the expression of the UVR8-COP1-HY5 UV-B pathway modulator polypeptide may be monitored by use of polyclonal or monoclonal antibodies, two-dimensional polyacrylamide electrophoresis (2D-PAGE), Clustered Regularly Interspersed Short Palindromic Repeats (CRISPR), genome engineering or the like. Changes in the expression of the corresponding polynucleotide sequence may be detected by use of microarrays, Northern blots, q-PCR, or any other technique for monitoring changes in mRNA expression. Exemplary techniques are exemplified in Ausubel et al. (eds) Current Protocols in Molecular Biology, John Wiley & Sons (1998).

In some embodiments, transgenic plants are generated using genome engineering by introducing targeted double-strand breaks (DSBs) in a DNA sequence to be modified. The DNA sequence is the polynucleotide, or a partial thereof, that is identified to respond to UV-B treatment described herein. Detailed description of generating transgenic plant with DSB genome engineering is described in U.S. Application No: 20140273235, which is incorporated hereby in its entirety.

Genome engineered DSBs activate cellular DNA repair pathways, which can be harnessed to achieve desired DNA sequence modifications near the break site. Targeted DSBs can be introduced using sequence-specific nucleases (SSNs), a specialized class of proteins that includes transcription activator-liked (TAL) effector endonucleases, zinc-finger nucleases (ZFNs), and homing endonucleases (HEs). Recognition of a specific DNA sequence may be achieved through an interaction with specific amino acids encoded by the SSNs. Prior to the development of TAL effector endonucleases, a challenge of engineering SSNs was the unpredictable context dependencies between amino acids that bind to DNA sequence. While TAL effector endonucleases greatly alleviated this difficulty, their large size (on average, each TAL effector endonuclease monomer contains 2.5-3 kb of coding sequence) and repetitive nature may hinder their use in applications where vector size and stability is a concern (Voytas, Annu Rev Plant Biol, 64, 130301143929006, 2012).

The CRISPR-associated (CRISPR/Cas) system includes a recently identified type of SSN. CRISPR/Cas molecules are components of a prokaryotic adaptive immune system that is functionally analogous to eukaryotic RNA interference, using RNA base pairing to direct DNA or RNA cleavage. Directing DNA DSBs requires two components: the Cas9 protein, which functions as an endonuclease, and CRISPR RNA (crRNA) and tracer RNA (tracrRNA) sequences that aid in directing the Cas9/RNA complex to target DNA sequence (Makarova et al., Nat Rev Microbiol, 9(6):467-477, 2011). The modification of a single targeting RNA can be sufficient to alter the nucleotide target of a Cas protein. In some cases, crRNA and tracrRNA can be engineered as a single cr/tracrRNA hybrid to direct Cas9 cleavage activity (Jinek et al., Science, 337(6096):816-821, 2012). The CRISPR/Cas system can be used in bacteria, yeast, humans, and zebrafish, as described elsewhere (see, e.g., Jiang et al., Nat Biotechnol, 31(3):233-239, 2013; Dicarlo et al., Nucleic Acids Res, doi:10.1093/nar/gkt135, 2013; Cong et al., Science, 339(6121):819-823, 2013; Mali et al., Science, 339(6121):823-826, 2013; Cho et al., Nat Biotechnol, 31(3):230-232, 2013; and Hwang et al., Nat Biotechnol, 31(3):227-229, 2013). The utility of the CRISPR/Cas system in plants has not previously been demonstrated.

CRISPR/Cas systems can be used for plant genome engineering. In some instances, CRISPR/Cas systems are used to generate transgenic plants. Cas9, when expressed or transfected in cells alongside a gRNA, allows for the targeted introduction or deletion of genetic information via a complex with a Clustered Regularly Interspaced Short Palindromic Repeats (CRISPR) sequence of mRNA. The cells may be a plant cell, wherein the plant cell is at least one of a seed cell, a seedling cell, and a mature plant cell. Following expression in a plant cell, at least one of a seed, seedling, and mature plant expresses the gene. In a CRISPR/Cas9 process, a gRNA shepherds the Cas9 enzyme to a specific stretch of DNA. Cas9 then cleaves the DNA to disable or repair a gene. Cas9 can be delivered to a plant cell by a virus (e.g. DNA virus or RNA virus). CRISPR/Cas systems can be performed in plant leaf tissue by targeting DSBs to integrated reporter genes and endogenous loci. CRISPR/Cas systems can be adapted for use in protoplasts and whole plants, and in viral-based delivery systems. Finally, multiplex genome engineering can be demonstrated by targeting DSBs to multiple sites within the same genome.

In general, the CRISPR/Cas systems include at least two components: the RNAs (crRNA and tracrRNA, or a single cr/tracrRNA hybrid) targeted to a particular sequence in a plant cell (e.g., in a plant genome, or in an extrachromosomal plasmid, such as a reporter), and a Cas9 endonuclease that can cleave the plant DNA at the target sequence. In some cases, the CRISPR/Cas systems also include a nucleic acid containing a donor sequence targeted to a plant sequence. The endonuclease can be used to create targeted DNA double-strand breaks at the desired locus (or loci), and the plant cell can repair the double-strand break using the donor DNA sequence, thereby incorporating the modification stably into the plant genome.

The construct(s) containing the crRNA, tracrRNA, cr/tracrRNA hybrid, endonuclease coding sequence, and, where applicable, donor sequence, can be delivered to a plant cell using, for example, biolistic bombardment. Alternatively, the system components can be delivered using Agrobacterium-mediated transformation, insect vectors, grafting, or DNA abrasion, according to methods that are standard in the art, including those described herein. In some embodiments, the system components can be delivered in a viral vector (e.g., a vector from a DNA virus such as, without limitation, geminivirus, cabbage leaf curl virus, bean yellow dwarf virus, wheat dwarf virus, tomato leaf curl virus, maize streak virus, tobacco leaf curl virus, tomato golden mosaic virus, or Faba bean necrotic yellow virus, or a vector from an RNA virus such as, without limitation, a tobravirus (e.g., tobacco rattle virus, tobacco mosaic virus), potato virus X, or barley stripe mosaic virus.

After a plant or plant cell is infected or transfected with an endonuclease encoding sequence and a crRNA and a tracrRNA, or a cr/tracrRNA hybrid (and, in some cases, a donor sequence), any suitable method can be used to determine whether GT or targeted mutagenesis has occurred at the target site. In some embodiments, a phenotypic change can indicate that a donor sequence has been integrated into the target site. Such is the case for transgenic plants encoding a defective GUS:NPTII reporter gene, for example. PCR-based methods also can be used to ascertain whether a genomic target site contains targeted mutations or donor sequence, and/or whether precise recombination has occurred at the 5′ and 3′ ends of the donor.

In some embodiments, TILLING (Targeting Induced Local Lesions in Genomes) is used to generate transgenic plants. “TILLING” as used herein comprises introduction of mutations, even random mutations, and then screening for mutations, exhibited as mismatch complexes in heteroduplex DNA, in the mutagenized resultant DNA. Various embodiments of TILLING are described as comprising mutagenesis, for example using a chemical mutagen such as Ethyl methanesulfonate (EMS), ethidium bromide, or other point mutant, with a DNA screening-technique, such as a technique that identifies mutations such as insertions, deletions or single base mutations (also called point mutations) in a gene or locus of interest. Some embodiments of the method rely on the formation and detection of DNA heteroduplexes that result when multiple alleles are annealed, such as during or resulting from PCR, and are then melted and slowly cooled. A “bubble” forms at the mismatch position of two DNA strands differing at a single position. In some embodiments the bubble is then cleaved by a single stranded nuclease. The products are then separated by size, using any of a number of approaches. The presence of size variation represented by the cleavage products indicates the presence of allelic variation in the target locus. Mismatches may be due to induced mutation, heterozygosity within an individual, or natural variation between individuals.

Once a stable transgenic plant material is identified to produce the desired agronomic traits, the stable transgenic plant material is selected from the rest of the plants. The selected stable transgenic plant material may be used to breed with siblings who yield the same desired agronomic traits. The selected stable transgenic plant material may be used to breed with other stable transgenic plant material that yield a different agronomic trait, thereby creating mixed agronomic traits. Plant material may comprise at least one of seeds, seedlings, and mature plants.

ISOLATION of Nucleic Acid from a Plant

### Plant RNA Isolation

The general protocol for isolating plant RNA may include a variety of RNA containing materials, for example, from plant stems, leaves, roots, seeds and flowers. Detailed description of plant RNA isolation is described in U.S. Pat. No. 6,875,857, which is incorporated hereby in its entirety. Plant tissues can be treated with lysis buffer comprising a buffering component to provide a suitable chemical environment for extraction and recovery of RNA analysis. Plant tissue can be ground to a coarse or fine powder. In some instances, the plant tissue comprises at least one of a seed, a seedling, and a mature plant. When the plant material is a cell culture, the cells may be mixed, e.g., by rocking, with the extraction medium for about five minutes. When the plant material is tissue material, the powder may be mixed with the extraction medium for about 20 minutes. The plant material may be mixed with reagent until ground tissue is thoroughly suspended.

The extract preparation may be centrifuged to remove cellular debris. A step of filtration or straining can also be used. Concentrated NaCl may be added to the preparation, for example about 0.25 parts of 5 M NaCl. An organic extraction solvent, such as CHCl₃ may be added to the supernatant and mixed therewith. Aqueous and organic phases can be separated by centrifugation. The aqueous phase is subjected to alcohol, e.g., ethanol, precipitation to obtain isolated RNA.

There are a variety of commercially available kits, products, and reagents for isolating plant RNA. For example, RNA can be isolated by using NucleoSpin® RNA Plant (Takara, Cat. No: 740949.50), RNeasy Plant Mini Kit (Qiagen, Cat. No: 74903), PureLink® Plant RNA Reagent (Thermo Fisher Scientific, Cat. No: 12322012), MagMAX™-96 Total RNA Isolation Kit (Thermo Fisher Scientific, Cat. No: AM1830), PureLink® RNA Mini Kit (Thermo Fisher Scientific, Cat. No: 12183018A), mirVana™ miRNA Isolation Kit, with phenol (Thermo Fisher Scientific, Cat. No: AM1560), Spectrum™ Plant Total RNA Kit (Sigma-Aldrich, Cat. No: STRN10-1KT), Plant RNA Isolation Mini Kit (Agilent Technologies, Cat. No: 5188-2780), ZR Plant RNA MiniPrep™ (Zymo Research, Cat. No: R2024), MasterPure™ Plant RNA Purification Kit (Epicenter, Ca. No: MPR09100), or miRCURY™ RNA Isolation Kits—Cell & Plant (Exiqon, Cat. No: 300110).

In some cases, plant tissues can be extracted with acetone at −70° C. to remove polyphenolics. The pellet is then homogenized in the presence of 0.1% (v/v) TRITON® X-100 (octyl phenol polyethoxylate), 15 mM DTT (dithiothreitol) and phenol. The homogenization process releases RNA, DNA, and proteins. Proteins are removed by phase separation in an organic extraction phase. Then, DNA is removed by centrifugation on a cesium chloride cushion. The removed protein or DNA can be collected.

In another case, guanidinium isothiocyanate is used to disrupt the plant tissue, and RNA is then recovered by centrifugation on a cesium trifluoroacetate cushion. Other methods may use cationic or anionic detergents to release the nucleic acids followed by either multiple alcohol precipitation or phenol extraction, and lithium chloride precipitation to remove DNA from the isolated RNA.

In some embodiments, the RNA isolation reagents comprise two or more of the following components: one or more non-ionic detergent, one or more ionic detergent, one or more chelator, one or more reducing agent, one or more antibacterial agent (e.g., sodium azide, at about 0.5%).

The primary detergent may be any of the non-ionic detergents available, or in use. Non-limiting examples include IGEPAL® (tergitol) (tert-octylphenoxy poly(oxyethylene)ethanol) (NP-40 replacement), TRITON®s, (TRITON®X-100 (octyl phenol polyethoxylate)), TWEEN®20 (polyoxyethylene sorbitan monolaurate) and the like. The non-ionic detergent may be chosen for its ability to extract RNA without co-isolation of DNA. In some cases, non-ionic detergent is present at a concentration of about 0.1-4% by volume. In other cases, non-ionic detergent is present at a concentration of about 0.5-3%, or about 1%-2%. For instance, the non-ionic detergent is IGEPAL® (tergitol) (tert-octylphenoxy poly(oxyethylene) ethanol) at a concentration of 1% by volume.

The helper-detergent or secondary detergent may be any of the cationic or anionic detergents available (e.g. SDS, CTAB) and improves RNA yields especially at high reducing agent concentrations, for example, 2-mercaptoethanol concentrations of about 40%. The concentration of ionic detergent may be about 0.01%-0.5%, or about 0.01-0.1%. For instance, ionic detergent is SDS at a concentration of about 0.02% or up to about 0.2%, depending on the plant material and the concentrations of other components, especially the reducing agent.

The detergents may be selected in an amount so as to render the cell membranes permeable so that agents can enter the cell cytoplasmic domain and RNA can exit the cell cytoplasmic domain. The amounts of the detergents and reducing agent(s) may be selected to retain degradative components within the cell so that, for example, harmful enzymes are removed with the cellular debris.

In some cases, the greater the concentration of 2-mercaptoethanol or similar reducing agent in the formulation, the higher the concentration of secondary (ionic) detergent is included. Higher concentration of 2-mercaptoethanol higher quality of isolated RNA.

The chelator may also provide the ‘salt’ requirement to maintain the cell membrane and/or the cell nucleus at physiological salt conditions, to avoid osmotic disruption. Chelator may be chosen from those commonly in use. For example, EDTAs, EGTAs, citrates (such as sodium citrate), citric acids, salicylic acids, salts of salicylic acids, phthalic acids, 2,4-pentanedines, histidines, histidinol dihydrochlorides, 8-hydroxyquinolines, 8-hydroxyquinoline, citrates and o-hydroxyquinones are representative of chelators known in the art. Alternatively, one component of the reagent may be used to provide the salt strength, NaCl, KCl, etc., and a different agent (e.g., betaine) may be used as the chelator. The chelator may present at a concentration of about 0.01-0.25 M. The chelator may present at a concentration of about 0.01 M, 0.02 M, 0.03 M, 0.04 M, 0.05 M, 0.06 M, 0.07 M, 0.08 M, 0.09 M, 0.1 M, 0.15 M, 0.2 M, 0.25 M, or more. The chelator can be EDTA at a concentration of about 0.1 M.

The reducing agent may be chosen from 2-mercaptoethanol or from any number that would replace 2-mercaptoethanol (e.g., DTT, or other mercaptans). The reducing agent may present at a concentration of about 1%-40% volume. The reducing agent is present at a concentration of about 10%-40%, 15%-20%, 20%-30%, or 20%-40%. The concentration of 2-Mercaptoethanol may be about 1%-40%. The 20% or 40% was found to produce RNA at good yield and high quality in selected tissues. For some applications, about 4% 2-mercaptoethanol is used.

The reagent may include an antibacterial agent, e.g., sodium azide, to extend the shelf life of the reagent. Accordingly, an antibacterial agent is not required when freshly prepared components are combined shortly before use. Also, any antibacterial agent that extends shelf life without unduly degrading the quality of the RNA obtained is therefore suitable for use in the present invention. The amount of antibacterial agent depends on the agent and the storage conditions and should be selected so as not to interfere with the extraction process and to provide the desired shelf life.

All components and surfaces that might contact the sample may be RNase free.

A subset of the components can be prepared in advance, separately, or in combination and be combined with the remaining components at a time before use or at the time of use to obtain the working formulation.

cDNA Synthesis

### Reverse Transcription

In some embodiments, cDNA is synthesized from mRNA through the process of reverse transcription. Reverse transcription can be performed directly on cell lysates (for example, a cell lysate prepared as described above), by adding a reaction mix for reverse transcription directly to the cell lysate. In alternative embodiments, the total RNA or mRNA can be purified after cell lysis, for example through the use of column based (e.g., Qiagen RNeasy Mini kit Cat. No. 74104, ZymoResearch Direct-zol RNA Cat. No. R2050) or magnetic bead purification (e.g., Agencourt RNAClean XP, Cat. No. A63987). Reverse transcription of mRNA to cDNA may be performed using well established methods. In some embodiments, the reverse transcription is combined with a template switching step to improve the yield of longer (e.g., full length) cDNA molecules. In certain embodiments, the reverse transcriptase used has tailing or terminal transferase activity, and synthesizes and anchors first-strand cDNA in one step. In certain embodiments, the reverse transcriptase is a Moloney Murine Leukemia Virus (MMLV) reverse transcriptase, for example, SMARTscribe™ (Clontech, Cat. No. 639536) reverse transcriptase, SuperScript II™ reverse transcriptase (Life Technologies, Cat. No. 18064-014), or Maxima H Minus™ reverse transcriptase. (Thermo Fisher Scientific, Cat. No. EP0753)

Generation of cDNA may comprise template switching. Template switching introduces an arbitrary sequence at the 3′ end of the cDNA that is designed to be the reverse complement to the 3′ end of a cDNA synthesis primer. In some embodiments, the synthesis of the first strand of the cDNA can be directed by a cDNA synthesis primer (CDS) that includes an RNA complementary sequence (RCS). In some embodiments, the RCS is at least partially complementary to one or more mRNA species in an individual mRNA sample, allowing the primer to hybridize to at least some mRNA species in a sample to direct cDNA synthesis using the mRNA as a template. The RCS can comprise oligo (dT) sequence that binds to many mRNA species, or it can be specific for a particular mRNA species, for example, by binding to an mRNA sequence of a gene of interest. Alternatively, the RCS can comprise a random sequence, such as random hexamers. To avoid the CDS self-priming, a non-self-complementary sequence can be used.

A template-switching oligonucleotide that includes a portion which is at least partially complementary to a portion of the 3′ end of the first strand of cDNA generated by the reverse transcription can also be used in the methods of the invention. Because the terminal transferase activity of reverse transcriptase typically causes the incorporation of two to five cytosines at the 3′ end of the first strand of cDNA synthesized, the first strand of cDNA can include a plurality of cytosines, or cytosine analogues that base pair with guanosine, at its 3′ end to which the template-switching oligonucleotide with a 3′ guanosine tract can anneal. During the template switching step, the template-switching oligonucleotide is extended to form a double stranded cDNA. Thus, in some embodiments, a template-switching oligonucleotide can include a 3′ portion comprising a plurality of guanosines or guanosine analogues that base pair with cytosine. Exemplary guanosines or guanosine analogues include, but are not limited to, deoxyriboguanosine, riboguanosine, locked nucleic acid-guanosine, and peptide nucleic acid-guanosine. The guanosines can be ribonucleosides or locked nucleic acid monomers. A locked nucleic acid is an RNA nucleotide wherein the ribose moiety has been modified with an extra bridge connecting the 2′ oxygen and the 4′ carbon. A peptide nucleic acid is an artificially synthesized polymer similar to DNA or RNA, wherein the backbone is composed of repeating N-(2-aminoethyl)-glycine units linked by peptide bonds.

In some embodiments, the reverse transcription and template switching comprise contacting an mRNA sample with two nucleic acid primers. In certain embodiments, the first nucleic acid primer (e.g., a template-switching oligonucleotide) comprising a 5′ poly-isonucleotidecytosine-isoguanosine-isocytosine sequence, an internal adapter sequence, and a 3′ guanosine tract. In certain embodiments, the 5′ poly-isonucleotide sequence comprises an isocytosine, or an isoguanosine, or both. In certain embodiments, the 5′ poly-isonucleotide sequence comprises an isocytosine-isoguanosine-isocytosine sequence. Incorporating non-natural nucleotides, such as an isocytosine or an isoguanosine into template-switching primers can reduce background and improve cDNA synthesis (Kapteyn et al., BMC Genomics. 11:413 (2010)). In some embodiments, the 3′ guanosine tract comprises two, three, four, five, six, seven, eight, nine, ten, or more guanosines. In certain embodiments, the 3′ guanosine tract comprises three guanosines. In some embodiments, the adapter sequence is 12 to 32 nucleotides in length, for example, 22 nucleotides in length.

In certain embodiments, the second nucleic acid primer (e.g., a cDNA synthesis primer) comprises a 5′ blocking group, an internal adapter sequence, a barcode sequence, a unique molecular identifier (UMI) sequence, a complementarity sequence, and a 3′ dinucleotide sequence comprising a first nucleotide and a second nucleotide, wherein the first nucleotide of the dinucleotide sequence is a nucleotide selected from adenine, guanine, and cytosine, and the second nucleotide of the dinucleotide sequence is a nucleotide selected from adenine, guanine, cytosine, and thymine. Optionally, to sequence bulk RNA or lysates, the bar code can be omitted from the cDNA synthesis primer and an extra 6 base pairs can be added to the UMI sequence. In particular embodiments, the 5′ blocking group is selected from biotin, an inverted nucleotide (e.g., inverted dideoxy-T), a fluorophore, an amino group, and iso-dG or isodC. In some embodiments, the internal adapter sequence is 23 to 43 nucleotides in length. In some embodiments, the barcode sequence is 4 to 20 nucleotides in length, for example, 4, 5, 6, 7, 8, 9, 10, 11, 12, 13, 14, 15, 16, 17, 18, 19, or 20 nucleotides in length. In some embodiments, the UMI sequence is 6 to 20 nucleotides in length, for example, 6, 7, 8, 9, 10, 11, 12, 13, 14, 15, 16, 17, 18, 19, or 20 nucleotides in length. In some embodiments, the complementarity sequence is a poly(T) sequence. In particular embodiments, the complementarity sequence is 20 to 40 nucleotides in length, for example, 20, 21, 22, 23, 24, 25, 26, 27, 28, 29, 30, 31, 32, 33, 34, 35, 36, 37, 38, 39, or 40 nucleotides in length.

The UMI sequences provide a robust guard against amplification biases. More particularly, each UMI is present only once in a population of second nucleic acid primers. Thus, each UMI is incorporated into a unique cDNA sequence generated from a cellular mRNA, and any subsequent amplification steps will not alter the one UMI to one mRNA ratio. In certain embodiments, the UMI sequence, rather than being 10 nucleotides in length, is 5, 6, 7, 8, 9, 11, 12, 13, 14, 15, 16, 17, 18, 19, 20, 21, 22, 23, 24, 25, 26, 27, 28, 29, 30, or more nucleotides in length. The length should be selected to provide sufficient unique sequences for the population of cells to be tested. In some cases, the lengths selected are at least two nucleotide differences between any pair of UMIs.

Barcode sequences enable each cDNA sample generated by the above method to have a distinct tag, or a distinct combination of tags, such that once the tagged cDNA samples have been pooled, the tag can be used to identify the single cell from which each cDNA sample originated. Thus, each cDNA sample can be linked to a single cell, even after the tagged cDNA samples have been pooled and amplified. In other words, the use of the foregoing nucleic acids permits deconvolution of pooled data to single cell/well resolution. This is particularly advantageous for facilitating the application of this technology to screening assays.

In some embodiments, a nucleic acid useful in the invention can contain a non-natural sugar moiety in the backbone, for example, sugar moieties with 2′ modifications such as addition of a halogen, alkyl-substituted alkyl, SH, SCH₃, OCN, Cl, Br, CN, CF₃, OCF₃, SO₂CH₃, OSO₂, NO₂, N₃, or NH₂. Similar modifications also can be made at other positions on the sugar. Nucleic acids, nucleoside analogs or nucleotide analogs having sugar modifications can be further modified to include a reversible blocking group, a peptide linked label, or both. In those embodiments comprising a 2′ modification, the base can have a peptide-linked label.

A nucleic acid can include native or non-native bases. In some embodiments, a native deoxyribonucleic acid can have one or more bases selected from adenine, thymine, cytosine, and guanine, and a ribonucleic acid can have one or more bases selected from uracil, adenine, cytosine, and guanine Exemplary non-native bases include, but are not limited to, inosine, xanthine, hypoxanthine, isocytosine, isoguanosine, 5-methylcytosine, 5-hydroxymethyl cytosine, 2-aminoadenine, 6-methyl adenine, 6-methyl guanine 2-propyl guanine, 2-propyl adenine, 2-thiothymine, 2-thiocylosine, 5-propynyl uracil, 5-propynyl cytosine, 6-azo uracil, 6-azo cytosine, 6-azo thymine, 4-thiouracil, 8-halo adenine, 8-halo guanine, 8-amino adenine, 8-amino guanine, 8-thiol adenine, 8-thiol guanine, 8-thioalkyl adenine, 8-thioalkyl guanine, 8-hydroxyl adenine, 8-hydroxyl guanine, 5-halo substituted uracil, 5-halo substituted cytosine, 7-methylguanine, 7-methyladenine, 8-azaguanine, 8-azaadenine, 7-deazaguanine, 7-deazaadenine, 3-deazaguanine, and 3-deazaadenine. In certain embodiments, isocytosine and isoguanosine may reduce non-specific hybridization. In some embodiments, a non-native base can have universal base pairing activity, wherein it is capable of base-pairing with any other naturally occurring base, e.g., 3-nitropyrrole and 5-nitroindole.

### Exonuclease Treatment

In some embodiments, cDNAs are treated with an exonuclease (e.g., Exonuclease I) to degrade any primers remaining from the reverse transcription and template switching steps. This prevents possible interference by these primers in subsequent amplification.

### Amplification

As used herein, the term “amplification” or “amplifying” refers to a process by which multiple copies of a particular polynucleotide are formed, and includes methods such as the polymerase chain reaction (PCR), ligation amplification (also known as ligase chain reaction, or LCR), and other amplification methods. In some embodiments, amplification refers specifically to PCR. Amplification methods are widely known in the art. In general, PCR refers to a method of amplification comprising hybridization of primers to specific sequences within a DNA sample and amplification involving multiple rounds of annealing, elongation, and denaturation using a DNA polymerase. The resulting DNA products are then often screened for a band of the correct size. The primers used are oligonucleotides of appropriate length and sequence to provide initiation of polymerization. Reagents and hardware for conducting amplification reactions are widely known and commercially available. Primers useful to amplify sequences from a particular gene region are sufficiently complementary to hybridize to target sequences. Nucleic acids generated by amplification can be sequenced directly.

When hybridization occurs in an antiparallel configuration between two single-stranded polynucleotides, the reaction is called “annealing” and those polynucleotides are described as “complementary”. A double-stranded polynucleotide can be complementary or homologous to another polynucleotide, if hybridization can occur between one of the strands of the first polynucleotide and the second. Complementarity or homology (the degree that one polynucleotide is complementary with another) is quantifiable in terms of the proportion of bases in opposing strands that are expected to form hydrogen bonding with each other, according to generally accepted base-pairing rules. The stringency of hybridization is influenced by hybridization conditions, such as temperature and salt. In the context of amplification, these parameters can be suitably selected.

In some embodiments, cDNA created by reverse transcription and template switching, and optionally treated with an exonuclease, is amplified to provide more starting material for sequencing. cDNA can be amplified by a single primer with a region that is complementary to all cDNAs, e.g., an adapter sequence. Polymerase Mix, such as Advantage 2 Polymerase Mix; and Water, such as nuclease-free water, may be performed using the following program: 95° C. for 1 minute; 18 cycles of a) 95° C. for 15 seconds, 65° C. for 30 seconds, 68° C. for 6 minutes, and 72° C. for 10 minutes (followed by an optional hold period at 4° C.). In certain bulk RNA-seq and lysate sequencing embodiments, this amplification reaction may be modified to use fewer than 18 cycles, e.g., 10 cycles. One exemplary amplification reaction uses 204 of cDNA; 5 μL of 10× Advantage 2 PCR buffer; 1 μL of dNTPs; 1 μL of the DNA primer (10 μM, Integrated DNA Technologies); 1 μL of the Advantage 2 Polymerase Mix; and 22 μL of Nuclease-Free Water, and is optionally performed using the following program: 95° C. for 1 min; 18 cycles of a) 95° C. for 15 sec, 65° C. for 30 sec, 68° C. for 6 min, and 72° C. for 10 min (followed by an option hold period at 4° C.). However, the skilled worker will appreciate that amplification conditions may be adjusted depending on the exact primer and template being used.

### Nucleic Acid Purification and Quantification

Nucleic acid purification (e.g., cDNA purification) is well established. In some embodiments, a nucleic acid (e.g., cDNA) is purified with a spin-based column, such as those commercially available from Zymo Research™ (DNA Clean & Concentrator™-5, Cat. No. D4013) or Qiagen™ (MinElute PCR purification kit. Cat. No. 28004). In particular embodiments, the spin column is a column lacking a physical ring, for example the ring found in Qiagen™ columns, allowing elution of the purified nucleic acid in a lower volume than would be possible in a spin column with a ring. In some embodiments, a nucleic acid (e.g., cDNA, such as in a cDNA library), is purified using magnetic beads. Magnetic bead purification systems are well known and include, for example, the Agencourt AMPure XP™ system (Beckman Coulter, Cat. No. A63881). In some embodiments, a nucleic acid (e.g., cDNA, such as in a cDNA library) is purified after being run on a gel. Gel extraction purification kits are well known, and include, for example, the MinElute Gel Extraction Kit™ (Qiagen, Cat. No. 28604).

### Sequencing Library Preparation

In some embodiments, a cDNA library for sequencing is fragmented prior to the sequencing. A cDNA library can be fragmented by any known method, for example, mechanical fragmentation or a transposase-based fragmentation such as that used in the Nextera™ system (e.g., the Illumina Nextera XT DNA Sample Preparation Kit Cat. No. FC-131-1096 or the Nextera DNA Sample Preparation Kit Cat. No. FC-121-1031). Fragmentation via a transposase-based system has the benefit of being able to incorporate into the fragments barcode sequences that facilitate identification of the fragments. In some embodiments, a barcode sequence introduced during preparation of a cDNA library for sequencing is specific for a predetermined set of cells. This predetermined set of cells can be a subset of a larger set of cells. For example, a tissue biopsy can be sorted into a set of cells to be further sorted into single cells in a capture plate for gene profiling. If a bulk lysate or population of cells is being used as a starting material rather than a single cells that have been sorted, a barcode sequence may, in certain embodiments, not be necessary in this step if a barcode already has been incorporated into the cDNA library in previous steps. However, a plate barcode still could be used to multiplex a high number of samples even for purified RNA/lysates.

### Sequencing Library Quality Assessment

In some embodiments, a cDNA library for sequencing is quantified and evaluated for quality prior to the sequencing to ensure that the library is of sufficient quantity and quality to yield positive results from sequencing. For example, a cDNA library can be quantified using a fluorometer and analyzed for quantity and average size through the use of a number of commercially available kits. The 2 main metrics for quality are the concentration of the library (which needs to be sufficient for loading on the sequencer) and the length of the cDNA fragments to be sequenced. Size selection is performed on a gel to enrich for fragments of the correct size. The gel itself gives an idea of the quality of the library. The final extracted library can be run on an Agilent Bioanalyzer (Cat. No. G2940CA) to obtain the size distribution for the cDNA fragments.

### Sequencing

As used herein, “sequencing” refers to any technique known in the art that allows the identification of consecutive nucleotides of at least part of a nucleic acid. Exemplary sequencing techniques include RNA-seq (also known as whole transcriptome sequencing), Illumina™ sequencing, direct sequencing, random shotgun sequencing, Sanger dideoxy termination sequencing, whole-genome sequencing, massively parallel signature sequencing (MPSS), sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, mass spectrometry, and a combination thereof. In some embodiments, sequencing comprises detecting a sequencing product using an instrument, for example but not limited to an ABI PRISM™ 377 DNA Sequencer, an ABI PRISM™ 310, 3100, 3100-Avant, 3730, or 3730xI Genetic Analyzer, an ABI PRISM™ 3700 DNA Analyzer, or an Applied Biosystems SOLiD™ System (all from Applied Biosystems), a Genome Sequencer 20 System (Roche Applied Science), or a mass spectrometer.

### Plant DNA or Genomic DNA Isolation

The general protocol for isolating plant DNA or genomic DNA may include a variety of RNA containing materials, for example, from plant stems, leaves, roots, seeds and flowers. In some instances, DNA or RNA is isolated from seeds. Plant tissues can be treated with lysis buffer comprising a buffering component to provide a suitable chemical environment for extraction and recovery of RNA analysis. Detailed descriptions of plant DNA or genomic DNA isolation is described in Patent Applicant Publication No: US 20150167053 A1, which is incorporated hereby in its entirety.

Plant DNA or genomic DNA can be isolated and purified using DNeasy Plant Mini Kit (Qiagen, Cat. No/ID: 69104), Plant DNAzol® Reagent (Thermo Fisher Scientific, Cat. No: 10978021), MagMAX™-96 DNA Multi-Sample Kit (Thermo Fisher Scientific, Cat. No: 4413021), MasterPure™ Plant Leaf DNA Purification Kit (Epicenter, Cat. No: MPP92100), QuickExtract™ Plant DNA Extraction Solution (Epicenter, Cat. No: QEP70750), QuickExtract™ Seed DNA Extraction Solution (Epicenter, Cat. No: QES080950), Isolate II plant DNA kit (Bioline, Cat. No: BIO-52068), or ZR Plant/Seed DNA MiniPrep™ (Zymo Research, Cat No: D6020).

Genomic DNA can be isolated by using GenElute™ Plant Genomic DNA Miniprep Kit ((Sigma-Aldrich, G2N10), ChargeSwitch® gDNA Plant Kit (Thermo Fisher Scientific, Cat. No: CS18000), PureLink® Genomic Plant DNA Purification Kit (Thermo Fisher Scientific, Cat No: K183001), Wizard© Genomic DNA Purification Kit (Promega, Cat. No: A1120), and PowerPlant® Pro DNA Isolation Kit (Mobio, Cat. No: 13400-50).

In some embodiments, the plant tissue is treated with lysis buffer comprising buffering component selected from selected from the group consisting of tris(hydroxymethyl)aminomethane (Tris), 4-(2-hydroxyethyl)-1-piperazineethanesulfonic acid (HEPES), 3-(N-morpholino)propanesulfonic acid, (MOPS), Sodium dihydrogen phosphate (NaH₂PO₄), disodium hydrogen phosphate (Na₂HPO₄) and combinations thereof. In various embodiments, the buffering component comprises Tris.

Generally, the buffering component is present at a concentration of at least about 100 mm or at least about 150 mM. Typically, the buffering component is present at a concentration of from about 100 to about 300 mM or from about 150 to about 250 mM.

The lysis buffer may comprise a salt, typically a mineral salt. The salt provides breakdown of cell components to aid in providing DNA for extraction and recovery. The selection of the salt is not narrowly critical and generally any suitable salt known in the art may be utilized. Typically, the salt is a mineral salt selected from the group consisting of sodium chloride (NaCl), potassium chloride (KCl), diammonium sulfate (NH₄SO₄), and combinations thereof. In various embodiments, the mineral salt comprises sodium chloride.

Generally, the (mineral) salt is present at a concentration of at least at about 100 mM, at least about 150 mM, or at least about 200 mM. Typically, the (mineral) salt is present at a concentration of from about 150 to about 350 mM or from about 200 to about 300 mM.

A further component of the extraction buffer of the present invention is a metal chelating agent for the purpose of binding with metal ions present in the extraction buffer that could degrade DNA and, therefore, reduce yields. The selection of the metal chelating agent is not narrowly critical and generally may be selected from those known in the art for use in lysis buffers. Typically, however, the metal chelating agent is selected from the group consisting of ethylenediaminetetraacetic acid (EDTA), ethylene glycol tetraacetic acid (EGTA), and combinations thereof. In various embodiments, the chelating agent comprises EDTA.

Generally, the chelating agent is present at a concentration of at least about 10 mM or at least about 15 mM (e.g., about 25 mM). Typically, the chelating agent is present at a concentration of from about 10 to about 50 mM or from about 15 to about 40 mM.

Various conventional lysis buffers include a surfactant, or detergent component (often referred to as an ionic detergent). The surfactant/detergent is known to disrupt cell walls to release DNA, but also in the case of polysaccharide-rich plant tissues is known to separate polysaccharides from the extracted DNA. Suitable surfactants/detergents include those generally known in the art. Typically, however, the surfactant is selected from the group consisting of sodium dodecyl sulfate (SDS), nonyl phenoxypolyoxylethanol (NP-40), polyethylene glycol p-(1,1,3,3-tetramethylbutyl)-phenyl ether (triton-X), polyoxyethylene (20) sorbitan monooleate (Tween-20), sarkosyl, CTab, and combinations thereof. In various embodiments, the surfactant comprises SDS.

The lysis buffer may include a precipitant. Generally, the cell lysis provides a lysed plant material mixture that comprises a supernatant comprising DNA and a solids fraction. The precipitant contributes to formation of a solids portion (i.e., a “pellet”) in the lysed plant material mixture that includes a significant fraction of impurities, cellular components, etc. and thereby provides a relatively pure DNA sample in the supernatant. Thus, the presence of the precipitant contributes to providing a relatively pure DNA sample. For example, the presence of the precipitant (e.g., glycerol) is currently believed to contribute to sufficient impurity removal such that a filtration step is not required prior to sample recovery. The presence of the precipitant may also avoid the need for cleaning of the DNA sample prior to subsequent analysis (e.g., PCR), or at least reduce the degree of cleaning required to prepare the sample for analysis.

In various embodiments, the precipitant is selected from the group consisting of glycerol, dimethyl sulfoxide (DMSO), acetonitrile (ACN), bovine serum albumin (BSA), proteinase K, acetate salts, and combinations thereof. Suitable acetate salts include, for example, sodium acetate (NaAc) and potassium acetate (KAc). In various embodiments, the bulking agent comprises glycerol.

The precipitant is generally present at a concentration (v/v) of at least about 0.5 wt % of the composition, or at least about 0.75 wt % of the composition. Typically, the precipitant is present at a concentration (v/v) of from about 0.5% to about 1.5%, from about 0.75% to about 1.25%, or about 1%.

In various embodiments, the precipitant is glycerol. For example, the presence of glycerol has been observed to provide extremely pure samples of DNA for analysis. In this regard, glycerol is believed to act as a stabilizing agent, supporting pelleting of sample debris, thereby contributing to a cleaner lysate for DNA analysis. One result observed in connection with the purer sample is improved “clustering” of markers identified in DNA analysis.

The cell lysis buffer may include a higher proportion of surfactant than included in many conventional lysis buffers. For example, in various embodiments the lysis buffer includes greater than 0.5 wt. % of a surfactant (e.g., SDS). This greater proportion of surfactant provides greater disruption of cell walls. It is currently believed that the higher proportion of surfactant provides advantages on its own in this regard. And, as noted above, the presence of the precipitant/glycerol component likewise provides advantages in its own regard. It is currently further believed that the combination of the higher proportion of surfactant and glycerol as a precipitant provides advantageous cell wall disruption while also providing a DNA sample exhibiting suitable DNA purity and yield. That is, the presence of glycerol as a precipitant provides suitable formation of the “pellet” accounting for the greater disruption of cell walls and potential increased release of impurities provided by the greater proportion of surfactant.

The lysis buffer may comprise a polymeric component, which serves to bind with contaminants present in the lysed plant material mixture and thereby have these components present in the solids fraction (i.e., pellet) of the lysed plant material mixture. Contaminants controlled and/or removed by the polymer include polyphenols and polysaccharides. The presence of these contaminants in DNA extraction often renders the sample viscous and results in low DNA yields and/or quality unsatisfactory for downstream analysis. Contaminant control by the polymer provides a relatively pure DNA sample and reduces the need for downstream clean-up prior to subsequent analysis (e.g., restriction endonuclease digestion, polymerase chain reaction (PCR), genotyping and sequencing).

Typically, the lysis buffer comprises a polymer comprising polyvinylpyrrolidone (PVP). In various embodiments, the water-soluble polymer is PVP-10 (commercially available from SIGMA-ALDRICH).

Generally, the polymer is present at a concentration (w/v) of at least about 0.5 wt % of the composition, or at least about 0.75 wt % of the composition. Typically, the polymer is present at a concentration of from about 0.5% to about 1.5%, from about 0.75% to about 1.25%, or about 1%.

Recovery of DNA may involve combining the lysis buffer with the targeted plant material, agitating the mixture of the plant material and lysis buffer to provide a mixture including a supernatant including DNA to be recovered and a solids fraction, and recovering the DNA-containing supernatant.

Combining the lysis buffer and plant material forms a plant material/lysis buffer mixture. Typically, formation of the plant material/lysis buffer mixture includes dilution of the lysis buffer with an aqueous medium (e.g., deionized water).

Generally, an aqueous medium is combined with the lysis buffer at a volumetric ratio (aqueous medium:lysis buffer) of at least about 5:1 or at least about 10:1 for dilution. For example, typically an aqueous medium is combined with the lysis buffer mixture at a volumetric ratio (aqueous medium:lysis buffer) of from about 5:1 to about 20:1, of from about 10:1 to about 20:1, or about 15:1.

After the plant material and lysis buffer have been combined, the mixture is treated to provide breakdown of plant cell walls and release of DNA. Typically, this treatment includes agitation of the plant material/lysis buffer mixture, which generally includes placing samples of the mixture into a suitable container (e.g., a multi-well plate) and shaking of the samples.

In various embodiments, the agitation for breakdown of cell walls and release of DNA includes contacting the plant material with particulate matter for facilitating breakdown of the cell walls. In particular, this contact generally includes placing suitable particulate matter in each well of the multi-well plate so that the particulate matter and plant material come into mutually abrading contact during agitation (e.g., shaking) of the plant material/lysis buffer mixture. The particulate matter is generally spherical and constructed of suitable material (e.g., stainless steel). Since generally spherical, the particulate matter can be considered to be in the form of a “BB.”

After a suitable period of agitation of the plant material/lysis buffer mixture, the resulting mixture generally comprises a lysed plant material mixture including a solids fraction and a supernatant comprising nucleic acid to be recovered. The lysed plant material is then treated for purposes of separating the solids fraction and supernatant. This treatment generally comprises centrifuging the samples (i.e., the multi-well plate) under suitable conditions. Typically, the samples are subjected to treatment by centrifuging at from about 2500 to about 3500 revolutions per minute (rpm) for from about 5 to about 10 minutes.

Prior to agitation of the lysis buffer/plant material mixture, the mixture may be subjected to an incubation period. Generally, any incubation period proceeds for at least about 5 minutes, at least about 10 minutes, or at least about 15 minutes. During the incubation period, the mixture may be subjected to temperatures of room temperature, or even higher. For example, the mixture may be subjected to temperatures of up to about 25° C., up to about 35° C., or up to about 45° C. The precise combination of time/temperature incubation conditions is not narrowly critical, however, in various embodiments, the incubation proceeds for a up to about 15 minutes while the mixture is subjected to a temperature of from about 20° C. to about 30° C. (e.g., about 25° C.).

Separation of the lysed plant material mixture (e.g., by centrifuging) forms a mixture including a nucleic acid supernatant that is then recovered from the lysed plant material mixture. The nucleic acid is then subjected to analysis by any method known in the art, including but not limited to those listed below.

### Nucleic Acid Analysis

The nucleic acids, e.g., cDNA, DNA or genomic DNA, can be utilized for DNA analysis with any established DNA analysis methods. These include marker-assisted breeding studies. These also include genotyping, DNA sequencing, allele specific oligonucleotide probes, hydridization, and single nucleotide polymorphism (SNP) detection. For example, the recovered DNA can be subjected to genotyping by a method selected from the group consisting of polymerase chain reaction (PCR), restriction fragment polymorphism ID of genomic DNA, random amplified polymorphic detection of DNA, and amplified fragment length polymorphism detection.

For PCR analysis, the samples are generally diluted prior to analysis. For example, in the case of leaf plant material, typically the sample is diluted at a ratio of (sample:aqueous medium) from about 1:10 to about 1:100. For chip samples, typically the sample is diluted at a ratio of from about 1:10 to about 1:50. By way of further example, for bulk samples (e.g., for plant material from corn, soy, cotton, canola, and cucumber), the sample is typically diluted at a ratio of from about 1:50 to about 1:1000.

In addition to DNA analysis generally, DNA recovered utilizing the present lysis buffer is suitable for microfluidic DNA analysis conducted generally in accordance with methods known in the art. In various particular embodiments, the recovered DNA is subjected to microfluidic PCR analysis.

While preferred embodiments of the present invention are shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Plant Material

Described herein are systems and methods for enhancing plant performance by expressing a UV-B responsive gene in a plant cell, wherein the UV-B responsive gene is identified by irradiating a plant material with a defined photon energy, wavelength, dose and duration of light source. In some instances, the plant material is irradiated using light having an enriched or supplemented wavelength of UV-B. The UV-B responsive gene may be identified in at least one of a seed, seedling, and a mature plant. The UV-B responsive gene may be associated with improvements in at least one physiological condition such as crop yield and plant performance under biotic stress and abiotic stress.

The plant material may be a harvestable crop. Exemplary harvestable crop include, but are not limited to, lettuce, beans, broccoli, cabbage, carrot, cauliflower, cucumber, melon, onion, peas, peppers, pumpkin, spinach, squash, sweetcorn, tomato, watermelon, alfalfa, canola, corn, maize, kale, cotton, sorghum, soybeans, sugarbeets, cherries, blueberries, blackberries, strawberries, tomatoes, cannabis, and wheat.

The plant material may be from a plant family including, but not limited to, Acanthaceae, Achariaceae, Achatocarpaceae, Acoraceae, Acrobolbaceae, Actinidiaceae, Adelanthaceae, Adiantaceae, Adoxaceae, Aextoxicaceae, Aizoaceae, Akaniaceae, Alismataceae, Allisoniaceae, Alseuosmiaceae, Alstroemeriaceae, Altingiaceae, Alzateaceae, Amaranthaceae, Amaryllidaceae, Amblystegiaceae, Amborellaceae, Anacardiaceae, Anarthriaceae, Anastrophyllaceae, Ancistrocladaceae, Andreaeaceae, Aneuraceae, Anisophylleaceae, Annonaceae, Antheliaceae, Anthocerotaceae, Aphanopetalaceae, Aphloiaceae, Apiaceae, Apocynaceae, Apodanthaceae, Aponogetonaceae, Aquifoliaceae, Araceae, Araliaceae, Araucariaceae, Archidiaceae, Arecaceae, Argophyllaceae, Aristolochiaceae, Arnelliaceae, Asparagaceae, Aspleniaceae, Asteliaceae, Asteropeiaceae, Atherospermataceae, Aulacomniaceae, Austrobaileyaceae, Aytoniaceae, Balanopaceae, Balanophoraceae, Balantiopsaceae, Balsaminaceae, Barbeuiaceae, Barbeyaceae, Bartramiaceae, Basellaceae, Bataceae, Begoniaceae, Berberidaceae, Berberidopsidaceae, Betulaceae, Biebersteiniaceae, Bignoniaceae, Bixaceae, Blandfordiaceae, Blechnaceae, Bonnetiaceae, Boraginaceae, Boryaceae, Boweniaceae, Brachytheciaceae, Brassicaceae, Brevianthaceae, Bromeliaceae, Bruchiaceae, Brunelliaceae, Bruniaceae, Bryaceae, Bryobartramiaceae, Bryoxiphiaceae, Burmanniaceae, Burseraceae, Butomaceae, Buxaceae, Buxbaumiaceae, Byblidaceae, Cabombaceae, Cactaceae, Calceolariaceae, Calomniaceae, Calophyllaceae, Calycanthaceae, Calyceraceae, Calymperaceae, Calypogeiaceae, Campanulaceae, Campyneumataceae, Canellaceae, Cannabaceae, Cannaceae, Capparaceae, Caprifoliaceae, Cardiopteridaceae, Caricaceae, Carlemanniaceae, Caryocaraceae, Caryophyllaceae, Casuarinaceae, Catagoniaceae, Catoscopiaceae, Celastraceae, Centrolepidaceae, Centroplacaceae, Cephalotaceae, Cephalotaxaceae, Cephaloziaceae, Cephaloziellaceae, Ceratophyllaceae, Cercidiphyllaceae, Chaetophyllopsaceae, Chloranthaceae, Chonecoleaceae, Chrysobalanaceae, Cinclidotaceae, Circaeasteraceae, Cistaceae, Cleomaceae, Clethraceae, Cleveaceae, Climaciaceae, Clusiaceae, Colchicaceae, Columelliaceae, Combretaceae, Commelinaceae, Compositae, Connaraceae, Conocephalaceae, Convolvulaceae, Coriariaceae, Cornaceae, Corsiaceae, Corsiniaceae, Corynocarpaceae, Costaceae, Crassulaceae, Crossosomataceae, Cryphaeaceae, Crypteroniaceae, Ctenolophonaceae, Cucurbitaceae, Cunoniaceae, Cupressaceae, Curtisiaceae, Cyatheaceae, Cycadaceae, Cyclanthaceae, Cymodoceaceae, Cynomoriaceae, Cyperaceae, Cyrillaceae, Cyrtopodaceae, Cytinaceae, Daltoniaceae, Daphniphyllaceae, Dasypogonaceae, Datiscaceae, Davalliaceae, Degeneriaceae, Dendrocerotaceae, Dennstaedtiaceae, Diapensiaceae, Dichapetalaceae, Dicksoniaceae, Dicnemonaceae, Dicranaceae, Didiereaceae, Dilleniaceae, Dioncophyllaceae, Dioscoreaceae, Dipentodontaceae, Dipteridaceae, Dipterocarpaceae, Dirachmaceae, Disceliaceae, Ditrichaceae, Doryanthaceae, Droseraceae, Drosophyllaceae, Dryopteridaceae, Ebenaceae, Ecdeiocoleaceae, Echinodiaceae, Elaeagnaceae, Elaeocarpaceae, Elatinaceae, Emblingiaceae, Encalyptaceae, Entodontaceae, Ephedraceae, Ephemeraceae, Equisetaceae, Ericaceae, Eriocaulaceae, Erpodiaceae, Erythroxylaceae, Escalloniaceae, Eucommiaceae, Euphorbiaceae, Euphroniaceae, Eupomatiaceae, Eupteleaceae, Eustichiaceae, Exormothecaceae, Fabroniaceae, Fagaceae, Fissidentaceae, Flagellariaceae, Fontinalaceae, Fontinaliaceae, Fossombroniaceae, Fouquieriaceae, Frankeniaceae, Funariaceae, Garryaceae, Geissolomataceae, Gelsemiaceae, Gentianaceae, Geocalycaceae, Geraniaceae, Gesneriaceae, Gigaspermaceae, Ginkgoaceae, Gisekiaceae, Gleicheniaceae, Gnetaceae, Goebeliellaceae, Gomortegaceae, Goodeniaceae, Goupiaceae, Grammitidaceae, Grimmiaceae, Griseliniaceae, Grossulariaceae, Grubbiaceae, Gunneraceae, Gymnomitriaceae, Gyrostemonaceae, Haemodoraceae, Halophytaceae, Haloragaceae, Hamamelidaceae, Hanguanaceae, Haplomitriaceae, Haptanthaceae, Hedwigiaceae, Heliconiaceae, Helicophyllaceae, Helwingiaceae, Herbertaceae, Hernandiaceae, Himantandraceae, Hookeriaceae, Huaceae, Humiriaceae, Hydatellaceae, Hydnoraceae, Hydrangeaceae, Hydrocharitaceae, Hydroleaceae, Hydrostachyaceae, Hylocomiaceae, Hymenophyllaceae, Hymenophyllopsidaceae, Hymenophytaceae, Hypericaceae, Hypnaceae, Hypnodendraceae, Hypopterygiaceae, Hypoxidaceae, Icacinaceae, Iridaceae, Irvingiaceae, Isoetaceae, Iteaceae, Ixioliriaceae, Ixonanthaceae, Jackiellaceae, Joinvilleaceae, Jubulaceae, Jubulopsaceae, Juglandaceae, Juncaceae, Juncaginaceae, Jungermanniaceae, Kirkiaceae, Koeberliniaceae, Krameriaceae, Lacistemataceae, Lactoridaceae, Lamiaceae, Lanariaceae, Lardizabalaceae, Lauraceae, Lecythidaceae, Leguminosae, Lejeuneaceae, Lembophyllaceae, Lentibulariaceae, Lepicoleaceae, Lepidobotryaceae, Lepidolaenaceae, Lepidoziaceae, Leptodontaceae, Lepyrodontaceae, Leskeaceae, Leucodontaceae, Leucomiaceae, Liliaceae, Limeaceae, Limnanthaceae, Linaceae, Linderniaceae, Loasaceae, Loganiaceae, Lomariopsidaceae, Lophiocarpaceae, Lophocoleaceae, Lophoziaceae, Loranthaceae, Lowiaceae, Loxsomataceae, Lunulariaceae, Lycopodiaceae, Lythraceae, Magnoliaceae, Makinoaceae, Malpighiaceae, Malvaceae, Marantaceae, Marattiaceae, Marcgraviaceae, Marchantiaceae, Marsileaceae, Martyniaceae, Mastigophoraceae, Matoniaceae, Mayacaceae, Meesiaceae, Melanthiaceae, Melastomataceae, Meliaceae, Melianthaceae, Menispermaceae, Menyanthaceae, Mesoptychiaceae, Metaxyaceae, Meteoriaceae, Metteniusaceae, Metzgeriaceae, Misodendraceae, Mitrastemonaceae, Mitteniaceae, Mniaceae, Molluginaceae, Monimiaceae, Monocarpaceae, Montiaceae, Montiniaceae, Moraceae, Moringaceae, Muntingiaceae, Musaceae, Myliaceae, Myodocarpaceae, Myricaceae, Myriniaceae, Myristicaceae, Myrothamnaceae, Myrtaceae, Myuriaceae, Nartheciaceae, Neckeraceae, Nelumbonaceae, Neotrichocoleaceae, Nepenthaceae, Neuradaceae, Nitrariaceae, Nothofagaceae, Notothyladaceae, Nyctaginaceae, Nymphaeaceae, Ochnaceae, Octoblepharaceae, Oedipodiaceae, Olacaceae, Oleaceae, Oleandraceae, Onagraceae, Oncothecaceae, Ophioglossaceae, Opiliaceae, Orchidaceae, Orobanchaceae, Orthorrhynchiaceae, Orthotrichaceae, Osmundaceae, Oxalidaceae, Oxymitraceae, Paeoniaceae, Pallaviciniaceae, Pandaceae, Pandanaceae, Papaveraceae, Paracryphiaceae, Passifloraceae, Paulowniaceae, Pedaliaceae, Pelliaceae, Penaeaceae, Pentadiplandraceae, Pentaphragmataceae, Pentaphylacaceae, Penthoraceae, Peraceae, Peridiscaceae, Petermanniaceae, Petrosaviaceae, Philesiaceae, Philydraceae, Phrymaceae, Phyllanthaceae, Phyllodrepaniaceae, Phyllogoniaceae, Phyllonomaceae, Physenaceae, Phytolaccaceae, Picramniaceae, Picrodendraceae, Pilotrichaceae, Pinaceae, Piperaceae, Pittosporaceae, Plagiochilaceae, Plagiogyriaceae, Plagiotheciaceae, Plantaginaceae, Platanaceae, Pleuroziaceae, Pleuroziopsaceae, Plocospermataceae, Plumbaginaceae, Poaceae, Podocarpaceae, Podostemaceae, Polemoniaceae, Polygalaceae, Polygonaceae, Polypodiaceae, Polytrichaceae, Pontederiaceae, Porellaceae, Portulacaceae, Posidoniaceae, Potamogetonaceae, Pottiaceae, Primulaceae, Prionodontaceae, Proteaceae, Pseudolepicoleaceae, Psilotaceae, Pteridaceae, Pterigynandraceae, Pterobryaceae, Ptilidiaceae, Ptychomitriaceae, Ptychomniaceae, Putranjivaceae, Quillajaceae, Racopilaceae, Radulaceae, Rafflesiaceae, Ranunculaceae, Rapateaceae, Regmatodontaceae, Resedaceae, Restionaceae, Rhabdodendraceae, Rhabdoweisiaceae, Rhachitheciaceae, Rhacocarpaceae, Rhamnaceae, Rhipogonaceae, Rhizogoniaceae, Rhizophoraceae, Ricciaceae, Riellaceae, Rigodiaceae, Roridulaceae, Rosaceae, Rousseaceae, Rubiaceae, Ruppiaceae, Rutaceae, Rutenbergiaceae, Sabiaceae, Salicaceae, Salvadoraceae, Salviniaceae, Santalaceae, Sapindaceae, Sapotaceae, Sarcobataceae, Sarcolaenaceae, Sarraceniaceae, Saururaceae, Saxifragaceae, Scapaniaceae, Scheuchzeriaceae, Schisandraceae, Schistochilaceae, Schistostegaceae, Schizaeaceae, Schlegeliaceae, Schoepfiaceae, Scorpidiaceae, Scrophulariaceae, Selaginellaceae, Seligeriaceae, Sematophyllaceae, Serpotortellaceae, Setchellanthaceae, Simaroubaceae, Simmondsiaceae, Siparunaceae, Sladeniaceae, Smilacaceae, Solanaceae, Sphaerosepalaceae, Sphagnaceae, Sphenocleaceae, Spiridentaceae, Splachnaceae, Splachnobryaceae, Stachyuraceae, Staphyleaceae, Stegnospermataceae, Stemonaceae, Stemonuraceae, Stereophyllaceae, Stilbaceae, Strasburgeriaceae, Strelitziaceae, Stylidiaceae, Styracaceae, Surianaceae, Symplocaceae, Takakiaceae, Talinaceae, Tamaricaceae, Tapisciaceae, Targioniaceae, Taxaceae, Taxodiaceae, Tecophilaeaceae, Tetrachondraceae, Tetramelaceae, Tetrameristaceae, Tetraphidaceae, Thamnobryaceae, Theaceae, Theliaceae, Thelypteridaceae, Thomandersiaceae, Thuidiaceae, Thurniaceae, Thymelaeaceae, Ticodendraceae, Timmiaceae, Tofieldiaceae, Torricelliaceae, Tovariaceae, Trachypodaceae, Treubiaceae, Trichocoleaceae, Trigoniaceae, Triuridaceae, Trochodendraceae, Tropaeolaceae, Typhaceae, Ulmaceae, Urticaceae, Vahliaceae, Velloziaceae, Verbenaceae, Vetaformaceae, Violaceae, Vitaceae, Vittariaceae, Vivianiaceae, Vochysiaceae, Wardiaceae, Welwitschiaceae, Wiesnerellaceae, Winteraceae, Woodsiaceae, Xanthorrhoeaceae, Xeronemataceae, Xyridaceae, Zamiaceae, Zingiberaceae, Zosteraceae, Zygophyllaceae. In some instances, the plant family is at least one of Brassicaceae, Poaceae, Solanaceae, Fabaceae, Labiaceae, Rosaceae, and Asteraceae (or Compositae).

### Numbered Embodiments

Numbered embodiment 1 comprises a method for identifying a modulator of a UVR8-COP1-HY5 UV-B signaling pathway that improves growth in a plant, the method comprising: irradiating plant material using light having an enriched wavelength between 280-320 nm; selecting the plant material having at least one physiological condition selected from the group consisting of enhanced crop yield, growth rate, hardiness, stress resistance, root growth, root architecture, and pathological resistance compared to a plant material lacking the irradiation; and identifying a gene that is associated with the at least one physiological condition. Numbered embodiment 2 comprises the method of numbered embodiment 1, wherein the plant material is exposed to an enriched wavelength of about 286 nm. Numbered embodiment 3 comprises the method of numbered embodiments 1-2, wherein the plant material is exposed to an enriched wavelength of 286 nm prior to a subsequent growth phase of a seedling. Numbered embodiment 4 comprises the method of numbered embodiments 1-3, wherein the plant material is exposed to an enriched wavelength of about 280 nm. Numbered embodiment 5 comprises the method of numbered embodiments 1-4, wherein the plant material is exposed to an enriched wavelength of 280 nm prior to a subsequent growth phase of a seedling. Numbered embodiment 6 comprises the method of numbered embodiments 1-5, further comprising determining a nucleic acid sequence of the gene that is associated with the at least one physiological condition. Numbered embodiment 7 comprises the method of numbered embodiments 1-6, wherein the determining comprises at least one of nucleic acid sequencing, microarray, quantitative-polymerase chain reaction, Western blot, and immunohistochemistry analysis. Numbered embodiment 8 comprises the method of numbered embodiments 1-7, wherein the root architecture comprises at least one of nodule formation, root growth, and spatial configuration. Numbered embodiment 9 comprises the method of numbered embodiments 1-8, further comprising generating a transgenic plant comprising the gene that is associated with the at least one physiological condition. Numbered embodiment 10 comprises the method of numbered embodiments 1-9, wherein the gene that is associated with the at least one physiological condition is selected from a group of genes consisting of HY5, CHS, COP1, UVR8, HYH, GPX7, SIG5, CRY3, ELIP1, SWA3, PHYA, FAR1, FHY3, FHY1FHL, MYB111, MYB12, MKP1, PAP1, C4H, MYB4, AtMYB12, AtCHS, and AtC4H. Numbered embodiment 11 comprises the method of numbered embodiments 1-10, wherein the gene that is associated with the at least one physiological condition is a modulator of the UVR8-COP1-HY5 UV-B signaling pathway. Numbered embodiment 12 comprises the method of numbered embodiments 1-11, wherein the gene that is associated with the at least one physiological condition when expressed activates a downstream regulator of the UVR8-COP1-HY5 UV-B signaling pathway. Numbered embodiment 13 comprises the method of numbered embodiments 1-12, wherein the gene that is associated with the at least one physiological condition when expressed increases a gene of the UVR8-COP1-HY5 UV-B signaling pathway. Numbered embodiment 14 comprises the method of numbered embodiments 1-13, wherein the gene that is associated with the at least one physiological condition when expressed reduces a suppressor of the UVR8-COP1-HY5 UV-B signaling pathway. Numbered embodiment 15 comprises the method of numbered embodiments 1-14, wherein the gene that is associated with the at least one physiological condition is UVR8. Numbered embodiment 16 comprises the method of numbered embodiments 1-15, the gene that is associated with the at least one physiological condition is COP1. Numbered embodiment 17 comprises the method of numbered embodiments 1-16, wherein the gene that is associated with the at least one physiological condition is HY5. Numbered embodiment 18 comprises the method of numbered embodiments 1-17, wherein the gene that is associated with the at least one physiological condition is CHS. Numbered embodiment 19 comprises the method of numbered embodiments 1-18, wherein the gene that is associated with the at least one physiological condition is expressed in at least one of seed, seedling, fruit, ovule, carpel, embryo, pericarp, endosperm, pollen, root, leaf, stem, and flower. Numbered embodiment 20 comprises the method of numbered embodiments 1-19, wherein the gene that is associated with the at least one physiological condition modulates a downstream responsive gene expressed in at least one of seed, seedling, fruit, ovule, carpel, embryo, pericarp, endosperm, pollen, root, leaf, stem, and flower. Numbered embodiment 21 comprises the method of numbered embodiments 1-20, wherein the gene that is associated with the at least one physiological condition when activated increases expression of at least one of flavonoid, anthocyanin, ascorbate acid, and tocopherol. Numbered embodiment 22 comprises the method of numbered embodiments 1-21, wherein the gene that is associated with the at least one physiological condition when activated increases expression of flavonoid. Numbered embodiment 23 comprises the method of numbered embodiments 1-22, wherein the gene that is associated with the at least one physiological condition when activated increases expression of anthocyanin. Numbered embodiment 24 comprises the method of numbered embodiments 1-23, wherein the gene that is associated with the at least one physiological condition modulates expression of a plant hormone selected from the group consisting of auxins, gibberellins, cytokinins, and brassinosteroids. Numbered embodiment 25 comprises the method of numbered embodiments 1-24, wherein the gene that is associated with the at least one physiological condition modulates expression of a plant ripening hormone. Numbered embodiment 26 comprises the method of numbered embodiments 1-25, wherein the gene that is associated with the at least one physiological condition modulates expression of a seed germination hormone. Numbered embodiment 27 comprises the method of numbered embodiments 1-26, wherein improvement of the physiological condition is characterized by an increase in at least one of dry weight, shoot fresh weight, pigment production, radical length, leaf size, and nitrogen index. Numbered embodiment 28 comprises the method of numbered embodiments 1-27, wherein the physiological condition is enhanced by at least 5%. Numbered embodiment 29 comprises the method of numbered embodiments 1-28, wherein the physiological condition is enhanced by at least 10%. Numbered embodiment 30 comprises the method of numbered embodiments 1-29, wherein the physiological condition is enhanced by at least 30%. Numbered embodiment 31 comprises the method of numbered embodiments 1-30, wherein the physiological condition is enhanced by at least 50%. Numbered embodiment 32 comprises the method of numbered embodiments 1-31, wherein the plant material is selected from the group consisting of lettuce, beans, broccoli, cabbage, carrot, cauliflower, cucumber, melon, onion, peas, peppers, pumpkin, spinach, kale, squash, sweetcorn, corn, maize, tomato, watermelon, alfalfa, canola, cotton, sorghum, soybeans, sugar beets, wheat, rice, and a grass. Numbered embodiment 33 comprises the method of numbered embodiments 1-32, wherein the plant material is an indoor plant. Numbered embodiment 34 comprises the method of numbered embodiments 1-33, wherein the plant material is an outdoor plant. Numbered embodiment 35 comprises the method of numbered embodiments 1-34, wherein the plant material comprises at least one of a seed, a seedling, and a mature plant. Numbered embodiment 36 comprises a transgenic plant comprising an isolated polynucleotide comprising a nucleic acid sequence encoding a modulator that is responsive to UV-B administration in a plant material of the transgenic plant, wherein the transgenic plant produces at least one enhanced phenotype in the absence of the supplementary UV-B irradiation, and wherein the at least one enhanced phenotype is selected from the group consisting of increased crop yield, growth rate, hardiness, stress resistance, and pathological resistance when compared to a plant lacking the modulator. Numbered embodiment 37 comprises the transgenic plant of numbered embodiments 1-36, wherein the modulator is a modulator of the UVR8-COP1-HY5 UV-B signaling pathway. Numbered embodiment 38 comprises the transgenic plant of numbered embodiments 1-37, wherein the modulator is selected from a group of genes consisting of Hy5, CHS, COP1, UVR8, HYH, GPX7, SIG5, CRY3, ELIP1, SWA3, PHYA, FAR1, FHY3, FHY1FHL, MYB111, MYB12, MKP1, PAP1, C4H, MYB4, AtMYB12, AtCHS, and AtC4H. Numbered embodiment 39 comprises the transgenic plant of numbered embodiments 1-38, wherein the modulator when expressed activates a downstream regulator of the UVR8-COP1-HY5 UV-B signaling pathway. Numbered embodiment 40 comprises the transgenic plant of numbered embodiments 1-39, wherein the modulator when expressed increases accumulation of a transcript encoding a member of the UVR8-COP1-HY5 UV-B signaling pathway. Numbered embodiment 41 comprises the transgenic plant of numbered embodiments 1-40, wherein the modulator when expressed reduces a suppressor of UVR8-COP1-HY5 UV-B signaling pathway. Numbered embodiment 42 comprises the transgenic plant of numbered embodiments 1-41, wherein the modulator is UVR8. Numbered embodiment 43 comprises the transgenic plant of numbered embodiments 1-42, wherein the modulator is COP1. Numbered embodiment 44 comprises the transgenic plant of numbered embodiments 1-43, wherein the modulator is HY5. Numbered embodiment 45 comprises the transgenic plant of numbered embodiments 1-44, wherein the modulator is CHS. Numbered embodiment 46 comprises the transgenic plant of numbered embodiments 1-45, further comprising a transgenic tissue-specific promoter. Numbered embodiment 47 comprises the transgenic plant of numbered embodiments 1-46, wherein the tissue-specific promoter comprises at least one of a fruit, ovule-, carpel-, embryo-, pericarp-, endosperm-, pollen-, root-, leaf-, stem-, and a flower-specific promoter. Numbered embodiment 48 comprises the transgenic plant of numbered embodiments 1-47, further comprising a polynucleotide for increasing a level of an endogenous plant hormone. Numbered embodiment 49 comprises the transgenic plant of numbered embodiments 1-48, wherein the plant hormone is selected from the group consisting of auxins, gibberellins, cytokinins, and brassinosteroids. Numbered embodiment 50 comprises the transgenic plant of numbered embodiments 1-49, further comprising a promoter for expressing a polynucleotide in the presence of a plant hormone. Numbered embodiment 51 comprises the transgenic plant of numbered embodiments 1-50, wherein the plant hormone is selected from the group consisting of auxins, gibberellins, cytokinins, and brassinosteroids. Numbered embodiment 52 comprises the transgenic plant of numbered embodiments 1-51, further comprising a promoter specific for expressing a polynucleotide during fruit ripening. Numbered embodiment 53 comprises the transgenic plant of numbered embodiments 1-52, further comprising a promoter specific for expressing a polynucleotide during seed germination. Numbered embodiment 54 comprises the transgenic plant of numbered embodiments 1-53, further comprising a constitutive promoter. Numbered embodiment 55 comprises the transgenic plant of numbered embodiments 1-54, wherein the transgenic plant has improvement of a physiological condition characterized by an increase in at least one of dry weight, shoot fresh weight, pigment production, radical length, leaf size, and nitrogen index. Numbered embodiment 56 comprises the transgenic plant of numbered embodiments 1-55, wherein the phenotype is enhanced by at least 5%. Numbered embodiment 57 comprises the transgenic plant of numbered embodiments 1-56, wherein the phenotype is enhanced by at least 10%. Numbered embodiment 58 comprises the transgenic plant of numbered embodiments 1-57, wherein the phenotype is enhanced by at least 30%. Numbered embodiment 59 comprises the transgenic plant of numbered embodiments 1-58, wherein the phenotype is enhanced by at least 50%. Numbered embodiment 60 comprises the transgenic plant of numbered embodiments 1-59, wherein the transgenic plant is selected from the group consisting of lettuce, beans, broccoli, cabbage, carrot, cauliflower, cucumber, melon, onion, peas, peppers, pumpkin, spinach, kale, squash, sweetcorn, corn, maize, tomato, watermelon, alfalfa, canola, cotton, sorghum, soybeans, sugar beets, wheat, rice, grass, and flowering plants. Numbered embodiment 61 comprises the transgenic plant of numbered embodiments 1-60, wherein the transgenic plant is an indoor plant. Numbered embodiment 62 comprises the transgenic plant of numbered embodiments 1-61, wherein the transgenic plant is an outdoor plant. Numbered embodiment 63 comprises the transgenic plant of numbered embodiments 1-62, wherein the plant material comprises at least one of a seed, a seedling, and a plant. Numbered embodiment 64 comprises a method of generating a transgenic plant having at least one of improved plant performance and improved hardiness, comprising transforming a UV-B responsive gene into a wildtype plant cell, wherein the UV-B responsive gene is responsive to light enriched for UV-B in a range of about 281 nm to about 291 nm. Numbered embodiment 65 comprises a method of numbered embodiments 1-64, wherein the improved plant performance is selected from a group consisting of fruit fresh weight, number of fruit harvested, Brix content, fruit width, fruit length, leaf size, leaf surface area, dry weight, nitrogen content, shoot dry weight, shoot fresh weight, root dry weight, vegetable development, yield of fruiting parts, weight of fruiting parts, hardiness, root growth, root architecture, and seed germination rate. Numbered embodiment 66 comprises a method of numbered embodiments 1-65, wherein the root architecture comprises at least one of nodule formation, root growth, and spatial configuration. Numbered embodiment 67 comprises a method of numbered embodiments 1-66, wherein the improved hardiness is selected from a group consisting of an improved resistance to stress caused by weather damage, an improved resistance to stress caused by sun exposure, an improved resistance to stress caused by disease, and an improved resistance to stress caused by insects. Numbered embodiment 68 comprises a method of numbered embodiments 1-67, wherein the UV-B responsive gene is responsive to UV-B peaking at 286 nm. Numbered embodiment 69 comprises a method of numbered embodiments 1-68, wherein the UV-B responsive gene is responsive to UV-B having an irradiance up to 1.3×10⁻⁴ W cm⁻² s⁻¹. Numbered embodiment 70 comprises a method of numbered embodiments 1-69, wherein the UV-B responsive gene is responsive to UV-B having a dose of no more than 100 kJ m⁻². Numbered embodiment 71 comprises a method of numbered embodiments 1-70, wherein the UV-B responsive gene is selected from a group consisting of HY5, CHS, COP1, UVR8, HYH, GPX7, SIG5, CRY3, ELIP1, SWA3, PHYA, FAR1, FHY3, FHY1FHL, MYB111, MYB12, MKP1, PAP1, C4H, MYB4, AtMYB12, AtCHS, and AtC4H. Numbered embodiment 72 comprises a method of numbered embodiments 1-71, wherein the UV-B responsive gene is a modulator of the UVR8-COP1-HY5 UV-B signaling pathway. Numbered embodiment 73 comprises a method of numbered embodiments 1-72, wherein the UV-B responsive gene is UVR8. Numbered embodiment 74 comprises a method of numbered embodiments 1-73, wherein the UV-B responsive gene is COP1. Numbered embodiment 75 comprises a method of numbered embodiments 1-74, wherein the UV-B responsive gene is HY5. Numbered embodiment 76 comprises a method of numbered embodiments 1-75, wherein the UV-B responsive gene is CHS. Numbered embodiment 77 comprises a method of numbered embodiments 1-76, wherein the at least one of improved plant performance and improved hardiness is enhanced by at least 5%. Numbered embodiment 78 comprises a method of numbered embodiments 1-77, wherein the at least one of improved plant performance and improved hardiness is enhanced by at least 10%. Numbered embodiment 79 comprises a method of numbered embodiments 1-78, wherein the at least one of improved plant performance and improved hardiness is enhanced by at least 30%. Numbered embodiment 80 comprises a method of numbered embodiments 1-79, wherein the plant is selected from the group consisting of lettuce, beans, broccoli, cabbage, carrot, cauliflower, cucumber, melon, onion, peas, peppers, pumpkin, spinach, kale, squash, sweetcorn, corn, maize, tomato, watermelon, alfalfa, canola, cotton, sorghum, soybeans, sugar beets, wheat, rice, and a grass. Numbered embodiment 81 comprises a method of numbered embodiments 1-80, wherein the UV-B responsive gene is mutated. Numbered embodiment 82 comprises a method of numbered embodiments 1-81, wherein the UV-B responsive gene is mutated using methods comprising at least one of CRISPR, zinc finger nucleases, and transcription activator-like effector nucleases. Numbered embodiment 83 comprises a method of numbered embodiments 1-82, wherein the plant cell comprises at least one of a seed cell, a seedling cell, and a mature plant cell. Numbered embodiment 84 comprises a transgenic plant comprising a UV-B responsive gene, wherein the UV-B responsive gene is responsive to light enriched for UV-B in a range of about 281 nm to about 291 nm. Numbered embodiment 85 comprises a transgenic plant of numbered embodiments 1-84, wherein the UV-B responsive gene is mutated. Numbered embodiment 86 comprises a transgenic plant of numbered embodiments 1-85, wherein the UV-B responsive gene is mutated using methods comprising at least one of CRISPR, zinc finger nucleases, and transcription activator-like effector nucleases. Numbered embodiment 87 comprises a transgenic plant of numbered embodiments 1-86, wherein the UV-B responsive gene is responsive to UV-B peaking at 286 nm. Numbered embodiment 88 comprises a transgenic plant of numbered embodiments 1-87, wherein the UV-B responsive gene is responsive to UV-B having an irradiance up to 1.3×10-4 W cm⁻² s⁻¹. Numbered embodiment 89 comprises a transgenic plant of numbered embodiments 1-88, wherein the UV-B responsive gene is responsive to UV-B having a dose of no more than 100 kJ m⁻². Numbered embodiment 90 comprises a transgenic plant of numbered embodiments 1-89, wherein the UV-B responsive gene is selected from a group consisting of HY5, CHS, COP1, UVR8, HYH, GPX7, SIG5, CRY3, ELIP1, SWA3, PHYA, FAR1, FHY3, FHY1FHL, MYB111, MYB12, MKP1, PAP1, C4H, MYB4, AtMYB12, AtCHS, and AtC4H. Numbered embodiment 91 comprises a transgenic plant of numbered embodiments 1-90, wherein the UV-B responsive gene is a modulator of the UVR8-COP1-HY5 UV-B signaling pathway. Numbered embodiment 92 comprises a transgenic plant of numbered embodiments 1-91, wherein the UV-B responsive gene is UVR8. Numbered embodiment 93 comprises a transgenic plant of numbered embodiments 1-92, wherein the UV-B responsive gene is COP1. Numbered embodiment 94 comprises a transgenic plant of numbered embodiments 1-93, wherein the UV-B responsive gene is HY5. Numbered embodiment 95 comprises a transgenic plant of numbered embodiments 1-94, wherein the UV-B responsive gene is CHS. Numbered embodiment 96 comprises a transgenic plant of numbered embodiments 1-95, wherein the transgenic plant comprises improved plant performance. Numbered embodiment 97 comprises a transgenic plant of numbered embodiments 1-96, wherein the improved plant performance is selected from a group consisting of fruit fresh weight, number of fruit harvested, Brix content, fruit width, fruit length, leaf size, leaf surface area, dry weight, nitrogen content, shoot dry weight, shoot fresh weight, root dry weight, vegetable development, yield of fruiting parts, weight of fruiting parts, hardiness, root growth, root architecture, and seed germination rate. Numbered embodiment 98 comprises a transgenic plant of numbered embodiments 1-97, wherein the root architecture comprises at least one of nodule formation, root growth, and spatial configuration. Numbered embodiment 99 comprises a transgenic plant of numbered embodiments 1-98, wherein the transgenic plant comprises improved hardiness. Numbered embodiment 100 comprises a transgenic plant of numbered embodiments 1-99, wherein the improved hardiness is selected from a group consisting of an improved resistance to stress caused by weather damage, an improved resistance to stress caused by sun exposure, an improved resistance to stress caused by disease, and an improved resistance to stress caused by insects. Numbered embodiment 101 comprises a transgenic plant of numbered embodiments 1-100, wherein the plant is selected from the group consisting of lettuce, beans, broccoli, cabbage, carrot, cauliflower, cucumber, melon, onion, peas, peppers, pumpkin, spinach, kale, squash, sweetcorn, corn, maize, tomato, watermelon, alfalfa, canola, cotton, sorghum, soybeans, sugar beets, wheat, rice, and a grass. Numbered embodiment 102 comprises a method of reducing environmental impact of growing a crop, comprising the steps of: sowing a seed comprising a UV-B responsive gene, wherein the UV-B responsive gene is responsive to light enriched for UV-B in a range of about 281 nm to about 291 nm; sowing the seed; providing no more than at least one of a standard fertilizer regimen, a standard pesticide regimen, a standard herbicide regimen, and a standard insecticide regimen; and harvesting the crop from said seed, wherein a crop yield of the crop from said seed is at least 5% greater than a standard yield. Numbered embodiment 103 comprises a transgenic plant of numbered embodiments 1-102, wherein the transgenic plant is a seed. Numbered embodiment 104 comprises a transgenic plant of numbered embodiments 1-103, wherein the transgenic plant is grown from a transgenic seed. Numbered embodiment 104 comprises a transgenic seed comprising an isolated polynucleotide comprising a nucleic acid sequence encoding a modulator that is responsive to UV-B administration, wherein the transgenic seed produces at least one enhanced phenotype in the absence of the supplementary UV-B irradiation, and wherein the at least one enhanced phenotype is selected from the group consisting of increased crop yield, growth rate, hardiness, stress resistance, and pathological resistance when compared to a seed lacking the modulator.

### EXAMPLES

### Example 1: Priming the Seeds

This example illustrates procedures for preparing, handling seeds prior to UV treatment and treating the seeds with UV.

Seeds from a plant, e.g. pioneer maize (P0021), are generally stored in a Tupperware container in the seed fridge. About 1000 seeds are selected. The selected seeds are washed under cold water (FIG. 1). In some cases, washing the seed removes red fungicide coating that may present on the seeds. The seeds are dried with a paper towel.

The washed and dried seed are arranged with the seed embryo-side up into seed dishes. The embryo-side are arranged to face toward the light source. The seeds are split across as many dishes as possible so as to reduce pseudo-replication (FIG. 2). The seeds are kept in a refrigerator for overnight. The seeds may be kept at about 25° C. The seeds may be kept at a relative humidity at about 95%.

The seeds are divided into five groups as described in Table 2.

TABLE 2
UV-B treatment in seeds

| Group 1 | Group 2 | Group 3 | Group 4 | |
|---|---|---|---|---|
| No priming + | Priming + | Priming + | Priming + | Priming + |
| No UV | UV-B* | No UV-B | UV-B* | No UV-B |
| | (13 kJ m⁻²) | (13 kJ m⁻²) | (100 kJ m⁻²) | (100 kJ m⁻²) |

The seeds are arranged in rows and placed under LED panels (FIG. 3), which provides the desired UV-B wavelength. The UV is set to have photon energy about 3.94-4.43 eV, or about 0.631-0.71 aJ. The UV is set to have irradiance in a range between 4×10⁻⁵ to 1.3×10⁻⁴ wcms⁻¹. The UV-B wavelength is about 280±5 nm. The UV-B can be at a dose in a range between 20-80 mA. The UV-B is set at a dose about 13 kJ m⁻² or about 100 kJ m⁻².

The seeds are left for imbibition for about 16 hours prior to treatment. The seeds are treated with UV about 9 hours and 21 hours.

After treatment, the seeds are dried to remove excess water. In some cases, the seeds are left to air dry for about 72 hours. The dried seeds may be uncovered and stored in a container.

The priming process is operated by a control system connected to software, internet, or an electronic device, e.g., a computer. The control system may comprise a Raspberry Pie controller with wifi and zigbee module attached (FIG. 4).

After priming and UV-B treatment, the seeds are sowed systematically or randomly. The procedures for sowing the seeds may involve preparing a randomized sowing key in excel. The number of replicates per treatment may be determined and a list of all replicates may be created (FIG. 5). For example, the random number of every cell adjacent cell is assigned by using=rand( ) in excel. The replicates may be sorted by the random numbers, which may shuffle the list of replicates. The shuffled list of replicates may be used to create a randomized 12×12 sowing key (FIG. 6). The seeds may be sowed in a 12×12 tray accordingly to the 12×12 sowing key.

Once the seeds germinate, dualex is performed to assess flavonol, anthocyanin and chlorophyll contents in the leaves. Dualex allows for performing real-time and non-destructive measurements. The assessment of polyphenolic compounds in leaves is based on the absorbance of the leaf epidermis through the screening effect it procures to chlorophyll fluorescence. Typically, the indices calculated by Dualex are: (i) Anth, for the anthocyanin index; (ii) Chl, for the chlorophyll index; (iii) Flav, for the flavonols index; and (iv) NBI, for the nitrogen balance index.

Dualex is completed as soon as the cotyledons are big enough, for example, on day 5 after priming the seeds.

Seedlings are harvested by 21 days old or by stage V2. Fresh shoot, leaf, and root are collected and their fresh weights are measured. In addition, dried weights of the collected shoot, leaf and root are measured for further analysis.

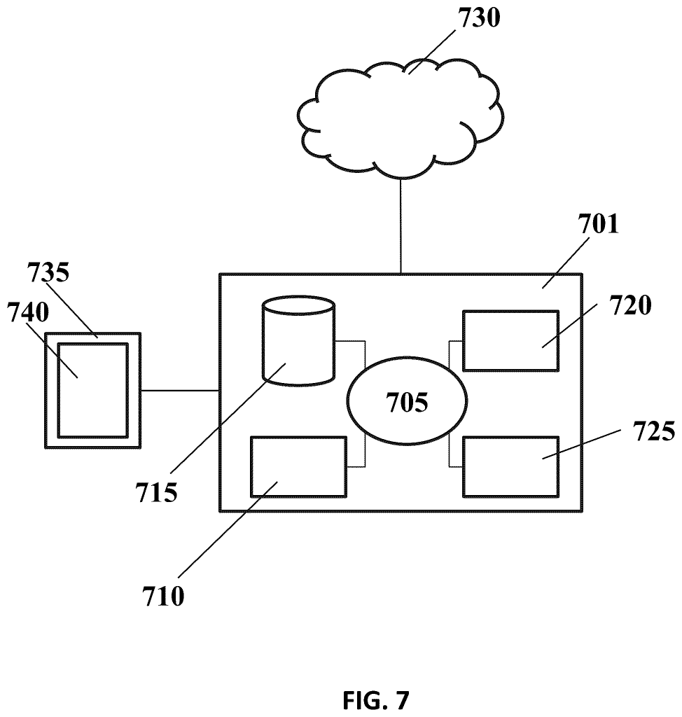

In some embodiments, control of the light source and randomization of the sowing key are performed in a computer system. The computer system 700 illustrated in FIG. 7 may be understood as a logical apparatus that can read instructions from media 711 and/or a network port 705, which is optionally connected to server 709 having fixed media 712. In some cases, the system, such as shown in FIG. 7 includes a CPU 701, disk drives 703, optional input devices such as keyboard 715 and/or mouse 716 and optional monitor 707. In certain cases, data communication is achieved through the indicated communication medium to a server at a local or a remote location. In further cases, the communication medium includes any means of transmitting and/or receiving data. In some cases, the communication medium is a network connection, a wireless connection or an internet connection. In certain examples, such a connection provides for communication over the World Wide Web. It is envisioned that data relating to the present disclosure can be transmitted over such networks or connections for reception and/or review by a party 722 as illustrated in FIG. 7.

### Example 2: UV-B Treatment in Seeds or Seed Embryos have Increased Resistance to Downy Mildew Symptoms

This example illustrates the beneficial effects of UV-B treatment in seeds or seed embryos prior to sowing. Plants grown from seeds or seed embryos are treated with the UV-B regimen described in Example 1 exhibits higher resistance to downy mildew symptom when compared to their non-UB-B treatment counterparts.

Seeds of lettuce are prepared and sowed as described in Example 1. Seeds are divided into five groups as shown in Table 3.

TABLE 3
UV-B treatment regimen for lettuce seeds

| Group 1 | Group 2 | Group 3 | Group 4 | Group 5 |
|---|---|---|---|---|
| No priming, | Priming + | Priming + | Priming + | Priming + |
| No UV | UV-B* | No UV-B | UV-B | No UV-B |
| | (13 kJ m⁻²) | (13 kJ m⁻²) | (100 kJ m⁻²) | (100 kJ m⁻²) |

*UV-B wavelength ~280 nm, treatment time is 9 h at 13 kJ, or 21 h at 21 kJ.

The seedlings are allowed to grow to maturity. Plants are inspected for vitality and growth performance regularly. In general, plants are inspected for non-vigorous with yellowish or pale green foliage, mild or inconspicuous mottling, stunting of plant growth, downward curling or distortion of the leaves, loss of leaves, wilting, white to light gray downy “fuzz” on the undersides of the leaves, or plant collapse.

Plants in Groups 1, 3, and 5 are more susceptive to infection and display downy mildew symptoms ranging from mild symptom such as yellowish foliage to severe symptom such as loss of leaves. Plants in Groups 2 and 4 have higher resistance to downy mildew symptom. These plants appear healthy, and show increased hardiness, increased height, or increase leaf surface. Plants in Group 2 appear to have higher growth rate with increased height and leaf surface by at least 5% when compared with Group 4 plants, which received a different dose of UV-B from the Group 2 plants.

The results demonstrate that UV-B treated in seeds or seed embryos provides beneficial effect to plants in enhancing the plant growth and/or immunological defense against infections. The results also demonstrate that a UV-B dose between 13-100 kJ m⁻² is sufficient to provide protection in plants. The results show that a particular of UV-B dose, e.g., a low dose at 13 kJ m⁻² for 9 hours, when exposed to plant seeds prior to sowing provides better protection in plants. This effect; however, may vary when the duration of UV-B treatment is decreased or increased.

### Example 3: UV-B Treatment in Seeds/Seed Embryos Increases Plant Dry Weight

This example illustrates the beneficial effects of UV-B treatment in seeds or seed embryos prior to sowing. Plants grown from seeds or seed embryos are handled and prepared for UV-B treatment as described in Example 1 exhibits enhanced growth or increased dry weight when compared to their non-UB-B treatment counterparts.

Seeds of maize (Zea mays var. NZ yellow F1 Hybrid) are prepared and sown as described below. Seeds are divided into five groups as shown in Table 4.

TABLE 4
UV-B treatment regimen for maize seeds

| Group 1 | Group 2 | Group 3 | Group 4 | Group 5 |
|---|---|---|---|---|
| No priming, | Priming + | Priming + | Priming + | Priming + |
| No UV | UV-B* | No UV-B | UV-B | No UV-B |
| | (13 kJ m⁻²) | (13 kJ m⁻²) | (100 kJ m⁻²) | (100 kJ m⁻²) |

*UV-B wavelength ~280 nm, treatment time is 9 h or 21 h as above.

Maize seeds are immersed in water and kept in the dark at 16° C. After 16 hours, seeds are irradiated with 500 μmol m⁻²s⁻¹ of continuous red/blue light. Fifty percent of these seeds are additionally treated with UV-B. The UV is set to have irradiance in a range between 4×10⁻⁵ to 1.3×10⁻⁴ wcms⁻¹ using UV-B light supplied by a UV-LED source, the transmittance of which peaks at ˜280 nm. After 9 hours or 21 hours of treatment, seeds are air-dried for 72 hours at 16° C. then sown. Seedlings are harvested at 4 weeks old, and fresh and dry weights of shoots and roots are quantified. Indices for leaf chlorophyll, flavonoid, anthocyanin, and nitrogen index are assessed using a Dualex Scientific+ chlorophyll and polyphenol meter (Fore-A, Orsay, France).

Plants in Groups 2 and 4 have enhanced growth and appear healthy when compared with plants in Groups 1, 3, and 5, where the plants are not treated with the specific UV-B wavelength and dose. Plants in Groups 2 and 4 have increased root dry weight, root dry weight, whole plant dry weight, chlorophyll, flavonoid, anthocyanin, and nitrogen index. Plants in Group 4 appear to have higher growth rate with increased root dry weight, root dry weight, whole plant dry weight by at least 10% when compared with Group 2 plants, which received a different dose of UV-B from the Group 4 plants. Plants in Group 4 also have a higher, e.g., at least 5% increase, leaf chlorophyll, flavonoid, anthocyanin, and nitrogen index when compared with plants in Group 2.

The results demonstrate that UV-B treated in seeds or seed embryos provides beneficial effect to plants in enhancing the plant growth. The results also demonstrate that a UV-B dose between 13-100 kJ m⁻² is sufficient to stimulate plant growth. The results show that a particular of UV-B dose, e.g., a dose at 100 kJ m⁻² for 21 hours, when exposed to plant seeds prior to sowing provides better subsequent plant performance. This effect; however, may vary when the duration of UV-B treatment is decreased or increased.

### Example 4: UV-B Treatment in Seeds/Seed Embryos Increases Plant Drought Resistance

This example illustrates the beneficial effects of UV-B treatment in seeds or seed embryos prior to sowing. Plants grown from seeds or seed embryos are handled and prepared for UV-B treatment as described in Example 1 exhibits enhanced growth or increased drought resistance when compared to their non-UB-B treatment counterparts.

Seeds of kale are prepared and sown as described below. Seeds are divided into five groups as shown in Table 5.

TABLE 5
UV-B treatment regimen for kale seeds

| Group 1 | Group 2 | Group 3 | Group 4 | Group 5 |
|---|---|---|---|---|
| No priming, | Priming + | Priming + | Priming + | Priming + |
| No UV | UV-B* | No UV-B | UV-B | No UV-B |
| | (13 kJ m⁻²) | (13 kJ m⁻²) | (100 kJ m⁻²) | (100 kJ m⁻²) |

*UV-B wavelength ~280 nm, treatment time is 1 minute.

Kale seeds are immersed in water and kept in the dark at 16° C. After 4 hours, seeds are irradiated with 500 μmol m⁻²s⁻¹ of continuous red/blue light. Fifty percent of these seeds are additionally treated with UV is set to have irradiance in a range between 4×10⁻⁵ to 1.3×10⁻⁴ wcms⁻¹ using UV-B light supplied by a UV-LED source, the transmittance of which peaks at ˜280 nm. After 30 hours of treatment, seeds are air-dried for 72 hours at 16° C. Seeds then subjected to a drought stress during germination. UV-treated and control seeds are germinated in well-watered medium, drought medium (concentrations of PEG8000 at −1 mPA) or severe drought medium (concentrations of PEG8000 at −1.5 mPA). After 72 hours, seedling weight and radicle length are quantified.

Plants in Groups 2 and 4 have enhanced resistance to drought and appear healthy when compared with plants in Groups 1, 3, and 5, where the plants are not treated with the specific UV-B wavelength and dose. Plants in Groups 2 and 4 have increased radicle length and biomass in well-water medium, drought medium and severe drought medium. Plants in Groups 2 and 4 appear to have better performance with increased radical length by at least 10% and increased biomass by at least 10% when compared with Groups 3 and 5 plants, which are not primed.

The results demonstrate that UV-B treated in seeds or seed embryos provides beneficial effect to plants in enhancing the plant growth. The results also demonstrate that a UV-B dose between 13-100 kJ m⁻² is sufficient to provide protection against yield-limiting stresses encountered in the growing environment, such as drought or salinity stress. This effect; however, may vary when the duration of UV-B treatment is decreased or increased.

### Example 5: UV-B Treatment in Plants Increases Plant Performance

This example illustrates the beneficial effects of UV-B treatment in immature plants. Plants treated with UV-B in early development exhibit enhanced growth or performance when compared to their non-UB-B treatment counterparts.

Young maize plants are handled and treated with UV-B as described below. Young maize plants are divided into three groups as shown in Table 6.

TABLE 6
UV-B treatment regimen for maize seeds

| Group 1 | Group 2 | Group 3 |
|---|---|---|
| No priming, | Priming + | Priming + |
| No UV | UV-B* (13 kJ m⁻²) | No UV-B (13 kJ m⁻²) |

*UV-B wavelength ~280 nm, treatment time is 9 h.

Young maize plants are irradiated with 500 μmol m⁻²s⁻¹ of continuous red/blue light. Fifty percent of these immature plants are additionally treated with 1.3×10⁻⁴ Wcm⁻²s⁻¹ UV-B light supplied by a UV-LED source, the transmittance of which peaks at ˜280 nm. After 9 hours of treatment, immature plants are divided into three groups and inspected for performance as described in Table 7.

TABLE 7
Performance of immature plants subjected to UV-B treatment

| | Group A | Group B | |
|---|---|---|---|
| | (resistance to | (resistance to | Group C |
| | biotic stress) | abiotic stress) | (growth performance) |
| Inspection | Leaf disease | Drought | Fruit |
| parameter | Ear rot disease | Salinity | Flower |
| | Stalk rot disease | | Height |
| | Seeding and root | | Leaf surface area |
| | disease | | Hormone index, e.g. |
| | | | flavonoid, anthocyanin |
| | | | Seed germination rate |
| | | | Nitrogen index |
| | | | Hardiness |

The immature plants are treated with UV-B and inspected for various performances as described in Examples 2, 3 and 4. The UV-B treated immature plants are allowed to grow to maturity and samples are collected, depending on the type of inspection.

For biotic stress test, mature plants are inspected for leaf disease, ear rot disease, stalk rot disease, and seeding and root disease.

Plants in Groups 2 have enhanced resistance to biotic stress, abiotic stress and growth performance when compared with plants in Groups 1 and 3. Plants in Group 2 have reduced rate of infections relate to leaf disease, ear rot disease, stalk rot disease, and/or seedling and root diseases. Group 2 plants also have increased resistance to drought or increased salinity as demonstrated by, e.g., increased biomass dry weight. Group 2 plants also have better growth performance including increased fruit size, improved fruit tastes, increased leaf surface area, increased flowering, increased height, increased hormone index, e.g. flavonoid, anthocyanin, increased nitrogen index, increased hardiness, and/or increased seed germination rate.

The results demonstrate that UV-B treated in immature plants provides beneficial effect to plants in enhancing the plant resistance to biotic stress, abiotic stress, and plant performance.

### Example 6: UV-B Exposure in Seeds or Seed Embryos Activates Gene in the UVR8-COP1-HY5 UV-B Signaling Pathway

This experiment illustrates the beneficial effects of UV-B treatment in seeds or seed embryos. Seeds or seed embryos that are treated with UV-B exhibits enhanced physiological conditions such as improved growth, and or enhanced immune defense when compared to their non-UV-B treatment counterparts. The genes responsible for these physiological conditions are isolated and identified.

Seeds or seed embryos are handled and treated with UV-B as described in Example 1-5. The treated seeds or seed embryos are sown and allowed to germinate and grow into seedlings and subsequently mature plants. Plant samples from UV-B treated (UV-B) and non-UV-B treated group (control) are collected at various time points: during seed germination, during seedling stage, in immature plant, in mature plant, in flowers and in fruits. The plant samples are subjected for RNA extraction.

To access upregulation of a gene, RNA is extracted from the plant samples collected from various time points from the UV-B and control plant groups. cDNA is synthesized from the collected RNA and the quantification of gene expression is measured by quantitative-PCR (Q-PCR) or microarray. The expression level of a gene in the UV-B group is compared to a corresponding gene found in the control group. Genes found to have upregulated quantification in the UV-B group are isolated and sequenced, e.g. using Illumina sequencing platform.

The identified genes and their functions are correlated by the time of plant sample collection and the plant physiological condition. The identified genes that are related to the UVR8-COP1-HY5 UV-B signaling pathway are verified or homologous sequences disclosed in the field. Some identified genes do not match with the available UVR8-COP-1-HY5 UV-B signaling pathway responsive gene list. The function of these genes in response to UV-B treatment has not been reported. Further analysis will be conducted to correlate the function of these novel genes and their effect on plant development.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the appended claims define the scope of the disclosure and that methods and structures within the scope of these claims and their equivalents be covered thereby.

1. A transgenic plant comprising an isolated polynucleotide comprising a nucleic acid sequence encoding a modulator that is responsive to UV-B administration in a plant material of the transgenic plant, wherein the transgenic plant produces at least one enhanced phenotype in an absence of supplementary UV-B irradiation, and wherein the at least one enhanced phenotype is selected from the group consisting of increased crop yield, growth rate, hardiness, stress resistance, and pathological resistance when compared to a plant lacking the modulator.

2. The transgenic plant of claim 1, wherein the modulator is a modulator of UVR8-COP1-HY5 UV-B signaling pathway.

3. The transgenic plant of claim 1, wherein the modulator is selected from a group of genes consisting of Hy5, CHS, COP1, UVR8, HYH, GPX7, SIG5, CRY3, ELIP1, SWA3, PHYA, FAR1, FHY3, FHY1FHL, MYB111, MYB12, MKP1, PAP1, C4H, MYB4, AtMYB12, AtCHS, and AtC4H.

4. The transgenic plant of claim 1, wherein the modulator when expressed activates a downstream regulator of UVR8-COP1-HY5 UV-B signaling pathway.

5. The transgenic plant of claim 1, wherein the modulator when expressed increases accumulation of a transcript encoding a member of UVR8-COP1-HY5 UV-B signaling pathway.

6. The transgenic plant of claim 1, wherein the modulator when expressed reduces a suppressor of UVR8-COP1-HY5 UV-B signaling pathway.

7. (canceled)

8. (canceled)

9. (canceled)

10. (canceled)

11. The transgenic plant of claim 1, further comprising a transgenic tissue-specific promoter.

12. The transgenic plant of claim 11, wherein the tissue-specific promoter comprises at least one of a fruit, ovule-, carpel-, embryo-, pericarp-, endosperm-, pollen-, root-, leaf-, stem-, and a flower-specific promoter.

13. The transgenic plant of claim 1, further comprising a polynucleotide for increasing a level of an endogenous plant hormone.

14. The transgenic plant of claim 13, wherein the plant hormone is selected from the group consisting of auxins, gibberellins, cytokinins, and brassinosteroids.

15. (canceled)

16. The transgenic plant of claim 13, wherein the plant hormone is selected from the group consisting of auxins, gibberellins, cytokinins, and brassinosteroids.

17. (canceled)

18. (canceled)

19. The transgenic plant of claim 1, further comprising a constitutive promoter.

20. The transgenic plant of claim 1, wherein the transgenic plant has improvement of a physiological condition characterized by an increase in at least one of dry weight, shoot fresh weight, pigment production, radical length, leaf size, and nitrogen index.

21. The transgenic plant of claim 1, wherein the phenotype is enhanced by at least 5%.

22. (canceled)

23. (canceled)

24. (canceled)

25. The transgenic plant of claim 1, wherein the transgenic plant is selected from the group consisting of lettuce, beans, broccoli, cabbage, carrot, cauliflower, cucumber, melon, onion, peas, peppers, pumpkin, spinach, kale, squash, sweetcorn, corn, maize, tomato, watermelon, alfalfa, canola, cotton, sorghum, soybeans, sugar beets, wheat, rice, grass, and flowering plants.

26. The transgenic plant of claim 1, wherein the transgenic plant is an indoor plant.

27. The transgenic plant of claim 1, wherein the transgenic plant is an outdoor plant.

28. The transgenic plant of claim 1, wherein the plant material comprises at least one of a seed, a seedling, and a plant.

29. (canceled)

30. The transgenic plant of claim 1, wherein the transgenic plant is grown from a transgenic seed.

31. A transgenic seed comprising an isolated polynucleotide comprising a nucleic acid sequence encoding a modulator that is responsive to UV-B administration, wherein the transgenic seed produces at least one enhanced phenotype in an absence of supplementary UV-B irradiation, and wherein the at least one enhanced phenotype is selected from the group consisting of increased crop yield, growth rate, hardiness, stress resistance, and pathological resistance when compared to a seed lacking the modulator.
